(19) United States
(12) Patent Application Publication
(10) Pub. No.: US 2023/0330129 A1
(43) Pub. Date: Oct. 19, 2023

(54) TUMOR NECROSIS FACTOR RECEPTOR SUPERFAMILY (TNFRSF) AGONISTS, SPHERICAL NUCLEIC ACID (SNA) TLR9 AGONISTS AND CHECKPOINT INHIBITORS FOR ANTITUMOR THERAPY

(71) Applicant: Exicure Operating Company, Chicago, IL (US)

(72) Inventors: Bart Anderson, Morton Grove, IL (US); Pinal Patel, Chicago, IL (US)

(73) Assignee: Exicure Operating Company, Chicago, IL (US)

(21) Appl. No.: 18/020,501

(22) PCT Filed: Aug. 11, 2021
(86) PCT No.: PCT/US2021/045620

§ 371 Date: Feb. 9, 2023

Related U.S. Application Data
(60) Provisional application No. 63/078,866, filed on Sep. 15, 2020.
(60) Provisional application No. 63/064,304, filed on Aug. 11, 2020.

(51) Int. Cl. A61K 31/7125; A61K 9/127; A61P 35/00; A61K 39/395

(52) U.S. Cl. CPC A61K 31/7125; A61K 9/127; A61P 35/00; A61K 39/3955; A61K 2039/505

(57) ABSTRACT

Aspects of the disclosure relate to the administration of at least one of spherical nucleic acids (SNAs), a checkpoint inhibitor antibody, and an agonist of a tumor necrosis factor receptor superfamily (TNFRSF) member at various doses, schedules of administration, orders of administration, etc. for treatment of a disease, such as cancer.

### RELATED APPLICATIONS

This application claims the benefit of the filing dates under 35 U.S.C. § 119(e) of U.S. Provisional Application No. 63/064,304, entitled “TUMOR NECROSIS FACTOR RECEPTOR SUPERFAMILY (TNFRSF) AND SPHERICAL NUCLEIC ACID (SNA) TLR9 AGONISTS”, filed on Aug. 11, 2020, and of U.S. Provisional Application No. 63/078,866, entitled “COMBINED SPHERICAL NUCLEIC ACID AND CHECKPOINT INHIBITOR FOR ANTITUMOR THERAPY”, filed on Sep. 15, 2020, the contents of each of which are incorporated herein by reference in their entireties.

### BACKGROUND

Spherical nucleic acid (SNA) constructs are three-dimensional arrangements of oligonucleotides that utilize scavenger receptors to enter cells; this contrasts with other oligonucleotide delivery systems, such as those utilizing cationic lipids or polymers, which often disrupt anionic cell membranes to deliver payloads. Immune checkpoints are inhibitory pathways in the immune system that are crucial for both maintaining self-tolerance and for modulating the duration and amplitude of physiological immune responses. These pathways are critical in minimizing collateral tissue damage by the immune system. Lastly, the tumor necrosis factor receptor superfamily (TNFRSF) is a protein superfamily of cytokine receptors characterized by the ability to bind tumor necrosis factors (TNFs) via an extracellular cysteine-rich domain.

### SUMMARY

According to some aspects, disclosed herein are methods for treating a disease. In some embodiments, the method for treating a disease comprises administration of an agonist of a tumor necrosis factor receptor superfamily (TNFRSF) member and a spherical nucleic acid (SNA) to a subject to treat a disease in the subject, wherein the SNA comprises a core and an oligonucleotide shell comprised of CpG oligonucleotides positioned on the exterior of the core, and wherein the administration of the SNA comprises administration once a week, once every three weeks, or once a week followed by once every three weeks.

In some embodiments, the at least one CpG oligonucleotide is linked to the exterior surface of the core through a spacer or each CpG oligonucleotide is linked to the exterior surface of the core through a spacer, and the core has a diameter of less than 40 nm.

In some embodiments, the core is a liposome core.

In some embodiments, the disease is cancer.

In some embodiments, administration of the SNA comprises administration once a week for one week to 12 weeks, followed by once every three weeks. In some embodiments, administration of the SNA comprises administration once a week for nine weeks, followed by once every three weeks.

In some embodiments, administration of the agonist of a TNFRSF member comprises administration once a week, once every two weeks, once every three weeks, once every four weeks, or a combination thereof. In some embodiments, administration of the agonist of a TNFRSF member comprises administration once every three weeks. In some embodiments, administration of the agonist of a TNFRSF member comprises administration once a week.

In some embodiments, administration of the SNA is via at least one route of administration chosen from intratumoral administration to a solid tumor or tumor lesion, subcutaneous administration, internal implantation and intravenous administration.

In some embodiments, administration of the agonist of a TNFRSF member is via at least one route of administration chosen from intratumoral administration to a solid tumor or tumor lesion, subcutaneous administration and intravenous administration.

In some embodiments, administration of the SNA is via at least one route of administration chosen from intratumoral administration to a solid tumor or tumor lesion, subcutaneous administration, internal implantation and intravenous administration, and administration of the agonist of a TNFRSF member is via intravenous administration.

In some embodiments, administration of the SNA is via at least one route of administration chosen from intratumoral administration to a solid tumor or tumor lesion, subcutaneous administration, internal implantation and intravenous administration, and administration of the agonist of a TNFRSF member is via at least one route of administration chosen from intratumoral administration to a solid tumor or tumor lesion and subcutaneous administration.

In some embodiments, the method further comprises administering an immune checkpoint inhibitor to the subject.

In some embodiments, administration of the SNA is within 24 hours of administration of the agonist of a TNFRSF member. In some embodiments, administration of the SNA is within 24 hours of administration of the immune checkpoint inhibitor.

In some embodiments, administration of the SNA is within 24 hours of administration of the immune checkpoint inhibitor and within 24 hours of administration of the agonist of a TNFRSF member.

In some embodiments, the cancer in the subject is not responsive to treatment with an immune checkpoint inhibitor alone or the cancer in the subject is resistant to treatment with an immune checkpoint inhibitor alone.

In some embodiments, administration of the SNA is at a dose of or about between 1 mg and 50 mg. In some embodiments, administration of the SNA is at a dose of or about between 2 mg and 32 mg. In some embodiments, administration of the immune checkpoint inhibitor is at a dose of or about between 150 mg and 850 mg. In some embodiments, administration of the agonist of a TNFRSF member is at a dose of or about between 7 mg and 1400 mg.

In some embodiments, the cancer is skin cancer, biliary tract cancer, brain cancer, breast cancer, cervical cancer, choriocarcinoma, colon cancer, endometrial cancer, esophageal cancer, gastric cancer, an intraepithelial neoplasm, leukemia, lymphoma, liver cancer, lung cancer, oral cancer, ovarian cancer, pancreatic cancer, prostate cancer, rectal cancer, testicular cancer, thyroid cancer, renal cancer, multiple myeloma, or bladder cancer. In some embodiments, the cancer is cutaneous squamous cell carcinoma, Merkel cell carcinoma, basal cell carcinoma, melanoma, hepatocellular carcinoma, neuroblastoma, glioma, glioblastoma multiforme, pancreatic adenocarcinoma, hormone refractory prostate adenocarcinoma, anaplastic thyroid cancer, hairy cell leukemia, chronic myelogenous leukemia, cutaneous T-cell leukemia, renal cell carcinoma, clear cell renal cell carcinoma, or non-small cell lung cancer (NSCLC).

In some embodiments, the cancer is cutaneous squamous cell carcinoma, Merkel cell carcinoma, melanoma, or basal cell carcinoma (BCC). In some embodiments, the cancer is liver cancer, optionally wherein the liver cancer is hepatocellular carcinoma (HCC).

In some embodiments, the cancer is not breast cancer.

In some embodiments, the cancer is a sarcoma.

In some embodiments, the sarcoma is pleomorphic sarcoma, gastrointestinal stromal tumor (GIST), liposarcoma, leiomyosarcoma, synovial sarcoma, malignant peripheral nerve sheath tumor, rhabdomyosarcoma, angiosarcoma, fibrosarcoma, dermatofibrosarcoma protuberans, epithelioid sarcoma, myxoma, mesenchymoma, vascular sarcoma, neurilemmoma, bone sarcoma, osteosarcoma, Ewing's sarcoma, chondrosarcoma, Kaposi sarcoma, solitary fibrous tumor, chordoma, desmoid-type fibromatosis, fibroblastic sarcoma, giant cell tumor of the bone, gynecological sarcoma, soft tissue sarcoma, angioleiomyoma, leiomyoma, smooth muscle sarcoma, or fibrohistiocytic sarcoma.

In some embodiments, the subject has a solid tumor or a tumor lesion that can be injected intratumorally via one or more of palpation and ultrasound.

In some embodiments, the solid tumor or tumor lesion is on or near the skin, on or near cutaneous soft tissue, on or near subcutaneous soft tissue, and/or in or near a lymph node.

In some embodiments, administration of the SNA is to at least one lesion chosen from a cutaneous tumor lesion, a subcutaneous tumor lesion and a nodal lesion.

In some embodiments, the spacer is or comprises an oligoethylene glycol. In some embodiments, oligoethylene glycol is a hexaethylene glycol.

In some embodiments, the SNA includes between about 25 and about 35 CpG oligonucleotides.

In some embodiments, at least one CpG oligonucleotide comprises or consists of the nucleic acid sequence of CpG-7909 or each CpG oligonucleotide comprises or consists of the nucleic acid sequence of CpG-7909. In some embodiments, at least one CpG oligonucleotide comprises or consists of the nucleic acid sequence 5′-TCGTCGTTTTGTCGTTTTGTCGTT-3′ (SEQ ID NO: 1) or each CpG oligonucleotide comprises or consists of the nucleic acid sequence 5′-TCGTCGTTTTGTCGTTTTGTCGTT-3′ (SEQ ID NO: 1).

In some embodiments, at least one CpG oligonucleotide comprises or consists of the sequence 5′-T*C*G*T*C*G*T*T*T*T*G*T*C*G*T*T*T*T*G*T*C*G*T*T-3′/HEG/HEG/TEG Cholesteryl Ester/ (SEQ ID NO: 1) or each CpG oligonucleotide comprises or consists of the sequence 5′-T*C*G*T*C*G*T*T*T*T*G*T*C*G*T*T*T*T*G*T*C*G*T*T-3′/HEG/HEG/TEG Cholesteryl Ester/ (SEQ ID NO: 1).

In some embodiments, the immune checkpoint inhibitor is an anti-PD-1 antibody, an anti-PD-L1 antibody, or an anti-CTLA4 antibody. In some embodiments, the immune checkpoint inhibitor is an anti-PD-1 antibody. In some embodiments, the anti-PD-1 antibody is retifanlimab.

In some embodiments, the TNFRSF member is OX40. In some embodiments, the agonist of TNFRSF member is an agonist of OX40. In some embodiments, the agonist of OX40 is an anti-OX40 antibody. In some embodiments, the anti-OX40 antibody is INCAGN1949. In some embodiments, the anti-OX40 antibody is PF-04518600. In some embodiments, the anti-OX40 antibody is GSK3174998. In some embodiments, the anti-OX40 antibody is BMS-986178. In some embodiments, the anti-OX40 antibody is tavolixizumab (MEDI 0562). In some embodiments, the anti-OX40 antibody is pogalizumab (MOXR0916/RG7888). In some embodiments, the anti-OX40 antibody is ABBV-368.

In some embodiments, the TNFRSF member is 4-1BB (CD137). In some embodiments, the agonist of TNFRSF member is an agonist of 4-1BB. In some embodiments, the agonist of 4-1BB is an anti-4-1BB antibody. In some embodiments, the anti-4-1BB antibody is utomilumab (PF-05082566).

In some embodiments, administration of the SNA is within 12 hours of administration of the agonist of a TNFRSF member. In some embodiments, administration of the SNA is within 12 hours of administration of the immune checkpoint inhibitor. In some embodiments, administration of the SNA is within 12 hours of administration of the immune checkpoint inhibitor and within 12 hours of administration of the agonist of a TNFRSF member.

In some embodiments, the core has a diameter that is less than about 30 nm. In some embodiments, the core has a diameter that is between about 15 nm and about 40 nm. In some embodiments, the core has a diameter that is between about 15 nm and about 30 nm.

In some embodiments, administration of the SNA and the immune checkpoint inhibitor is substantially at the same time. In some embodiments, administration of the SNA is prior to administration of the agonist of a TNFRSF member.

In some embodiments, administration of the SNA is prior to administration of the immune checkpoint inhibitor or prior to administration of the immune checkpoint inhibitor and after administration of the agonist of a TNFRSF member.

In some embodiments, administration of the SNA is after administration of the agonist of a TNFRSF member. In some embodiments, administration of the SNA is after administration of the immune checkpoint inhibitor or after administration of the immune checkpoint inhibitor and after administration of the agonist of a TNFRSF member.

In some embodiments, administration of the SNA and the agonist of a TNFRSF member, or administration of the SNA, the agonist of a TNFRSF member and the immune checkpoint inhibitor results in one or more of increased cytokine expression, increased chemokine expression, or increased immune cell activation by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99%, at least or about 100%, or at least or about 150%.

In some embodiments, administration of the SNA and the agonist of a TNFRSF member, or administration of the SNA, the agonist of a TNFRSF member and the immune checkpoint inhibitor results in one or more of increased cytokine expression, increased chemokine expression, or increased immune cell activation by at least or about 2-fold, at least or about 3-fold, at least or about 4-fold, at least or about 5-fold, at least or about 6-fold, at least or about 7-fold, at least or about 8-fold, at least or about 9-fold, at least or about 10-fold, at least or about 15-fold, at least or about 20-fold, at least or about 30-fold, at least or about 40-fold, at least or about 50-fold or more, relative to a reference level.

In some embodiments, administration of the SNA and the agonist of a TNFRSF member, or administration of the SNA, the agonist of a TNFRSF member and the immune checkpoint inhibitor increases an immune cell population in a solid tumor or tumor lesion in the subject.

In some embodiments, the cancer in the subject is progressive disease (PD) and administration of the SNA and the agonist of a TNFRSF member, or the administration of the SNA, the immune checkpoint inhibitor and the agonist of a TNFRSF member renders the cancer stable disease.

In some embodiments, each CpG oligonucleotide comprises at least one phosphorothioate internucleotide linkage. In some embodiments, each internucleotide linkage of each CpG oligonucleotide is a phosphorothioate internucleotide linkage.

In some embodiments, the CpG oligonucleotides do not comprise a nucleotide in L-conformation. In some embodiments, the CpG oligonucleotides consist of nucleotides in D-conformation.

According to some aspects, disclosed herein are methods for treating cancer. In some embodiments, a method for treating cancer comprises:
administering to a subject a spherical nucleic acid (SNA) and a checkpoint inhibitor to treat the cancer in the subject,
wherein the SNA comprises a core having an oligonucleotide shell comprised of CpG oligonucleotides positioned on the exterior of the core, wherein the SNA is administered to the subject at a dose of at least about 2 mg to one solid tumor or tumor lesion or divided among two or more solid tumors or tumor lesions in the subject,
wherein the SNA is administered within 24 hours of administration of the checkpoint inhibitor.

In some embodiments, a method for treating cancer comprises:
administering to a subject a spherical nucleic acid (SNA) and a checkpoint inhibitor to treat cancer in the subject,
wherein the SNA comprises a core having an oligonucleotide shell comprised of CpG oligonucleotides positioned on the exterior of the core, wherein the SNA is administered at a dose of between 2 mg and 32 mg once a week or every three weeks,
wherein the checkpoint inhibitor is administered at a dose of between 180 and 370 mg every three weeks, or at a dose of between 700 mg and 900 mg every two weeks,
wherein the SNA is administered within 24 hours of the administration of the checkpoint inhibitor, and
wherein the SNA and the checkpoint inhibitor are administered through different routes of administration, wherein the cancer is basal cell carcinoma (BCC).

In some embodiments, a method for treating cancer comprises:
administering a therapeutic dose of a checkpoint inhibitor and a therapeutic dose of a spherical nucleic acid (SNA) to treat cancer in the subject, wherein the SNA comprises a CpG oligonucleotide linked through a spacer to an exterior surface of a liposome core having a diameter of less than 40 nm, wherein the SNA is administered by intratumoral (IT) administration into one tumor lesion or into multiple lesions at a dose of between 2 mg and 32 mg and wherein the checkpoint inhibitor is administered by intravenous (IV) administration at a dose of between 180 and 800 mg.

In some embodiments, the cancer is basal cell carcinoma (BCC). In some embodiments, the cancer is melanoma. In some embodiments, the cancer is liver cancer. In some embodiments, the liver cancer is hepatocellular carcinoma (HCC).

In some embodiments, the SNA is administered by subcutaneous administration or intratumoral administration to a solid tumor and the checkpoint inhibitor is administered by IV infusion.

In some embodiments, the cancer in the subject is not responsive to treatment with the checkpoint inhibitor alone or the cancer in the subject is resistant to treatment with the checkpoint inhibitor alone.

In some embodiments, the subject has not received a small molecule or tyrosine kinase inhibitor within 2 weeks or 5 half-lives (whichever is longer) prior to the first dose of the SNA, has not received chemotherapy within 3 weeks prior to the first dose of the SNA, has not received biological cancer therapy within 3 weeks prior to the first dose of the SNA, has not received nitrosourea or radioisotope within 6 weeks prior to first dose of the SNA, has not recovered from an adverse event (G1) or has not been identified as experiencing an adverse event due to cancer therapeutics administered more than 4 weeks prior to the first dose of the SNA.

In some embodiments, the SNA is administered at a dose of or about 16 mg. In some embodiments, the SNA is administered at a dose of or about 32 mg.

In some embodiments, the checkpoint inhibitor is administered at a dose between 100 mg and 1000 mg. In some embodiments, the checkpoint inhibitor is administered at a dose of or about 200 mg, 350 mg, or 800 mg.

In some embodiments, the subject has a solid tumor or a tumor lesion that can be injected intratumorally via one or more of palpation or ultrasound. In some embodiments, the solid tumor or tumor lesion is on or near the skin, on or near cutaneous soft tissue, on or near subcutaneous soft tissue, and/or in or near a lymph node.

In some embodiments, the SNA is administered to one or more of a cutaneous tumor lesion, a subcutaneous tumor lesion or a nodal lesion.

In some embodiments, the core is a liposome core. In some embodiments, the liposome core is about 15 nanometers (nm) to about 30 nm in diameter.

In some embodiments, each CpG oligonucleotide comprises a spacer. In some embodiments, the spacer consists of or comprises an oligoethylene glycol. In some embodiments, the oligoethylene glycol is a hexaethylene glycol.

In some embodiments, the SNA has 25 to 35 CpG oligonucleotides positioned on the exterior surface of the core.

In some embodiments, the CpG oligonucleotides comprise or consist of the nucleotide sequence 5′-TCGTCGTTTTGTCGTTTTGTCGTT-3′ (SEQ ID NO: 1). In some embodiments, the CpG oligonucleotides comprise or consist of the sequence 5′-T*C*G*T*C*G*T*T*T*T*G*T*C*G*T*T*T*T*G*T*C*G*T*T-3′/HEG/HEG/TEG Cholesteryl Ester/ (SEQ ID NO: 1).

In some embodiments, the checkpoint inhibitor is a programmed death (PD) 1 antibody or a programmed death-ligand (PD-L) 1 antibody. In some embodiments, the checkpoint inhibitor is pembrolizumab, avelumab, or cemiplimab.

In some embodiments, the SNA is administered within 24 hours of administration of the checkpoint inhibitor. In some embodiments, the SNA is administered within 12 hours of administration of the checkpoint inhibitor.

In some embodiments, the liposome core is less than 30 nm in diameter. In some embodiments, the liposome core is about 15 nm to 40 nm in diameter.

In some embodiments, the SNA and the checkpoint inhibitor are administered substantially at the same time.

In some embodiments, the SNA is administered prior to administration of the checkpoint inhibitor. In some embodiments, the SNA is administered after the administration of the checkpoint inhibitor.

In some embodiments, a method for treating cancer comprises:
administering to a subject a spherical nucleic acid (SNA) and a checkpoint inhibitor to treat the cancer in the subject,
wherein the SNA comprises a core and an oligonucleotide shell comprised of CpG oligonucleotides positioned on the exterior of the core, wherein the SNA is administered to the subject at a dose of between about 16 mg to about 32 mg to one solid tumor or tumor lesion or divided among two or more solid tumors or tumor lesions, wherein the SNA is administered within 24 hours of administration of the checkpoint inhibitor, and wherein the cancer is basal cell carcinoma (BCC), melanoma, or liver cancer.

In some embodiments, a method for treating cancer comprises:
administering to a subject a spherical nucleic acid (SNA) and a checkpoint inhibitor to treat the cancer in the subject,
wherein the SNA comprises a core and an oligonucleotide shell comprised of CpG oligonucleotides positioned on the exterior of the core, wherein the SNA is administered at a dose of between about 16 mg to about 32 mg once a week or once every three weeks,
wherein the checkpoint inhibitor is administered at a dose of between 180 mg and 370 mg every three weeks or at a dose of between 700 mg and 900 mg every two weeks,
wherein the SNA is administered within 24 hours of the administration of the checkpoint inhibitor,
wherein the SNA and the checkpoint inhibitor are administered through different routes of administration, wherein the cancer is basal cell carcinoma (BCC), melanoma, or liver cancer.

In some embodiments, a method for treating cancer comprises:
administering a therapeutic dose of a spherical nucleic acid (SNA) comprising a CpG oligonucleotide linked through a spacer to an exterior surface of a liposome core having a diameter of less than about 40 nanometers (nm) and a checkpoint inhibitor, wherein the SNA is administered by intratumoral (IT) administration into two or more solid tumors or tumor lesions at a dose of between about 16 mg and about 32 mg and the checkpoint inhibitor is administered by intravenous injection at a dose of between 180 and 370 mg or between 700 mg and 900 mg, or between 800 mg and 1700 mg, and wherein the cancer is basal cell carcinoma (BCC), melanoma, or liver cancer.

In some embodiments, the cancer is BCC. In some embodiments, the cancer is melanoma. In some embodiments, the cancer is liver cancer, optionally wherein the liver cancer is hepatocellular carcinoma (HCC).

In some embodiments, the checkpoint inhibitor is a PD-1 inhibitor or a PD-L1 inhibitor. In some embodiments, the PD-L1 inhibitor is atezolizumab (Tecentriq).

In some embodiments, the PD-L1 inhibitor is administered at a dose of or about 1680 mg, 1200 mg, or 840 mg.

In some embodiments, the method further comprises administration of an antiangiogenesis agent. In some embodiments, the antiangiogenesis agent is a vascular endothelial growth factor (VEGF) antibody. In some embodiments, the VEGF antibody is a monoclonal VEGF antibody. In some embodiments, the VEGF monoclonal antibody is bevacizumab (Avastin). In some embodiments, the VEGF antibody is administered by IV administration (e.g., IV injection or IV infusion). In some embodiments, the VEGF antibody is administered once every three weeks. In some embodiments, the VEGF antibody is administered at a dose of or about 15 mg/kg.

In some embodiments, the PD-1 inhibitor is pembrolizumab (Keytruda). In some embodiments, the PD-1 inhibitor is nivolumab (Opdivo).

In some embodiments, the PD-1 inhibitor is administered at a dose of 1 mg/kg, 240 mg, or 480 mg.

In some embodiments, the method further comprises administration of a CTLA-4 inhibitor. In some embodiments, the CTLA-4 inhibitor is ipilimumab (Yervoy). In some embodiments, the CTLA-4 inhibitor is administered at a dose of 3 mg/kg or 10 mg/kg.

In some embodiments, the checkpoint inhibitor is administered via intravenous infusion. In some embodiments, the checkpoint inhibitor is administered once every two weeks, once every three weeks, once every four weeks or once every six weeks. In some embodiments, ipilimumab is administered once every three weeks, four times or at least four times.

In some embodiments, the SNA is administered at a dose of or about 16 mg. In some embodiments, the SNA is administered at a dose of or about 32 mg. In some embodiments, the SNA is administered at a dose of or about 16 mg and the checkpoint inhibitor is administered at a dose of or about 200 mg. In some embodiments, the SNA is administered at a dose of or about 32 mg and the checkpoint inhibitor is administered at a dose of or about 200 mg. In some embodiments, the SNA is administered at a dose of or about 16 mg and the checkpoint inhibitor is administered at a dose of or about 350 mg. In some embodiments, the SNA is administered at a dose of or about 32 mg and the checkpoint inhibitor is administered at a dose of or about 350 mg. In some embodiments, the SNA is administered at a dose of or about 16 mg and wherein the checkpoint inhibitor is administered at a dose of or about 800 mg. In some embodiments, the SNA is administered at a dose of or about 32 mg and wherein the checkpoint inhibitor is administered at a dose of or about 800 mg.

In some embodiments, administration of the SNA or the SNA in combination with the checkpoint inhibitor results in one or more of increased cytokine expression, increased chemokine expression, or increased immune cell activation by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99%, at least or about 100%, at least or about 150%, at least or about 2-fold, at least or about 3-fold, at least or about 4-fold, at least or about 5-fold, at least or about 6-fold, at least or about 7-fold, at least or about 8-fold, at least or about 9-fold, at least or about 10-fold, at least or about 15-fold, at least or about 20-fold, at least or about 30-fold, at least or about 40-fold, at least or about 50-fold or more, relative to a reference level.

In some embodiments, the cancer in the subject is progressive disease and administration of the SNA or administration of the SNA in combination with the checkpoint inhibitor for the treatment of the cancer in the subject renders the cancer stable disease. In some embodiments, the cancer is stable disease for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks. In some embodiments, the cancer is stable disease for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months. In some embodiments, the cancer is stable disease for at least one year, at least two years, at least three years, at least four years, at least five years, at least six years, at least seven years, at least eight years, at least nine years, at least 10 years, at least 11 years or at least 12 years.

In some embodiments, the subject has at least one target lesion, at least two target lesions, at least three target lesions or at least four target lesions and administration of the SNA or the SNA in combination with the checkpoint inhibitor decreases the diameter of at least one target lesion in the subject or decreases the sum of the diameters of two or more target lesions in the subject by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99% relative to a reference level.

In some embodiments, the subject has at least one target lesion, at least two target lesions, at least three target lesions or at least four target lesions and administration of the SNA or the SNA in combination with the checkpoint inhibitor results in partial response or results in complete response in at least one target lesion, at least two target lesions, at least three target lesions, or at least four target lesions in the subject, wherein the target lesion is a solid tumor or tumor lesion.

In some embodiments, the treatment results in partial response or complete response in the subject for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks. In some embodiments, the treatment results in partial response or complete response in the subject for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months. In some embodiments, the treatment results in partial response or complete response in the subject for at least one year, at least two years, at least three years, at least four years, at least five years, at least six years, at least seven years, at least eight years, at least nine years, at least 10 years, at least 11 years or at least 12 years.

Each of the limitations of the invention can encompass various embodiments of the invention. It is, therefore, anticipated that each of the limitations of the invention involving any one element or combinations of elements can be included in each aspect of the invention.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. For purposes of clarity, not every component may be labeled in every drawing. In the drawings:

FIG. 1 shows an overview of Study Design.

FIG. 2 shows monotherapy dose escalation rules.

FIG. 3 shows combination therapy dose escalation rules.

FIG. 4 shows individual patient decision making rules.

FIG. 5 shows total dose volume algorithm.

FIG. 6 shows SNA of CpG oligonucleotides tumor administration technique. Cycle 1 (SNA of CpG oligonucleotides monotherapy) will be 2 weeks long and comprise intratumoral (IT) injections on days 1 and 8. For Cycles 2 and 3, SNA of CpG oligonucleotides will be administered on a weekly basis, on days 1, 8, and 15 of each 3-week cycle. Thereafter (Cycle 4+), SNA of CpG oligonucleotides will be administered every 3 weeks on day 1 until lack of clinical benefit or disease progression.

FIG. 7 shows an overview of Study Design with Phase 2 expansion in Merkel cell carcinoma (MCC) and in cutaneous squamous-cell carcinoma (CSCC)

FIG. 8 shows dosing and assessment schedule.

FIGS. 9A-9D show combined tumor volume of right and left flank tumors in individual mice bearing S180 tumors, treated with PBS (FIG. 9A), anti-PD-1 (FIG. 9B), anti-OX40 (FIG. 9C), or anti-OX40 and anti-PD-1 (FIG. 9D).

FIGS. 10A-10B show the group-wise mean S180 tumor volume (mm³) of 5 animals per group (FIG. 10A) and mean group-wise percent change from day 0 of the individual animal S180 tumor volume of the 5 animals per group (FIG. 10B), for animals treated with PBS (G1-PBS), anti-PD-1 (G2-anti-PD1), anti-OX40 (G3-anti-OX40), or anti-PD-1 and anti-OX40 (G4-anti-PD1+anti-OX40). These data were derived from the data shown in FIGS. 9A-9D.

FIGS. 11A-11D show combined tumor volume of right and left flank tumors in individual mice bearing EMT6 tumors, treated with PBS (FIG. 11A), anti-PD-1 (FIG. 11B), anti-OX40 (FIG. 11C), or anti-OX40 and anti-PD-1 (FIG. 11D).

FIGS. 12A-12B show the group-wise mean EMT6 tumor volume (mm³) of 5 animals per group (FIG. 12A) and mean group-wise percent change from day 0 of the individual animal EMT6 tumor volume of the 5 animals per group (FIG. 12B), for animals treated with PBS (G1-PBS), anti-PD-1 (G2-anti-PD1), anti-OX40 (G3-anti-OX40), or anti-PD-1 and anti-OX40 (G4-anti-PD1+anti-OX40). These data were derived from the data shown in FIGS. 11A-11D.

FIG. 13 shows a sample dosing and assessment schedule for a combination treatment. CpG-7909-SNA can be administered intratumorally and anti-PD-1 antibody can be administered intravenously for the treatment of a subject with cancer, such as basal cell carcinoma (BCC), melanoma, and liver cancer (e.g., hepatocellular carcinoma or HCC).

### DETAILED DESCRIPTION

Antibodies targeting cytotoxic T-lymphocyte-associated protein 4 (CTLA-4), programmed death 1 (PD-1), and programmed death ligand 1 (PD-L1) are used for treatment of advanced melanoma, head and neck squamous cell carcinoma, classic Hodgkin's lymphoma, renal cell carcinoma, urothelial carcinoma, cutaneous squamous cell carcinoma, Merkel cell carcinoma, and non-small cell lung cancer. However, despite the promise of these therapies, there is room to improve response rates to these drugs. For example, the PD-1 inhibitor nivolumab produced response rates of up to 40% in advanced melanoma trials [Opdivo® USPI]. The use of a spherical nucleic acid (SNA) disclosed herein, such as CpG-SNA, which is comprised of CpG oligonucleotides that are agonists of the toll-like receptor (TLR) 9, increases the amplitude of the immune response against tumors while simultaneously inhibiting the immune checkpoints with an inhibitor antibody (e.g., an immune checkpoint inhibitor or immune checkpoint inhibitor antibody). The activation of the TLR9 by an SNA disclosed herein produces immune responses that are useful in oncology applications. TLR9 stimulation initially activates antigen-nonspecific innate immunity followed by antigen-specific adaptive immunity.

The activation of TLR9 can be used to treat or to enhance treatment (e.g., with anti-PD-1 antibody) of cancers, such as non-melanoma skin cancers, including basal cell carcinoma (BCC). BCC is the most common form of skin cancer and the cancer with the highest incidence, with an estimated more than four million cases diagnosed in the U.S. each year. BCC arises from malignant growth of basal cells in the epidermis of the skin and lesions commonly arise in sun-exposed areas of the skin. BCC lesions commonly have high tumor mutational burden (TMB) and are highly immunogenic. While superficial BCC can be treated with surgical excision, Mohs micrographic surgery, curettage and electrodessication, radiotherapy, cryotherapy, photodynamic therapy, and/or with topical treatment using the TLR7 agonist imiquimod or 5-fluorouracil, treatment of advanced BCC can be challenging and outcomes are poorer. Immune checkpoint inhibitor treatment (e.g., using anti-PD-1 antibodies) has been tested in locally advanced or metastatic BCC cases, but 70-80% of these cases demonstrate primary or secondary resistance to such treatment.

In some embodiments, one or more of the tumor lesions in BCC are located on or near the surface of the skin. In some embodiments, the BCC is nodular BCC. In some embodiments, the BCC is superficial BCC. In some embodiments, the BCC is morphoeic BCC. In some embodiments, the BCC is basosquamous carcinoma.

Superficial BCC grows slowly, has minimal tendency to be invasive, and appears clinically as an erythematous, well-circumscribed patch or plaque, often with a whitish scale. Occasionally, minute eschars may appear within the patch or plaque. The tumor often appears multicentric, with areas of clinically normal skin intervening among clinically involved areas.

Advanced BCC, including locally advanced and metastatic BCC, is often not amenable to surgery or radiation therapy. As such, advanced cases represent a major treatment challenge.

In some embodiments, the term “locally advanced BCC” may refer to large, deep, invasive, aggressive, or recurrent tumors, and may be broadly categorized as BCC for which initial or subsequent surgery are considered inappropriate and for which radiation therapy is considered inappropriate as a single modality or second-line treatment.

Metastatic BCC is BCC which has spread from the site of the primary tumor to one or more secondary sites within the body. These secondary sites may include lymph nodes, lungs, bones, distant sites in the skin, or other organs. Hedgehog inhibitors, vismodegib and sonidegib are approved for systemic treatment of certain subsets of patients with advanced BCC.

Co-stimulation or the provision of two distinct signals to T-cells is an established model of lymphocyte activation of resting T lymphocytes by antigen-presenting cells (APCs). This model also provides for the discrimination of “self” from “non-self” and immune tolerance. The primary signal, or antigen specific signal, is transduced through the T-cell receptor (TCR) following recognition of foreign antigen peptide presented in the context of the major histocompatibility-complex (MHC). The second or co-stimulatory signal is delivered to T-cells by co-stimulatory molecules expressed on APCs, and induce T-cells to promote clonal expansion, cytokine secretion and effector function. In the absence of co-stimulation, T-cells can become refractory to antigen stimulation, fail to mount an effective immune response, and may result in exhaustion or acquire tolerance to foreign antigens. Co-stimulation can occur through agonism of surface receptors on immune cells, and as such activation through pharmaceutical products including antibodies, aptamers, peptides, and proteins, especially in combination with an SNA (e.g., SNA of CpG oligonucleotides disclosed herein), enhances the activation of an immune response.

Administration of an SNA of CpG oligonucleotides disclosed herein and an agonist of a co-stimulatory molecule, such as an agonist of a tumor necrosis factor receptor superfamily (TNFRSF) member (e.g., TNFRSF4, OX40), stimulates an immune response, such as an adaptive immune response. In some embodiments, the stimulation of an immune response is useful in disease (e.g., cancer) treatment. In some embodiments, an SNA of CpG oligonucleotides disclosed herein, an agonist of a co-stimulatory molecule, such as an agonist of a tumor necrosis factor receptor superfamily (TNFRSF) member (e.g., TNFRSF4, OX40), and an immune checkpoint inhibitor are useful in disease (e.g., cancer) treatment.

CpG-7909-SNA is a SNA configuration of a TLR9 agonist oligonucleotide designed to trigger innate and adaptive immune responses in patients suffering from disease, such as cancer (e.g., BCC, melanoma, hepatocellular carcinoma (HCC)). As disclosed herein, CpG-7909-SNA can be administered through various routes of administration for the treatment of tumors, such as tumors in BCC, including but not limited to tumor lesions of locally advanced BCC and/or metastatic BCC, melanoma and liver cancer (e.g., HCC). CpG-7909-SNA can also be administered for the treatment of superficial BCC (e.g., small, early, and/or non-advanced tumor lesions) or BCC lesions located on or near the surface of the skin. In some embodiments, CpG-7909-SNA can be administered to one or more superficial BCC lesions in a patient with metastatic BCC (e.g., a patient having one or more metastatic lesions). In some embodiments, CpG-7909-SNA can be administered to one or more metastatic BCC lesions. In some embodiments, CpG-7909-SNA can be administered to one or more solid tumors or one or more solid lesions in melanoma. In some embodiments, CpG-7909-SNA can be administered to one or more solid tumors or one or more solid lesions in HCC. In some embodiments, administration of an SNA, such as an SNA of CpG-7909, is via intratumoral (IT) administration, subcutaneous (SC) administration, both IT administration and SC administration, or internal implantation for the treatment of solid tumors.

SNAs (e.g., SNA of CpG oligonucleotides) disclosed herein, exhibit a suite of mechanistic and functional properties that make it ideal for agonizing TLR9. First, SNAs are more efficiently taken into and concentrated in endosomes compared to linear oligonucleotides (i.e., oligonucleotides not in the SNA configuration). Second, the oligonucleotides delivered as a part of SNAs cause an enhanced cytokine response, both in magnitude and duration, compared to linear oligonucleotides. Third, the SNA projects its oligonucleotides outward, allowing it to act upon TLRs. This arrangement of nucleic acids or oligonucleotides in the SNA contrasts with other oligonucleotide delivery systems where the oligonucleotides are held, for instance, inside virus-like particles.

In some embodiments, an SNA comprises a densely packed oligonucleotide shell with oligonucleotides, such as CpG oligonucleotides, which are radially oriented and stimulate a toll-like receptor (TLR), such as TLR9, resulting in an immune response. In some embodiments, the oligonucleotides in the oligonucleotide shell are oriented radially around a core (e.g., a liposome core).

An SNA is a three-dimensional arrangement of nucleic acids or oligonucleotides (e.g., CpG oligonucleotides), comprising an oligonucleotide shell, with densely packed and radially arranged oligonucleotides on the exterior of a core. The SNA is composed of oligonucleotides and a core. The core may be a hollow core which is produced by a three-dimensional arrangement of molecules which form the exterior surface of the core. For instance, the molecules may be in the form of a lipid layer (e.g. lipid monolayer or lipid bilayer), which has a hollow center (e.g., a non-lipid center, a non-solid center, etc.). In some embodiments, the core may be a liposome or liposome core. In some embodiments, the core is a liposome core.

In some embodiments, molecules may be in the form of lipids, such as amphipathic lipids. In some embodiments, molecules may be in the form of lipids, such as sterols, which are associated, either directly or indirectly, with an end of the oligonucleotide. In some embodiments, sterols (e.g., a cholesterol or a derivative thereof) are linked or attached to an end of an oligonucleotide, which may in turn associate with the exterior surface of a core (e.g., a hollow core) such that the oligonucleotides radiate outward from the core. In some embodiments, the sterol is linked or attached to a spacer, which is linked or attached to a sterol. The core may also be a solid, a semi-solid core or hollow core.

In some embodiments, the oligonucleotides of the SNA are associated with the core. An oligonucleotide that is associated with the core, such as a liposome core, may be linked or attached to (e.g., covalently) or associated with (e.g., non-covalently) the exterior surface of the core. In some embodiments, an oligonucleotide is associated with a core (e.g., liposome core) through a molecule, which, in some embodiments, results in a hydrophobic interaction. In some embodiments, the molecule is a sterol (e.g., cholesterol or cholesteryl ester). In some embodiments, when a sterol is associated with the exterior surface of the core, an oligonucleotide may be linked or attached to the sterol directly (e.g., covalently) or associated with the sterol indirectly (e.g., through a spacer). In some embodiments, when a lipid layer (e.g., lipid bilayer) forms the core (e.g., liposome core), the oligonucleotide is covalently linked or attached (e.g., directly) to one or more lipids in the lipid layer (e.g., lipid bilayer) or associated (e.g., indirectly) with one or more lipids in the lipid layer (e.g., lipid bilayer) through interactions (e.g., hydrophobic interactions) of a molecule (e.g., a sterol, such as a cholesterol or derivative thereof) linked or attached to the oligonucleotide, either directly or indirectly (e.g., through a spacer), with the lipid layer (e.g., lipid bilayer).

A CpG oligonucleotide, or an immunostimulatory CpG oligonucleotide refers to any CpG-containing oligonucleotide that is capable of activating an immune cell. At least the C of the CpG dinucleotide is typically unmethylated. Immunostimulatory CpG oligonucleotides are described in a number of issued patents and published patent applications, including U.S. Pat. Nos. 6,194,388; 6,207,646; 6,218,371; 6,239,116; 6,339,068; 6,406,705; and 6,429,199, which are incorporated by reference herein.

In some embodiments, a CpG oligonucleotide is 10, 11, 12, 13, 14, 15, 16, 17, 18, 19, 20, 21, 22, 23, 24, 25, 26, 27, 28, 29, 30, 31, 32, 33, 34, 35 or more than 35 nucleotides in length. In some embodiments, the CpG oligonucleotide is single-stranded. In some embodiments, the CpG oligonucleotide is hybridized to a second oligonucleotide (e.g., a second CpG oligonucleotide) and forms a double-stranded oligonucleotide (e.g., a double-stranded CpG oligonucleotide). In some embodiments, the CpG oligonucleotide is not hybridized to a second oligonucleotide (e.g., a second CpG oligonucleotide) and does not form a double-stranded oligonucleotide (e.g., is not a double-stranded CpG oligonucleotide).

In some embodiments, at least one oligonucleotide (e.g., a CpG oligonucleotide) of an SNA has its 5′-terminus exposed on the exterior surface away from the core. In some embodiments, all of the oligonucleotides (e.g., CpG oligonucleotides) of an SNA have their 5′-termini exposed on the exterior surface away from the core. In some embodiments, at least one oligonucleotide (e.g., a CpG oligonucleotide) of an SNA has its 3′-terminus exposed on the exterior surface away from the core. In some embodiments, all of the oligonucleotides (e.g., CpG oligonucleotides) of an SNA have their 3′-termini exposed on the exterior surface (away from the core). In some embodiments, the SNA does not include an oligonucleotide inside the core (e.g., liposome core).

In some embodiments, two or more of the oligonucleotides (e.g., CpG oligonucleotides) of an SNA are crosslinked. In some embodiments, all of the oligonucleotides (e.g., CpG oligonucleotides) of an SNA are crosslinked. In some embodiments, the oligonucleotides (e.g., CpG oligonucleotides) of an SNA are not crosslinked.

In some embodiments, at least one of the oligonucleotides (e.g., CpG oligonucleotides) of an SNA includes a substitution and/or modification. In some embodiments, the substitution and/or modification is in one or more nucleobases and/or sugars. For example, in some embodiments an oligonucleotide (e.g., CpG oligonucleotide) of an SNA includes nucleic acids having backbone sugars that are covalently attached to low molecular weight organic groups other than a hydroxyl group at the 2′ position and other than a phosphate group or hydroxyl group at the 5′ position. Thus, in some embodiments, a substituted or modified oligonucleotide (e.g., CpG oligonucleotide) of an SNA includes a 2′-O-alkylated ribose group. In some embodiments, a modified oligonucleotide (e.g., CpG oligonucleotide) of an SNA includes sugars such as hexose, 2′-F hexose, 2′-amino ribose, constrained ethyl (CEt), locked nucleic acid (LNA), arabinose or 2′-fluoroarabinose instead of ribose. Thus, in some embodiments, an oligonucleotide (e.g., CpG oligonucleotide) of an SNA is heterogeneous in backbone composition thereby containing any possible combination of polymer units linked together such as peptide-nucleic acids (which have an amino acid backbone with nucleic acid bases).

Modifications include, but are not limited to, for example, (a) end modifications, e.g., 5′ end modifications (phosphorylation, dephosphorylation, conjugation, inverted linkages, etc.) and 3′ end modifications (conjugation, DNA nucleotides, inverted linkages, etc.); (b) base modifications, e.g., replacement with modified bases, stabilizing bases, destabilizing bases, bases that base pair with an expanded repertoire of partners, or conjugated bases; (c) sugar modifications (e.g., at the 2′ position or 4′ position) or replacement of the sugar; as well as (d) internucleotide linkage modifications, including modification or replacement of the phosphodiester linkages (for example, with phosphorothioate linkages).

In some embodiments, one or more oligonucleotides (e.g., CpG oligonucleotides) of an SNA comprise at least one nucleotide in L-conformation. In some embodiments, the oligonucleotides (e.g., CpG oligonucleotides) of an SNA do not comprise a nucleotide in L-conformation. In some embodiments, none of the oligonucleotides (e.g., CpG oligonucleotides) of an SNA comprise nucleotides in L-conformation. In some embodiments, all of the oligonucleotides (e.g., CpG oligonucleotides) of an SNA consist of nucleotides in D-conformation.

In some embodiments, an oligonucleotide (e.g., CpG oligonucleotide) of an SNA comprises a modified backbone. In some embodiments, the modified backbone comprises modified internucleotide linkages. In some embodiments, at least one of the oligonucleotides (e.g., CpG oligonucleotides) of an SNA comprises at least one phosphorothioate internucleotide linkage. In some embodiments, each oligonucleotide (e.g., each CpG oligonucleotide) of an SNA comprises at least one phosphorothioate internucleotide linkage. In some embodiments, each internucleotide linkage of at least one of the oligonucleotides (e.g., CpG oligonucleotides) of an SNA is a phosphorothioate internucleotide linkage. In some embodiments, each internucleotide linkage of each of the oligonucleotides (e.g., CpG oligonucleotides) of an SNA is a phosphorothioate internucleotide linkage.

Non-limiting examples of modified internucleotide linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

In some embodiments, the SNA comprises a TLR agonist. A TLR agonist, as used herein is a nucleic acid molecule or oligonucleotide that interacts with and stimulates the activity of a TLR. The SNA, in some embodiments, is a TLR9-targeted SNA. In some embodiments, the SNA acts as a TLR9 agonist. In some embodiments, the oligonucleotides of an SNA comprise a TLR9 agonist or comprise or consist of TLR9 agonists. In some embodiments, the SNA comprises CpG oligonucleotides that are TLR9 agonists, and is referred to herein as an SNA of CpG oligonucleotides. In some embodiments, the TLR9 agonist is an oligonucleotide that comprises the nucleotide sequence of CpG-7909 (5′-TCGTCGTTTTGTCGTTTTGTCGTT-3′ (SEQ ID NO: 1)). In some embodiments, the oligonucleotide comprises one or more phosphorothioate bonds, internucleotide linkages or internucleoside linkages. In some embodiments, all the bases in the oligonucleotide are linked via phosphorothioate bonds, internucleotide linkages or internucleoside linkages or the oligonucleotide has a phosphorothioate backbone.

In some embodiments, an oligonucleotide disclosed herein, such as a CpG oligonucleotide, comprises a spacer. As disclosed herein, when a CpG oligonucleotide comprises a spacer, the CpG oligonucleotide is attached to a spacer. In some embodiments, the CpG oligonucleotide is attached to a spacer at its 3′-end, at its 5′-end, or at both its 3′- and 5′-ends. In some embodiments, the CpG oligonucleotide is attached to the spacer through a covalent bond. In some embodiments, the spacer is or comprises oligoethylene glycol. In some embodiments, the oligoethylene glycol is a hexaethylene glycol (HEG). In some embodiments, the spacer comprises two consecutive HEGs. In some embodiments, the oligoethylene glycol is triethylene glycol (TEG). In some embodiments, the spacer comprises or consists of two HEG molecules and a TEG. In some embodiments, the spacer comprises or consists of a HEG, a TEG, a HEG and a TEG, or a combination thereof. In some embodiments, the spacer does not comprise or consist of an oligonucleotide. In some embodiments, the spacer does not comprise a nucleobase. In some embodiments, the oligonucleotide further comprises a hydrophobic group, such as a sterol. In some embodiments, the hydrophobic group is a cholesterol or a cholesteryl ester.

In some embodiments, the oligonucleotide comprises or consists of the sequence 5′-TCGTCGTTTTGTCGTTTTGTCGTT-3′ (SEQ ID NO: 1). In some embodiments, the oligonucleotide comprises or consists of the sequence:

(SEQ ID NO: 1)
5′-T*C*G*T*C*G*T*T*T*T*G*T*C*G*T*T*T*T*G*T*C*
G*T*T-3′/HEG/HEG/TEG Cholesteryl Ester/.

The abbreviations used for this sequence are shown below:

| Abbreviation | Chemical Name |
|---|---|
| C | 2′-deoxy-P-cytidylyl |
| G | 2′-deoxy-P-guanylyl |
| T | 2′-deoxy-P-thymidylyl |
| HEG | Hexa(ethylene glycol)phosphodiester |
| TEG Cholesteryl | (N-cholestery1-3-aminopropyl)-triethyleneglycol- |
| Ester | glyceryl-1-O-phosphodiester |
| * | Phosphorothioate internucleotide linkage |

In some embodiments, the oligonucleotide that comprises or consists of the sequence 5′-T*C*G*T*C*G*T*T*T*T*G*T*C*G*T*T*T*T*G*T*C*G*T*T-3′/HEG/HEG/TEG Cholesteryl Ester/ (SEQ ID NO: 1) is in an SNA, which is also referred to herein as CpG-7909-SNA.

In some embodiments, an SNA disclosed herein, such as CpG-7909-SNA, is formulated in DOPC and in a phosphate buffered saline (PBS) buffer that acts as a pH and osmolality modifier (solvent).

An oligonucleotide (e.g., a CpG oligonucleotide) disclosed herein may be positioned on the exterior surface of the core. In some embodiments, at least or about 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 oligonucleotides (e.g., CpG oligonucleotides) or any range combination thereof are on the exterior surface of a liposome core. In some embodiments, between about 1 and about 100, about 5 and about 100, about 10 and about 100, about 15 and about 100, about 20 and about 100, about 25 and about 100, about 30 and about 100, about 25 and about 90, about 25 and about 85, about 25 and about 80, about 25 and about 75, about 25 and about 70, about 25 and about 65, about 25 and about 60, about 25 and about 55, about 25 and about 50, about 25 and about 45, about 25 and about 40, about 25 and about 33, about 25 and about 30 oligonucleotides (e.g., CpG oligonucleotides), or any range or combination thereof, are on the exterior surface of a liposome core.

An SNA (e.g., an SNA of CpG oligonucleotides) disclosed herein may include any number of oligonucleotides. In some embodiments, an SNA includes at least or about 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 oligonucleotides (e.g., CpG oligonucleotides) or any range combination thereof. In some embodiments, an SNA includes between about 1 and about 100, about 5 and about 100, about 10 and about 100, about 15 and about 100, about and about 100, about 25 and about 100, about 30 and about 100, about 25 and about 90, about 25 and about 85, about 25 and about 80, about 25 and about 75, about 25 and about 70, about 25 and about 65, about 25 and about 60, about 25 and about 55, about 25 and about 50, about 25 and about 45, about 25 and about 40, about 25 and about 33, about 25 and about 30 oligonucleotides (e.g., CpG oligonucleotides), or any range or combination thereof. In some embodiments, an SNA includes between about 25 and about 45 oligonucleotides (e.g., CpG oligonucleotides). The oligonucleotides may be positioned on the exterior surface of the SNA core (e.g., liposome core), may be attached to the surface of the SNA core (e.g., liposome core), or may be linked to the surface of the SNA core (e.g., liposome core) through a spacer.

In some embodiments, the SNA includes a neutral lipid. The neutral lipid may be, for example, 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), or any related phosphatidylcholine or neutral lipids available from commercial vendors.

In some embodiments, the immune checkpoint inhibitor is a molecule such as a monoclonal antibody, a humanized antibody, a fully human antibody, a fusion protein or a combination thereof, or a small molecule. For instance, the immune checkpoint inhibitor inhibits a checkpoint protein which may be programmed cell death protein-1 (PD-1). In some embodiments, the immune checkpoint inhibitor inhibits or decreases the activity of a PD-1 or one or more of its ligands. The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and inhibits it. Cancer cells take advantage of this system by driving high levels of expression of PD-L1 on their surface. This allows them to gain control of the PD-1 pathway and switch off T cells expressing PD-1 that may enter the tumor microenvironment, thus suppressing the anticancer immune response.

In some embodiments, the immune checkpoint inhibitor is an antibody that targets PD-1. In some embodiments, the antibody is pembrolizumab (formerly MK-3475 and lambrolizumab; KEYTRUDA®).

The immune checkpoint inhibitor may be a molecule such as a monoclonal antibody, a humanized antibody, a fully human antibody, a fusion protein or a combination thereof or a small molecule. For instance, the immune checkpoint inhibitor inhibits a checkpoint protein which may be CTLA-4, PD-L1, PDL2, PD-1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. Ligands of checkpoint proteins include but are not limited to CTLA-4, PD-L1, PDL2, PD-1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands.

In some embodiments, the anti-PD-1 antibody is BMS-936558 (also known as Opdivo; nivolumab).

In other embodiments, the anti-CTLA-4 antibody is ipilimumab (trade name Yervoy, formerly known as MDX-010 and MDX-101).

In some embodiments, the anti-PD-1 antibody is REGN-2810 (cemiplimab).

In some embodiments, the anti-PD-1 antibody is PDR001 (spartalizumab).

In some embodiments, the anti-PD-1 antibody is retifanlimab (INCMGA 0012).

In some embodiments, the anti-PD-1 antibody is ABBV-181.

In some embodiments, the immune checkpoint inhibitor is an anti-PD-L1 antibody.

In some embodiments, the anti-PD-L1 antibody is MPDL3280A (also known as Tecentriq; atezolizumab).

In some embodiments, the anti-PD-L1 antibody is MSB0010718C (avelumab).

In some embodiments, the anti-PD-L1 antibody is MEDI4736 (durvalumab).

In vitro experiments show that an SNA, such as an SNA of CpG oligonucleotides, exhibits cellular uptake and activity that is greater than the corresponding linear oligonucleotide and demonstrates specificity for targets, such as TLR9. In some embodiments, SNA of CpG oligonucleotides have the potential to treat a variety of tumor types. This is demonstrated, in non-limiting embodiments, in decreased tumor volume and increased median survival compared to a reference level, baseline level or control. In some embodiments, SNAs (e.g., SNA of CpG oligonucleotides) show anti-tumor activity after administration by any of three routes of administration: SC, IT, intravenous (IV) injection, or IV infusion routes of administration. Finally, across a variety of tumor models and routes of administration, an SNA of CpG oligonucleotides can be combined with immune checkpoint inhibitor (CPI) antibodies (e.g., anti-PD-1 or anti-PD-L1) to produce enhanced anti-tumor effects compared with either agent alone. In some embodiments, an SNA (e.g., an SNA of a CpG oligonucleotide, such as CpG-7909) can be combined with an agonist of a co-stimulatory molecule (e.g., an agonist of a TNFRSF member) to induce a desired response to treat a disease (e.g., cancer).

Anti-PD-1 antibody treatment blocks the interaction of PD-1 and the upregulated PD-L1 on tumor-targeting T cells and tumor cells, respectively, to ensure that the anti-tumor T cells are not inactivated. However, anti-PD-1 antibody refractory/non-responding patients may not have tumor specific T cells to act on the cancer tissue, or their tumor specific T cells may be suppressed through a variety of mechanisms. TLR9 agonism within the tumor is designed to stimulate tumor cell killing by natural killer (NK) cells, stimulate tumor antigen generation, and eventually stimulate activation of tumor specific cytotoxic T cells, thus addressing the lack of tumor-specific T cells in the patients. In patients where tumor specific T cells are already present, CPIs may work as a monotherapy, but the response can be improved by pushing the immune system further by its activation with a TLR9 agonist, as disclosed herein.

In some embodiments, an SNA (e.g., an SNA of a CpG oligonucleotide, such as CpG-7909) and an agonist of a co-stimulatory molecule (e.g., an agonist of a TNFRSF member) is combined with an immune checkpoint inhibitor (e.g., an immune checkpoint inhibitor antibody, such as an anti-PD-1 antibody) to treat a disease.

Agonists of co-stimulatory molecules, such as antibodies that activate co-stimulatory molecules, as disclosed herein, promote T cell division and survival, facilitating clonal expansion of effector and memory T cell populations specific to an antigen. Certain agonists can additionally suppress the differentiation and/or activity of regulatory T cells (Treg), which can further improve T cell expansion and can increase cytotoxic T cell activity. Co-stimulation can also regulate cytokine production from various immune cell populations, including T cells, antigen-presenting cells, NK cells and NKT cells, and can modulate cytokine receptor signaling. See, e.g., Magee, et al., Am. J. Transplant (2012) 12(10):2588-2600; Sharpe, Immunol. Rev. (2009) 229(1):5-11; Croft, et al., Immunol. Rev. (2009) 229(1):173-191. As such, agonism of co-stimulatory molecules and TLR9 agonism can improve immune responses, including anti-tumor immune responses, through complementary mechanisms.

As disclosed herein, an agonist of a TNFRSF member refers to any molecule, compound, drug, or other pharmaceutical composition which interacts with a member of the tumor necrosis factor receptor superfamily (TNFRSF) in a way that results in activation of the TNFRSF member or increases its activity. For example, an agonist of a TNFRSF member may bind to the TNFRSF member and activate signaling downstream of the TNFRSF member. The terms “agonist of a TNFRSF member” and “TNFRSF member agonist” are used interchangeably herein.

In some embodiments, the agonist of a TNFRSF member is a molecule such as a monoclonal antibody, a humanized antibody, a fully human antibody, a fusion protein or a combination thereof, a peptide, an oligonucleotide, an aptamer, or a small molecule. For instance, the agonist of a TNFRSF member interacts with a TNFRSF member, which in some embodiments is OX40. In some embodiments, the agonist of a TNFRSF member activates or increases the activity of an immune co-stimulatory molecule, such as OX40. In some embodiments, the TNFRSF member agonist activates the TNFRSF member such that the immune cell or immune cells on which the TNFRSF member is activated are stimulated. In some embodiments, the TNFRSF member stimulates an immune response in the subject or the cells to which it is administered.

In some embodiments, the TNFRSF member is TNFR1, TNFR2, Lymphotoxin beta receptor, OX40, CD40, Fas receptor, Decoy receptor 3, CD27, CD30, CD137 (4-1BB), Death receptor 4, Death receptor 5, Decoy receptor 1, Decoy receptor 2, RANK, Osteoprogerin, TWEAK receptor (CD266), TACI, BAFF receptor, Herpesvirus entry mediator (HVEM), Nerve growth factor receptor (CD271), B-cell maturation antigen, Glucocorticoid-induced TNFR-related protein (GITR), TROY, Death receptor 6, Death receptor 3, or Ectodysplasin A2 receptor. In some embodiments, the TNFRSF member is OX40.

In some embodiments, interaction of an agonist with the TNFRSF member activates signaling downstream of the TNFRSF member. In some embodiments, interaction of an agonist with the TNFRSF member activates signaling downstream of OX40. In some embodiments, the agonist of a TNFRSF member is an OX40 agonist. In some embodiments, the OX40 agonist is an anti-OX40 antibody. In some embodiments, the agonist of a TNFRSF member is an anti-OX40 antibody. In some embodiments, the anti-OX40 antibody is INCAGN1949. INCAGN1949 is an antibody which binds specifically to OX40 in a way that activates OX40 signaling. In some embodiments, the anti-OX40 antibody is PF-04518600. In some embodiments, the anti-OX40 antibody is GSK3174998. In some embodiments, the anti-OX40 antibody is BMS-986178. In some embodiments, the anti-OX40 antibody is tavolixizumab (MEDI 0562). In some embodiments, the anti-OX40 antibody is pogalizumab (MOXR0916/RG7888). In some embodiments, the anti-OX40 antibody is ABBV-368.

In some embodiments, the TNFRSF member is 4-1BB (CD137). In some embodiments, the agonist of a TNFRSF member is an agonist of 4-1BB. In some embodiments, the agonist of 4-1BB is an anti-4-1BB antibody. In some embodiments, the agonist of a TNFRSF member is an anti-4-1BB antibody. In some embodiments, the anti-4-1BB antibody is utomilumab (PF-05082566).

In some embodiments, the method for treating a disease (e.g., cancer) comprises administering a dose of two or more of an agonist of a TNFRSF member, an immune checkpoint inhibitor, and an SNA comprising a CpG oligonucleotide linked through a spacer to an exterior surface of a liposome core having a diameter of less than 40 nm or less than about 40 nm.

Taken together, these properties make SNAs (e.g., SNAs of CpG oligonucleotides) effective for a therapeutic (e.g., immune-oncology-based therapeutic) and an exceptional combination partner for a second therapeutic (e.g., a second immune-mediated oncology therapeutic), such as immune checkpoint inhibitor antibodies) and/or a third therapeutic (e.g., agonist of a co-stimulatory molecule, a VEGF antibody). An immune checkpoint inhibitor includes, but is not limited to, anti-PD-1 antibodies (e.g., retifanlimab (INCMGA 0012), pembrolizumab, cemiplimab and ABBV-181), and anti-PD-L1 antibodies (e.g., avelumab), or anti-PD-1 antibodies and anti-PD-L1 antibodies. In some embodiments, agonists of co-stimulatory molecules include but are not limited to agonists of TNFRSF members, immunoglobulin (Ig) superfamily members, and T cell Ig and mucin (TIM) domain family members.

Disclosed herein are, in some aspects, different doses, routes of administration and orders of administration that unexpectedly result in the successful treatment of diseases (e.g., cancer) in a subject, such as a human.

Accordingly, in some embodiments, methods for treating a disease (e.g., cancer) are disclosed herein. In some embodiments, the method for treating a disease (e.g., cancer) comprises administering to a subject an SNA and an agonist of a TNFRSF member, or an SNA, an agonist of a TNFRSF member and an immune checkpoint inhibitor. In some embodiments, the SNA comprises a core having an oligonucleotide shell comprised of CpG oligonucleotides positioned on the exterior of the core, wherein the SNA is administered to the subject at a fixed dose of at least about 2 mg. In some embodiments, the fixed dose is administered to one solid tumor or to one tumor lesion in the subject or the fixed dose is divided among two or more solid tumors or two or more tumor lesions in the subject.

Accordingly, in some embodiments, methods for treating cancer (e.g., BCC, such as locally advanced BCC, metastatic BCC, and/or superficial lesions in BCC, melanoma or liver cancer, such as HCC) are disclosed herein. In some embodiments, the method for treating cancer (e.g., BCC, such as locally advanced BCC, metastatic BCC, and/or superficial lesions in BCC, melanoma or liver cancer, such as HCC) comprises administering to a subject an SNA and an immune checkpoint inhibitor, wherein the SNA comprises a core having an oligonucleotide shell comprised of CpG oligonucleotides positioned on the exterior of the core, wherein the SNA is administered to the subject at a fixed dose of at least about 2 mg. In some embodiments, the fixed dose is administered to one solid tumor or to one tumor lesion in the subject or the fixed dose is divided among two or more solid tumors or two or more tumor lesions in the subject. In some embodiments, the SNA is administered within 24 hours of administration of the immune checkpoint inhibitor in any order (e.g., the SNA is administered first within the 24-hour period or the immune checkpoint inhibitor is administered first with the 24-hour period) to treat the cancer (e.g., BCC, such as locally advanced BCC, metastatic BCC, and/or superficial lesions in BCC, melanoma or liver cancer, such as HCC) in the subject.

In some embodiments, the SNA is administered within 24 hours of administration of one or both of the immune checkpoint inhibitor and the agonist of a TNFRSF member in any order (e.g., the SNA is administered first within the 24-hour period, the agonist of a TNFRSF member is administered first within the 24-hour period, or the immune checkpoint inhibitor is administered first with the 24-hour period) to treat the disease (e.g., cancer) in the subject. In some embodiments, the SNA is administered within 12 hours of administration of one or both of the immune checkpoint inhibitor and the agonist of a TNFRSF member in any order (e.g., the SNA is administered first within the 12-hour period, the agonist of a TNFRSF member is administered first within the 12-hour period, or the immune checkpoint inhibitor is administered first with the 12-hour period) to treat the disease (e.g., cancer) in the subject.

In some embodiments, the method for treating a disease (e.g., cancer) comprises administering to a subject an agonist of a TNFRSF member and an SNA (e.g., an SNA of CpG oligonucleotides disclosed herein), wherein the SNA comprises a core and an oligonucleotide shell comprised of CpG oligonucleotides positioned on the exterior of the core.

In some embodiments, the method for treating a disease (e.g., cancer) comprises administering to a subject an agonist of a TNFRSF member, an immune checkpoint inhibitor, and an SNA (e.g., an SNA of CpG oligonucleotides) disclosed herein, wherein the SNA comprises a core and an oligonucleotide shell comprised of CpG oligonucleotides positioned on the exterior of the core.

In some embodiments, the method for treating a disease (e.g., cancer) comprises administering to a subject an additional therapeutic agent, such as a VEGF antibody. In some embodiments, the VEGF antibody is bevacizumab (Avastin®). In some embodiments, the VEGF antibody is administered at a dose of or about 15 mg/kg.

In some embodiments, the SNA is administered once a week, once every three weeks, or once a week followed by once every three weeks. In some embodiments, the SNA is administered once a week for up to 12 weeks, followed by once every three weeks. In some embodiments, the SNA is administered once a week for nine weeks, followed by once every three weeks. In some embodiments, the SNA is administered at a dose of between 1 mg and 50 mg.

In some embodiments, the agonist of a TNFRSF member is administered on a different schedule than the SNA. In some embodiments, the agonist of a TNFRSF member is administered on the same schedule as the SNA. In some embodiments, the agonist of a TNFRSF member is administered on the same schedule as the SNA and then on a different schedule, or on a different schedule and then the same schedule. In some embodiments, an immune checkpoint inhibitor is administered on the same schedule as the SNA or the agonist of a TNFRSF member, or is administered on a different schedule as the SNA or the agonist of a TNFRSF member.

In some embodiments, a dose of an SNA, an immune checkpoint inhibitor, the combination of an SNA and an immune checkpoint inhibitor disclosed herein, or the combination of an SNA, an agonist of a TNFRSF member and an immune checkpoint inhibitor disclosed herein refers to a dose or dose combination that inhibits tumor cell growth or tumor growth by at least or at least about 5%, at least or at least about 10%, at least or at least about 15%, at least or at least about 20%, at least or at least about 30%, at least or at least about 35%, at least or at least about 40%, at least or at least about 45%, at least or at least about 50%, at least or at least about 55%, at least or at least about 60%, at least or at least about 65%, at least or at least about 70%, at least or at least about 75%, at least or at least about 80%, at least or at least about 85%, at least or at least about 90%, at least or at least about 95%, at least or at least about 99%, relative to a reference level.

In some embodiments, a “compound” refers to an SNA, an agonist of a TNFRSF member, or an immune checkpoint inhibitor.

In some embodiments, a dose of a compound disclosed herein, such as a dose of an SNA, the combination of an SNA and an agonist of a TNFRSF member, the combination of an SNA, an agonist of a TNFRSF member and an immune checkpoint inhibitor disclosed herein, or the combination of an SNA with an immune checkpoint inhibitor disclosed herein can decrease the number of solid tumors or tumor lesions, decrease the tumor size of one or more of the solid tumors or tumor lesions, or otherwise ameliorate symptoms associated with cancer (e.g., BCC, such as locally advanced BCC, metastatic BCC, and/or superficial lesions in BCC, melanoma or liver cancer, such as HCC) in a subject. In some embodiments, the tumor size is decreased by at least or at least about 5%, at least or at least about 10%, at least or at least about 15%, at least or at least about 20%, at least or at least about 30%, at least or at least about 35%, at least or at least about 40%, at least or at least about 45%, at least or at least about 50%, at least or at least about 55%, at least or at least about 60%, at least or at least about 65%, at least or at least about 70%, at least or at least about 75%, at least or at least about 80%, at least or at least about 85%, at least or at least about 90%, at least or at least about 95%, at least or at least about 99%, relative to a reference tumor size.

The terms “baseline tumor size” or “reference tumor size,” as disclosed herein, refers to the size of a solid tumor or a tumor lesion, refers to an average of two or more of solid tumors or two or more tumor lesions, or refers to a sum of diameters of two or more of solid tumors or two or more tumor lesions, in a subject with cancer (e.g., BCC, such as locally advanced BCC, metastatic BCC, and/or superficial lesions in BCC, melanoma or liver cancer, such as HCC) prior to administration of a therapeutic dose of an SNA (e.g., an SNA disclosed herein) only, prior to administration of a therapeutic dose of an immune checkpoint inhibitor (e.g., an immune checkpoint inhibitor disclosed herein) only, prior to administration of a dose of an agonist of a TNFRSF member (e.g., an agonist of a TNFRSF member disclosed herein), or prior to administration of a combination of two or more of a dose of an SNA (e.g., an SNA disclosed herein), a dose of an immune checkpoint inhibitor (e.g., an immune checkpoint inhibitor disclosed herein) and a dose of an agonist of a TNFRSF member (e.g., an agonist of a TNFRSF member disclosed herein). In some embodiments, the baseline tumor size or reference tumor size refers to the baseline sum of diameters of solid tumors or tumor lesions in a subject with cancer (e.g., BCC, such as locally advanced BCC, metastatic BCC, and/or superficial lesions in BCC, melanoma or liver cancer, such as HCC). In some embodiments, the baseline tumor size or reference tumor size refers to the tumor size prior to initiation of treatment with an SNA, an immune checkpoint inhibitor, an agonist of a TNFRSF member, or a combination of two or more of an SNA, an immune checkpoint inhibitor and an agonist of a TNFRSF member. In some embodiments, the baseline tumor size or reference tumor size refers to the tumor size prior to a single treatment with an SNA, an immune checkpoint inhibitor, an agonist of a TNFRSF member, or a combination of two or more of an SNA, an immune checkpoint inhibitor and a TNFRSF member.

In some embodiments, amelioration of symptoms associated with cancer (e.g., BCC, such as locally advanced BCC, metastatic BCC, and/or superficial lesions in BCC, melanoma or liver cancer, such as HCC) refers to amelioration of pain.

In some embodiments, administration of a dose results in a certain tumor response, as measured by the Response Evaluation Criteria in Solid Tumors (RECIST) criteria. The RECIST criteria, known to those of ordinary skill in the art, are used to determine objective tumor response for target lesions. (See e.g., Eisenhauer et al. Eur J Cancer (2009) 45(2):228-47). In some embodiments, administration of a dose of (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, results in a complete response, as measured by the RECIST criteria.

In some embodiments, administration of (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, results in a complete response.

In some embodiments, a “complete response,” as defined by the RECIST criteria and as used herein, refers to the disappearance of all target lesions. In some embodiments, a complete response refers to the disappearance of one or more target lesions (e.g., the disappearance of at least one target lesion, at least two target lesions, at least three target lesions, at least four target lesions, at least five target lesions, at least six target lesions, at least seven target lesions, at least eight target lesions, at least nine target lesions, at least 10 target lesions, at least 11 target lesions, or at least 12 target lesions, etc., or any range or combination thereof). In some embodiments, in a complete response, pathological lymph nodes (whether target or non-target) have reduction in short axis to less than 10 mm.

In some embodiments, administration of (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, results in a “partial response,” as measured by the RECIST criteria and as used herein.

In some embodiments, a “partial response”, as defined by the RECIST criteria and as used herein, refers to at least a 30% decrease in the sum of diameters of target lesions, taking as reference the baseline sum diameters.

In some embodiments, administration of an SNA, an immune checkpoint inhibitor, and an agonist of a TNFRSF member, as disclosed herein, results in a partial response.

In some embodiments, a partial response refers to a decrease in the diameter of at least one target lesion (e.g., a decrease in the diameter of one target lesion, at least two target lesions, at least three target lesions, at least four target lesions, at least five target lesions, at least six target lesions, at least seven target lesions, at least eight target lesions, at least nine target lesions, at least 10 target lesions, at least 11 target lesions, or at least 12 target lesions, etc., or any range or combination thereof) or a decrease in the sum of the diameters of two or more target lesions (e.g., a decrease in the sum of the diameters of at least two target lesions, at least three target lesions, at least four target lesions, at least five target lesions, at least six target lesions, at least seven target lesions, at least eight target lesions, at least nine target lesions, at least 10 target lesions, at least 11 target lesions, or at least 12 target lesions, etc., or any range or combination thereof) by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99%, relative to a baseline level or a reference level.

In some embodiments, a “baseline level” or “reference level,” as disclosed herein, are used interchangeably to refer to a corresponding level in a subject with a disease (e.g., cancer) who has been administered: an SNA (e.g., an SNA disclosed herein) only; an agonist of a TNFRSF member (e.g., a TNFRSF member disclosed herein) only; an immune checkpoint inhibitor (e.g., an immune checkpoint inhibitor disclosed herein) only; an SNA (e.g., an SNA disclosed herein) and an immune checkpoint inhibitor (e.g., an immune checkpoint inhibitor disclosed herein) only; or has not been administered an SNA (e.g., an SNA disclosed herein), an agonist of a TNFRSF member (e.g., a TNFRSF member disclosed herein), and an immune checkpoint inhibitor (e.g., an immune checkpoint inhibitor disclosed herein). In some embodiments, a baseline level or reference level refers to a corresponding level in a subject without the corresponding disease (e.g., cancer). In some embodiments, a baseline level or reference level refers to a corresponding level in a subject without cancer. In some embodiments, a baseline level or reference level refers to a corresponding level in a subject before administration of a treatment disclosed herein, such as administration of: an SNA (e.g., an SNA disclosed herein); an agonist of a TNFRSF member (e.g., a TNFRSF member disclosed herein); an immune checkpoint inhibitor (e.g., an immune checkpoint inhibitor disclosed herein); an SNA (e.g., an SNA disclosed herein) and an immune checkpoint inhibitor (e.g., an immune checkpoint inhibitor disclosed herein); or an SNA (e.g., an SNA disclosed herein), an agonist of a TNFRSF member (e.g., a TNFRSF member disclosed herein), and an immune checkpoint inhibitor (e.g., an immune checkpoint inhibitor disclosed herein).

In some embodiments, baseline level or reference level are used interchangeably to refer to a corresponding level in a subject with cancer (e.g., BCC, such as locally advanced BCC, metastatic BCC, and/or superficial lesions in BCC, melanoma or liver cancer, such as HCC) that has been administered an SNA (e.g., an SNA disclosed herein) only, to refer to a corresponding level in a subject with cancer (e.g., BCC, such as locally advanced BCC, metastatic BCC, and/or superficial lesions in BCC, melanoma or liver cancer, such as HCC) that has been administered an immune checkpoint inhibitor (e.g., an immune checkpoint inhibitor disclosed herein) only, or to refer to a corresponding level in a subject with cancer (e.g., BCC, such as locally advanced BCC, metastatic BCC, and/or superficial lesions in BCC, melanoma or liver cancer, such as HCC) that has not been administered an SNA (e.g., an SNA disclosed herein) or an immune checkpoint inhibitor (e.g., an immune checkpoint inhibitor disclosed herein). In some embodiments, a baseline level or reference level refers to a corresponding level in a subject without the corresponding cancer (e.g., a subject without melanoma, Merkel cell carcinoma, cutaneous squamous cell carcinoma, liver cancer such as HCC, BCC, such as locally advanced BCC, metastatic BCC, and/or superficial lesions in BCC, head and neck squamous cell carcinoma, or mucosal melanoma). In some embodiments, a baseline level or reference level refers to a corresponding level in a subject without cancer (e.g., BCC, such as locally advanced BCC, metastatic BCC, and/or superficial lesions in BCC, melanoma or liver cancer, such as HCC). In some embodiments, a baseline level or reference level refers to a corresponding level in a subject a specified length of time prior to administration of an SNA, an agonist of a TNFRSF member, an immune checkpoint inhibitor, or a combination of two or more of an SNA, a TNFRSF member and an immune checkpoint inhibitor. For example, in some embodiments, a baseline level or reference level may in some embodiments refer to a level measured at a time corresponding to one therapeutic administration, to be used as a comparator for a level measured at a later time, which may in some embodiments correspond to a subsequent dose.

In some embodiments, a baseline level or reference level refers to a corresponding level in a subject (e.g., in a population of cells obtained from the subject) with a disease (e.g., cancer) who has been administered: an SNA (e.g., an SNA disclosed herein) only; an agonist of a TNFRSF member (e.g., a TNFRSF member disclosed herein) only; an immune checkpoint inhibitor (e.g., an immune checkpoint inhibitor disclosed herein) only; an SNA (e.g., an SNA disclosed herein) and an immune checkpoint inhibitor (e.g., an immune checkpoint inhibitor disclosed herein) only; or has not been administered an SNA (e.g., an SNA disclosed herein), an agonist of a TNFRSF member (e.g., a TNFRSF member disclosed herein), and an immune checkpoint inhibitor (e.g., an immune checkpoint inhibitor disclosed herein). In some embodiments, a baseline level or reference level refers to a corresponding level in a subject (e.g., in a population of cells obtained from the subject) without the corresponding disease (e.g., cancer). In some embodiments, a baseline level or reference level refers to a corresponding level in a subject (e.g., in a population of cells obtained from the subject) without cancer.

In some embodiments, a baseline level or reference level refers to a corresponding level in a population of cells obtained from a subject with cancer (e.g., BCC, such as locally advanced BCC, metastatic BCC, and/or superficial lesions in BCC, melanoma or liver cancer, such as HCC) that has been administered an SNA (e.g., an SNA disclosed herein) only, to refer to a corresponding level in a population of cells obtained from a subject with cancer that has been administered an immune checkpoint inhibitor (e.g., an immune checkpoint inhibitor disclosed herein) only, or to refer to a corresponding level in a population of cells obtained from a subject with cancer that has not been administered an SNA (e.g., an SNA disclosed herein) or an immune checkpoint inhibitor (e.g., an immune checkpoint inhibitor disclosed herein). In some embodiments, a baseline level or reference level refers to a corresponding level in a population of cells obtained from a subject without the corresponding cancer (e.g., a subject without melanoma, Merkel cell carcinoma, liver cancer such as HCC, BCC, such as locally advanced BCC, metastatic BCC, and/or superficial lesions in BCC, cutaneous squamous cell carcinoma, head and neck squamous cell carcinoma, or mucosal melanoma). In some embodiments, a baseline level or reference level refers to a corresponding level in a population of cells obtained from a subject without cancer (e.g., BCC, such as locally advanced BCC, metastatic BCC, and/or superficial lesions in BCC, melanoma or liver cancer, such as HCC). In some embodiments, the cancer (e.g., BCC, such as locally advanced BCC, metastatic BCC, and/or superficial lesions in BCC, melanoma or liver cancer, such as HCC) in the subject is progressive disease and administration of an SNA (e.g., a therapeutic dose of an SNA disclosed herein) or administration of an SNA in combination with an immune checkpoint inhibitor (e.g., a therapeutic dose of an SNA disclosed herein and a therapeutic dose of an immune checkpoint inhibitor disclosed herein) for the treatment of the cancer in the subject renders the cancer stable disease.

In some embodiments, the method for treating a cancer in a subject is progressive disease (PD) and administration of an SNA (e.g., a dose of an SNA disclosed herein) or administration of an SNA, an immune checkpoint inhibitor and an agonist of a TNFRSF member, each at a dose disclosed herein, for the treatment of the cancer in the subject, renders the cancer stable disease (SD).

In some embodiments, the cancer is SD for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks. In some embodiments, the cancer is SD for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months. In some embodiments, the cancer is SD for at least one year, at least two years, at least three years, at least four years, at least five years, at least six years, at least seven years, at least eight years, at least nine years, at least 10 years, at least 11 years or at least 12 years.

In some embodiments, a subject with cancer (e.g., BCC, such as locally advanced BCC, metastatic BCC, and/or superficial lesions in BCC, melanoma or liver cancer, such as HCC) has at least one target lesion, at least two target lesions, at least three target lesions, at least four target lesions or more and wherein administration of (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, decreases the diameter of at least one target lesion in the subject or decreases the sum of the diameters of two or more target lesions in the subject by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99% relative to a reference level. In some embodiments, administration of an SNA, an immune checkpoint inhibitor, and an agonist of a TNFRSF member, as disclosed herein, decreases the diameter of at least one target lesion in the subject by at least or about by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99% relative to a reference level. In some embodiments, administration of an SNA, an immune checkpoint inhibitor, and an agonist of a TNFRSF member, as disclosed herein, decreases the diameter of at least one target lesion in the subject or decreases the sum of the diameters of two or more target lesions in the subject by at least or about 20%.

In some embodiments, a subject with cancer (e.g., BCC, such as locally advanced BCC, metastatic BCC, and/or superficial lesions in BCC, melanoma or liver cancer, such as HCC) has at least one target lesion, at least two target lesions, at least three target lesions, at least four target lesions, or more and wherein administration of (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, results in partial response or results in complete response in at least one target lesion, at least two target lesions, at least three target lesions, at least four target lesions, or more in the subject with cancer. In some embodiments, administration of an SNA, an immune checkpoint inhibitor, and an agonist of a TNFRSF member, as disclosed herein, results in partial response or results in complete response in at least one target lesion.

In some embodiments administration of (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, results in partial response (e.g., overall partial response) or complete response (e.g., overall complete response) for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks in the subject. In some embodiments, administration of an SNA, an immune checkpoint inhibitor and an agonist of a TNFRSF member, as disclosed herein, results in partial response (e.g., overall partial response) or complete response (e.g., overall complete response) for at least two weeks.

In some embodiments, administration of (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, results in partial response or complete response for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months in the subject. In some embodiments, administration of an SNA, an immune checkpoint inhibitor and an agonist of a TNFRSF member, as disclosed herein, results in partial response or complete response for at least two months.

In some embodiments, administration of (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, results in partial response or complete response for at least one year, at least two years, at least three years, at least four years, at least five years, at least six years, at least seven years, at least eight years, at least nine years, at least 10 years, at least 11 years or at least 12 years in the subject.

In some embodiments, compounds, such as an SNA, an agonist of a TNFRSF member, and an immune checkpoint inhibitor disclosed herein are administered in adjuvant or neoadjuvant therapeutic modalities to treat a disease (e.g., cancer) in a subject. An adjuvant therapy refers to treatment with an anti-cancer agent (e.g., an SNA, an agonist of a TNFRSF member, or an immune checkpoint inhibitor disclosed herein) in addition to a different initial treatment, to increase the likelihood of successful treatment and/or to decrease the probability of cancer recurrence. In a non-limiting example, an adjuvant therapy for cancer refers to surgery followed by administration of an anti-cancer agent (e.g., an SNA, an agonist of a TNFRSF member, or an immune checkpoint inhibitor disclosed herein). A neoadjuvant therapy refers to a treatment with an anti-cancer agent (e.g., an SNA, an agonist of a TNFRSF member, or an immune checkpoint inhibitor disclosed herein) prior to a second treatment to shrink a tumor prior to the second treatment. In a non-limiting example, a neoadjuvant therapy for cancer may refer to administration of an anti-cancer agent (e.g., an SNA, an agonist of a TNFRSF member, or an immune checkpoint inhibitor disclosed herein) prior to surgery. In some embodiments, a neoadjuvant treatment increases the likelihood of successful or complete resection of a tumor during surgery, and/or decreases the likelihood of adverse outcomes during a surgery to remove tumor(s).

In some embodiments, the method for treating cancer (e.g., BCC, such as locally advanced BCC, metastatic BCC, and/or superficial lesions in BCC, melanoma or liver cancer, such as HCC) comprises administering a dose (e.g., therapeutic dose or fixed dose) of a spherical nucleic acid (SNA) comprising a CpG oligonucleotide linked through a spacer to an exterior surface of a liposome core having a diameter of less than about 40 nm and an immune checkpoint inhibitor, wherein the SNA is administered by IT injection or IT administration into multiple lesions at a dose of between about 16 mg and about 32 mg and the immune checkpoint inhibitor is administered by IV injection or IV infusion at a dose of between 180 and 220 mg, wherein the cancer is BCC (e.g., locally advanced BCC, metastatic BCC, and/or superficial BCC).

In some embodiments, the method for treating cancer (e.g., BCC, such as locally advanced BCC, metastatic BCC, and/or superficial lesions in BCC, melanoma or liver cancer, such as HCC) comprises administering to a subject a spherical nucleic acid (SNA) and an immune checkpoint inhibitor, wherein the SNA comprises a core having an oligonucleotide shell comprised of CpG oligonucleotides positioned on the exterior of the core, wherein the SNA is administered at a dose of between about 16 mg to about 32 mg every three weeks, wherein the immune checkpoint inhibitor is administered at a dose of between 180 and 220 mg every three weeks, wherein the SNA is administered within 24 hours of the administration of the immune checkpoint inhibitor, and wherein the SNA and the immune checkpoint inhibitor are administered through different routes of administration to treat the cancer in the subject, wherein the cancer is BCC.

In some embodiments, the SNA is administered at a dose (e.g., therapeutic dose or fixed dose) of or about 16 mg. In some embodiments, the SNA is administered at a dose of or about 32 mg. In some embodiments, the SNA is administered at a dose of or about 16 mg and the immune checkpoint inhibitor is administered at a dose of or about 200 mg. In some embodiments, the SNA is administered at a dose of or about 32 mg and the immune checkpoint inhibitor is administered at a dose of or about 200 mg. In some embodiments, the SNA is administered at a dose of or about 16 mg. In some embodiments, the SNA is administered at a dose of or about 32 mg. In some embodiments, the SNA is administered at a dose of or about 16 mg and the immune checkpoint inhibitor is administered at a dose of or about 350 mg. In some embodiments, the SNA is administered at a dose of or about 32 mg and the immune checkpoint inhibitor is administered at a dose of or about 350 mg. In some embodiments, the immune checkpoint inhibitor is an anti-PD-1 antibody. In some embodiments, the immune checkpoint inhibitor is pembrolizumab or cemiplimab. In some embodiments, the immune checkpoint inhibitor is cemiplimab and the cancer is BCC, such as locally advanced BCC, metastatic BCC, and/or superficial lesions in BCC. In some embodiments, the immune checkpoint inhibitor is an anti-PD-L1 antibody. In some embodiments, the immune checkpoint inhibitor is avelumab administered at a dose of or about 800 mg and the cancer is Merkel cell carcinoma.

As disclosed herein, the dose (e.g., therapeutic dose or fixed dose) as it relates to administration of an SNA disclosed herein (e.g., without limitation the phrases “SNA is administered at a dose,” “SNA is administered to the subject at a fixed dose,” “total dose of an SNA disclosed herein and/or the total volume of an SNA disclosed herein is administered,” or “SNA dose administered,” etc.) refers to the total weight or total mass of the active agent (i.e., total weight or total mass of CpG oligonucleotides) that are part of the SNA and is being administered to the subject (e.g., a subject with cancer).

In some embodiments, administration of the SNA is at a dose (e.g., therapeutic dose or fixed dose) between 0.1 mg and 10 mg, between 0.2 mg and 10 mg, between 0.3 mg and 10 mg, between 0.4 mg and 10 mg, between 0.5 mg and 10 mg, between 0.6 mg and 10 mg, between 0.7 mg and 10 mg, between 0.8 mg and 10 mg, between 0.9 mg and 10 mg, between 1 mg and 10 mg, between 1 mg and 1000 mg, between 1 mg and 900 mg, between 1 mg and 800 mg, between 1 mg and 700 mg, between 1 mg and 600 mg, between 1 mg and 500 mg, between 1 mg and 450 mg, between 1 mg and 400 mg, between 1 mg and 350 mg, between 1 mg and 300 mg, between 1 mg and 250 mg, between 1 mg and 200 mg, between 1 mg and 150 mg, between 1 mg and 100 mg, between 1 mg and 90 mg, between 1 mg and 80 mg, between 1 mg and 70 mg, between 1 mg and 60 mg, between 1 mg and 60 mg, between 1 mg and 50 mg, between 1 mg and 49 mg, between 1 mg and 48 mg, between 1 mg and 47 mg, between 1 mg and 46 mg, between 1 mg and 45 mg, between 1 mg and 44 mg, between 1 mg and 43 mg, between 1 mg and 42 mg, between 1 mg and 41 mg, between 1 mg and 40 mg, between 1 mg and 39 mg, between 1 mg and 38 mg, between 1 mg and 37 mg, between 1 mg and 36 mg, between 1 mg and 35 mg, between 1 mg and 34 mg, between 1 mg and 33 mg, between 1 mg and 32 mg, between 1 mg and 31 mg, between 1 mg and 30 mg, between 1 mg and 29 mg, between 1 mg in 28 mg, between 1 mg and 27 mg, between 1 mg and 26 mg, between 1 mg and 25 mg, between 1 mg and 24 mg, between 1 mg and 23 mg, between 1 mg and 22 mg, between 1 mg and 21 mg, between 1 mg and 20 mg, between 1 mg and 19 mg, between 1 mg and 18 mg, between 1 mg and 17 mg, between 1 mg and 16 mg, between 1 mg and 15 mg, between 1 mg and 14 mg, between 1 mg and 13 mg, between 1 mg and 12 mg, between 1 mg and 11 mg, between 1 mg and 10 mg, between 1 mg and 9 mg, between 1 mg and 8 mg, between 1 mg and 7 mg, between 1 mg and 6 mg, between 1 mg and 5 mg, between 1 mg and 4 mg, between 1 mg and 2 mg, between 1 mg and 1.5 mg, between 1 mg and 3 mg, between 3 mg and 5 mg, between 5 mg and 7 mg, between 7 mg and 9 mg, between 9 mg and 14 mg, between 15 mg and 17 mg, between 18 mg and 31 mg, between 31 mg and 33 mg, between 0.5 mg and 2 mg, between 2 mg and 4 mg, between 11 mg and 13 mg, between 23 mg and 25 mg, between 2 mg and 31 mg, between 2 mg and 30 mg, between 2 mg and 29 mg, between 2 mg and 28 mg, between 2 mg and 27 mg, between 2 mg and 26 mg, between 2 mg and 25 mg, between 2 mg and 24 mg, between 2 mg and 23 mg, between 2 mg and 22 mg, between 2 mg and 21 mg, between 2 mg and 20 mg, between 2 mg and 19 mg, between 2 mg and 18 mg, between 2 mg and 17 mg, between 2 mg and 16 mg, between 2 mg and 15 mg, between 2 mg and 14 mg, between 2 mg and 13 mg, between 2 mg and 12 mg, between 2 mg and 11 mg, between 2 mg and 10 mg, between 2 mg and 9 mg, between 2 mg and 8 mg, between 2 mg and 7 mg, between 2 mg and 6 mg, between 2 mg and 5 mg, between 2 mg and 3 mg.

In some embodiments, administration of the SNA is at a dose (e.g., therapeutic dose or fixed dose) of or about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 30 mg, 13.5 mg, 14 mg, 14.5 mg, 15 mg, 15.5 mg, 16 mg, 16.5 mg, 17 mg, 70.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, administration of the SNA is at a dose (e.g., therapeutic dose or fixed dose) of at least or at least about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, administration of the SNA is at a dose (e.g., therapeutic dose or fixed dose) greater than or greater than about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 30 mg, 13.5 mg, 40 mg, 14.5 mg, 50 mg, 15.5 mg, 60 mg, 16.5 mg, 70 mg, 70.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, the method for treating a disease (e.g., cancer) comprises administration of an SNA and an agonist of a TNFRSF member to a subject; an immune checkpoint inhibitor and an agonist of a TNFRSF member to a subject; an SNA, an immune checkpoint inhibitor, and an agonist of a TNFRSF member to a subject, wherein the SNA comprises a core having an oligonucleotide shell comprised of CpG oligonucleotides positioned on the exterior of the core, wherein the SNA is administered to the subject at a dose (e.g., fixed dose) of between about 16 mg to about 32 mg to one solid tumor or tumor lesion or at a dose (e.g., fixed dose) of between about 16 mg to about 32 mg divided among two or more solid tumors or tumor lesions in the subject, wherein the SNA is administered within 24 hours of administration of the agonist of a TNFRSF member and/or within 24 hours of administration of the immune checkpoint inhibitor, to treat the disease (e.g., cancer) in the subject.

In some embodiments, administration of the SNA is at a dose of or about 1 mg. In some embodiments administration of the SNA is at a dose of or about 2 mg. In some embodiments administration of the SNA is at a dose of or about 3 mg. In some embodiments, administration of the SNA is at a dose of or about 4 mg. In some embodiments administration of the SNA is at a dose of or about 6 mg. In some embodiments, administration of the SNA is at a dose of or about 8 mg. In some embodiments, administration of the SNA is at a dose of or about 12 mg. In some embodiments, administration of the SNA is at a dose of or about 16 mg. In some embodiments, administration of the SNA is at a dose of or about 24 mg. In some embodiments administration of the SNA is at a dose of or about 32 mg.

In some embodiments, administration of the immune checkpoint inhibitor is at a dose of or about 200 mg. In some embodiments, administration of the immune checkpoint inhibitor is at a dose of or about 350 mg. In some embodiments, administration of the immune checkpoint inhibitor is at a dose of or about 375 mg. In some embodiments, administration of the immune checkpoint inhibitor is at a dose of or about 500 mg. In some embodiments, administration of the immune checkpoint inhibitor is at a dose of or about 750 mg. In some embodiments, administration of the immune checkpoint inhibitor is at a dose of or about 240 mg. In some embodiments, administration of the immune checkpoint inhibitor is at a dose of or about 480 mg. In some embodiments, administration of the immune checkpoint inhibitor is at a dose of or about 840 mg. In some embodiments, administration of the immune checkpoint inhibitor is at a dose of or about 1200 mg. In some embodiments, administration of the immune checkpoint inhibitor is at a dose of or about 1680 mg. In some embodiments, administration of the immune checkpoint inhibitor is at a dose of or about 1 mg/kg. In some embodiments, administration of the immune checkpoint inhibitor is at a dose of or about 3 mg/kg. In some embodiments, administration of the immune checkpoint inhibitor is at a dose of or about 10 mg/kg.

In some embodiments, administration of an agonist of a TNFRSF member is at a dose of or about 7 mg. In some embodiments, administration of an agonist of a TNFRSF member is at a dose of or about 20 mg. In some embodiments, administration of an agonist of a TNFRSF member is at a dose of or about 70 mg. In some embodiments, administration of an agonist of a TNFRSF member is at a dose of or about 200 mg. In some embodiments, administration of an agonist of a TNFRSF member is at a dose of or about 350 mg. In some embodiments, administration of an agonist of a TNFRSF member is at a dose of or about 700 mg. In some embodiments, administration of an agonist of a TNFRSF member is at a dose of or about 1400 mg.

In some embodiments, the method for treating a disease disclosed herein (e.g., cancer) comprises administering to a subject an agonist of a TNFRSF member; an agonist of a TNFRSF member and an SNA; or an agonist of a TNFRSF member, an SNA and an immune checkpoint inhibitor. In a non-limiting embodiment, the method for treating a disease disclosed herein (e.g., cancer) comprises administering to a subject an agonist of a TNFRSF member, an SNA and an immune checkpoint inhibitor in the combinations of doses disclosed below in Table 4. In some embodiments, the method for treating a disease (e.g., cancer) disclosed herein comprises administering to a subject two or three of a TNFRSF member, an SNA and an immune checkpoint inhibitor according to the combinations shown in Table 4. In some embodiments, combination doses (e.g., Combination Nos. 1-350) may include administration of two or three of the listed compounds (e.g., a dose of SNA and a dose of a TNFRSF member agonist; a dose of immune checkpoint inhibitor and a dose of a TNFRSF member agonist; a dose of SNA and a dose of immune checkpoint inhibitor; or a dose of SNA, a dose of immune checkpoint inhibitor and a dose of a TNFRSF member agonist).

TABLE 4
Non-limiting examples of doses of SNA, immune checkpoint
inhibitor and agonist of TNFRSF member in combinations
for treatment of a disease disclosed herein.

| Combination | | | Dose of Immune | Dose of TNFRSF | |
|---|---|---|---|---|---|
| No. | Dose of SNA | | Checkpoint Inhibitor | Member Agonist | |
| 1 | 1 | mg | 200 mg | 7 | mg |
| 2 | 1 | mg | 200 mg | 20 | mg |
| 3 | 1 | mg | 200 mg | 70 | mg |
| 4 | 1 | mg | 200 mg | 200 | mg |
| 5 | 1 | mg | 200 mg | 350 | mg |
| 6 | 1 | mg | 200 mg | 700 | mg |
| 7 | 1 | mg | 200 mg | 1400 | mg |
| 8 | 1 | mg | 350 mg | 7 | mg |
| 9 | 1 | mg | 350 mg | 20 | mg |
| 10 | 1 | mg | 350 mg | 70 | mg |
| 11 | 1 | mg | 350 mg | 200 | mg |
| 12 | 1 | mg | 350 mg | 350 | mg |
| 13 | 1 | mg | 350 mg | 700 | mg |
| 14 | 1 | mg | 350 mg | 1400 | mg |
| 15 | 1 | mg | 375 mg | 7 | mg |
| 16 | 1 | mg | 375 mg | 20 | mg |
| 17 | 1 | mg | 375 mg | 70 | mg |
| 18 | 1 | mg | 375 mg | 200 | mg |
| 19 | 1 | mg | 375 mg | 350 | mg |
| 20 | 1 | mg | 375 mg | 700 | mg |
| 21 | 1 | mg | 375 mg | 1400 | mg |
| 22 | 1 | mg | 500 mg | 7 | mg |
| 23 | 1 | mg | 500 mg | 20 | mg |
| 24 | 1 | mg | 500 mg | 70 | mg |
| 25 | 1 | mg | 500 mg | 200 | mg |
| 26 | 1 | mg | 500 mg | 350 | mg |
| 27 | 1 | mg | 500 mg | 700 | mg |
| 28 | 1 | mg | 500 mg | 1400 | mg |
| 29 | 1 | mg | 750 mg | 7 | mg |
| 30 | 1 | mg | 750 mg | 20 | mg |
| 31 | 1 | mg | 750 mg | 70 | mg |
| 32 | 1 | mg | 750 mg | 200 | mg |
| 33 | 1 | mg | 750 mg | 350 | mg |
| 34 | 1 | mg | 750 mg | 700 | mg |
| 35 | 1 | mg | 750 mg | 1400 | mg |
| 36 | 2 | mg | 200 mg | 7 | mg |
| 37 | 2 | mg | 200 mg | 20 | mg |
| 38 | 2 | mg | 200 mg | 70 | mg |
| 39 | 2 | mg | 200 mg | 200 | mg |
| 40 | 2 | mg | 200 mg | 350 | mg |
| 41 | 2 | mg | 200 mg | 700 | mg |
| 42 | 2 | mg | 200 mg | 1400 | mg |
| 43 | 2 | mg | 350 mg | 7 | mg |
| 44 | 2 | mg | 350 mg | 20 | mg |
| 45 | 2 | mg | 350 mg | 70 | mg |
| 46 | 2 | mg | 350 mg | 200 | mg |
| 47 | 2 | mg | 350 mg | 350 | mg |
| 48 | 2 | mg | 350 mg | 700 | mg |
| 49 | 2 | mg | 350 mg | 1400 | mg |
| 50 | 2 | mg | 375 mg | 7 | mg |
| 51 | 2 | mg | 375 mg | 20 | mg |
| 52 | 2 | mg | 375 mg | 70 | mg |
| 53 | 2 | mg | 375 mg | 200 | mg |
| 54 | 2 | mg | 375 mg | 350 | mg |
| 55 | 2 | mg | 375 mg | 700 | mg |
| 56 | 2 | mg | 375 mg | 1400 | mg |
| 57 | 2 | mg | 500 mg | 7 | mg |
| 58 | 2 | mg | 500 mg | 20 | mg |
| 59 | 2 | mg | 500 mg | 70 | mg |
| 60 | 2 | mg | 500 mg | 200 | mg |
| 61 | 2 | mg | 500 mg | 350 | mg |
| 62 | 2 | mg | 500 mg | 700 | mg |
| 63 | 2 | mg | 500 mg | 1400 | mg |
| 64 | 2 | mg | 750 mg | 7 | mg |
| 65 | 2 | mg | 750 mg | 20 | mg |
| 66 | 2 | mg | 750 mg | 70 | mg |
| 67 | 2 | mg | 750 mg | 200 | mg |
| 68 | 2 | mg | 750 mg | 350 | mg |
| 69 | 2 | mg | 750 mg | 700 | mg |
| 70 | 2 | mg | 750 mg | 1400 | mg |
| 71 | 3 | mg | 200 mg | 7 | mg |
| 72 | 3 | mg | 200 mg | 20 | mg |
| 73 | 3 | mg | 200 mg | 70 | mg |
| 74 | 3 | mg | 200 mg | 200 | mg |
| 75 | 3 | mg | 200 mg | 350 | mg |
| 76 | 3 | mg | 200 mg | 700 | mg |
| 77 | 3 | mg | 200 mg | 1400 | mg |
| 78 | 3 | mg | 350 mg | 7 | mg |
| 79 | 3 | mg | 350 mg | 20 | mg |
| 80 | 3 | mg | 350 mg | 70 | mg |
| 81 | 3 | mg | 350 mg | 200 | mg |
| 82 | 3 | mg | 350 mg | 350 | mg |
| 83 | 3 | mg | 350 mg | 700 | mg |
| 84 | 3 | mg | 350 mg | 1400 | mg |
| 85 | 3 | mg | 375 mg | 7 | mg |
| 86 | 3 | mg | 375 mg | 20 | mg |
| 87 | 3 | mg | 375 mg | 70 | mg |
| 88 | 3 | mg | 375 mg | 200 | mg |
| 89 | 3 | mg | 375 mg | 350 | mg |
| 90 | 3 | mg | 375 mg | 700 | mg |
| 91 | 3 | mg | 375 mg | 1400 | mg |
| 92 | 3 | mg | 500 mg | 7 | mg |
| 93 | 3 | mg | 500 mg | 20 | mg |
| 94 | 3 | mg | 500 mg | 70 | mg |
| 95 | 3 | mg | 500 mg | 200 | mg |
| 96 | 3 | mg | 500 mg | 350 | mg |
| 97 | 3 | mg | 500 mg | 700 | mg |
| 98 | 3 | mg | 500 mg | 1400 | mg |
| 99 | 3 | mg | 750 mg | 7 | mg |
| 100 | 3 | mg | 750 mg | 20 | mg |
| 101 | 3 | mg | 750 mg | 70 | mg |
| 102 | 3 | mg | 750 mg | 200 | mg |
| 103 | 3 | mg | 750 mg | 350 | mg |
| 104 | 3 | mg | 750 mg | 700 | mg |
| 105 | 3 | mg | 750 mg | 1400 | mg |
| 106 | 4 | mg | 200 mg | 7 | mg |
| 107 | 4 | mg | 200 mg | 20 | mg |
| 108 | 4 | mg | 200 mg | 70 | mg |
| 109 | 4 | mg | 200 mg | 200 | mg |
| 110 | 4 | mg | 200 mg | 350 | mg |
| 111 | 4 | mg | 200 mg | 700 | mg |
| 112 | 4 | mg | 200 mg | 1400 | mg |
| 113 | 4 | mg | 350 mg | 7 | mg |
| 114 | 4 | mg | 350 mg | 20 | mg |
| 115 | 4 | mg | 350 mg | 70 | mg |
| 116 | 4 | mg | 350 mg | 200 | mg |
| 117 | 4 | mg | 350 mg | 350 | mg |
| 118 | 4 | mg | 350 mg | 700 | mg |
| 119 | 4 | mg | 350 mg | 1400 | mg |
| 120 | 4 | mg | 375 mg | 7 | mg |
| 121 | 4 | mg | 375 mg | 20 | mg |
| 122 | 4 | mg | 375 mg | 70 | mg |
| 123 | 4 | mg | 375 mg | 200 | mg |
| 124 | 4 | mg | 375 mg | 350 | mg |
| 125 | 4 | mg | 375 mg | 700 | mg |
| 126 | 4 | mg | 375 mg | 1400 | mg |
| 127 | 4 | mg | 500 mg | 7 | mg |
| 128 | 4 | mg | 500 mg | 20 | mg |
| 129 | 4 | mg | 500 mg | 70 | mg |
| 130 | 4 | mg | 500 mg | 200 | mg |
| 131 | 4 | mg | 500 mg | 350 | mg |
| 132 | 4 | mg | 500 mg | 700 | mg |
| 133 | 4 | mg | 500 mg | 1400 | mg |
| 134 | 4 | mg | 750 mg | 7 | mg |
| 135 | 4 | mg | 750 mg | 20 | mg |
| 136 | 4 | mg | 750 mg | 70 | mg |
| 137 | 4 | mg | 750 mg | 200 | mg |
| 138 | 4 | mg | 750 mg | 350 | mg |
| 139 | 4 | mg | 750 mg | 700 | mg |
| 140 | 4 | mg | 750 mg | 1400 | mg |
| 141 | 6 | mg | 200 mg | 7 | mg |
| 142 | 6 | mg | 200 mg | 20 | mg |
| 143 | 6 | mg | 200 mg | 70 | mg |
| 144 | 6 | mg | 200 mg | 200 | mg |
| 145 | 6 | mg | 200 mg | 350 | mg |
| 146 | 6 | mg | 200 mg | 700 | mg |
| 147 | 6 | mg | 200 mg | 1400 | mg |
| 148 | 6 | mg | 350 mg | 7 | mg |
| 149 | 6 | mg | 350 mg | 20 | mg |
| 150 | 6 | mg | 350 mg | 70 | mg |
| 151 | 6 | mg | 350 mg | 200 | mg |
| 152 | 6 | mg | 350 mg | 350 | mg |
| 153 | 6 | mg | 350 mg | 700 | mg |
| 154 | 6 | mg | 350 mg | 1400 | mg |
| 155 | 6 | mg | 375 mg | 7 | mg |
| 156 | 6 | mg | 375 mg | 20 | mg |
| 157 | 6 | mg | 375 mg | 70 | mg |
| 158 | 6 | mg | 375 mg | 200 | mg |
| 159 | 6 | mg | 375 mg | 350 | mg |
| 160 | 6 | mg | 375 mg | 700 | mg |
| 161 | 6 | mg | 375 mg | 1400 | mg |
| 162 | 6 | mg | 500 mg | 7 | mg |
| 163 | 6 | mg | 500 mg | 20 | mg |
| 164 | 6 | mg | 500 mg | 70 | mg |
| 165 | 6 | mg | 500 mg | 200 | mg |
| 166 | 6 | mg | 500 mg | 350 | mg |
| 167 | 6 | mg | 500 mg | 700 | mg |
| 168 | 6 | mg | 500 mg | 1400 | mg |
| 169 | 6 | mg | 750 mg | 7 | mg |
| 170 | 6 | mg | 750 mg | 20 | mg |
| 171 | 6 | mg | 750 mg | 70 | mg |
| 172 | 6 | mg | 750 mg | 200 | mg |
| 173 | 6 | mg | 750 mg | 350 | mg |
| 174 | 6 | mg | 750 mg | 700 | mg |
| 175 | 6 | mg | 750 mg | 1400 | mg |
| 176 | 8 | mg | 200 mg | 7 | mg |
| 177 | 8 | mg | 200 mg | 20 | mg |
| 178 | 8 | mg | 200 mg | 70 | mg |
| 179 | 8 | mg | 200 mg | 200 | mg |
| 180 | 8 | mg | 200 mg | 350 | mg |
| 181 | 8 | mg | 200 mg | 700 | mg |
| 182 | 8 | mg | 200 mg | 1400 | mg |
| 183 | 8 | mg | 350 mg | 7 | mg |
| 184 | 8 | mg | 350 mg | 20 | mg |
| 185 | 8 | mg | 350 mg | 70 | mg |
| 186 | 8 | mg | 350 mg | 200 | mg |
| 187 | 8 | mg | 350 mg | 350 | mg |
| 188 | 8 | mg | 350 mg | 700 | mg |
| 189 | 8 | mg | 350 mg | 1400 | mg |
| 190 | 8 | mg | 375 mg | 7 | mg |
| 191 | 8 | mg | 375 mg | 20 | mg |
| 192 | 8 | mg | 375 mg | 70 | mg |
| 193 | 8 | mg | 375 mg | 200 | mg |
| 194 | 8 | mg | 375 mg | 350 | mg |
| 195 | 8 | mg | 375 mg | 700 | mg |
| 196 | 8 | mg | 375 mg | 1400 | mg |
| 197 | 8 | mg | 500 mg | 7 | mg |
| 198 | 8 | mg | 500 mg | 20 | mg |
| 199 | 8 | mg | 500 mg | 70 | mg |
| 200 | 8 | mg | 500 mg | 200 | mg |
| 201 | 8 | mg | 500 mg | 350 | mg |
| 202 | 8 | mg | 500 mg | 700 | mg |
| 203 | 8 | mg | 500 mg | 1400 | mg |
| 204 | 8 | mg | 750 mg | 7 | mg |
| 205 | 8 | mg | 750 mg | 20 | mg |
| 206 | 8 | mg | 750 mg | 70 | mg |
| 207 | 8 | mg | 750 mg | 200 | mg |
| 208 | 8 | mg | 750 mg | 350 | mg |
| 209 | 8 | mg | 750 mg | 700 | mg |
| 210 | 8 | mg | 750 mg | 1400 | mg |
| 211 | 12 | mg | 200 mg | 7 | mg |
| 212 | 12 | mg | 200 mg | 20 | mg |
| 213 | 12 | mg | 200 mg | 70 | mg |
| 214 | 12 | mg | 200 mg | 200 | mg |
| 215 | 12 | mg | 200 mg | 350 | mg |
| 216 | 12 | mg | 200 mg | 700 | mg |
| 217 | 12 | mg | 200 mg | 1400 | mg |
| 218 | 12 | mg | 350 mg | 7 | mg |
| 219 | 12 | mg | 350 mg | 20 | mg |
| 220 | 12 | mg | 350 mg | 70 | mg |
| 221 | 12 | mg | 350 mg | 200 | mg |
| 222 | 12 | mg | 350 mg | 350 | mg |
| 223 | 12 | mg | 350 mg | 700 | mg |
| 224 | 12 | mg | 350 mg | 1400 | mg |
| 225 | 12 | mg | 375 mg | 7 | mg |
| 226 | 12 | mg | 375 mg | 20 | mg |
| 227 | 12 | mg | 375 mg | 70 | mg |
| 228 | 12 | mg | 375 mg | 200 | mg |
| 229 | 12 | mg | 375 mg | 350 | mg |
| 230 | 12 | mg | 375 mg | 700 | mg |
| 231 | 12 | mg | 375 mg | 1400 | mg |
| 232 | 12 | mg | 500 mg | 7 | mg |
| 233 | 12 | mg | 500 mg | 20 | mg |
| 234 | 12 | mg | 500 mg | 70 | mg |
| 235 | 12 | mg | 500 mg | 200 | mg |
| 236 | 12 | mg | 500 mg | 350 | mg |
| 237 | 12 | mg | 500 mg | 700 | mg |
| 238 | 12 | mg | 500 mg | 1400 | mg |
| 239 | 12 | mg | 750 mg | 7 | mg |
| 240 | 12 | mg | 750 mg | 20 | mg |
| 241 | 12 | mg | 750 mg | 70 | mg |
| 242 | 12 | mg | 750 mg | 200 | mg |
| 243 | 12 | mg | 750 mg | 350 | mg |
| 244 | 12 | mg | 750 mg | 700 | mg |
| 245 | 12 | mg | 750 mg | 1400 | mg |
| 246 | 16 | mg | 200 mg | 7 | mg |
| 247 | 16 | mg | 200 mg | 20 | mg |
| 248 | 16 | mg | 200 mg | 70 | mg |
| 249 | 16 | mg | 200 mg | 200 | mg |
| 250 | 16 | mg | 200 mg | 350 | mg |
| 251 | 16 | mg | 200 mg | 700 | mg |
| 252 | 16 | mg | 200 mg | 1400 | mg |
| 253 | 16 | mg | 350 mg | 7 | mg |
| 254 | 16 | mg | 350 mg | 20 | mg |
| 255 | 16 | mg | 350 mg | 70 | mg |
| 256 | 16 | mg | 350 mg | 200 | mg |
| 257 | 16 | mg | 350 mg | 350 | mg |
| 258 | 16 | mg | 350 mg | 700 | mg |
| 259 | 16 | mg | 350 mg | 1400 | mg |
| 260 | 16 | mg | 375 mg | 7 | mg |
| 261 | 16 | mg | 375 mg | 20 | mg |
| 262 | 16 | mg | 375 mg | 70 | mg |
| 263 | 16 | mg | 375 mg | 200 | mg |
| 264 | 16 | mg | 375 mg | 350 | mg |
| 265 | 16 | mg | 375 mg | 700 | mg |
| 266 | 16 | mg | 375 mg | 1400 | mg |
| 267 | 16 | mg | 500 mg | 7 | mg |
| 268 | 16 | mg | 500 mg | 20 | mg |
| 269 | 16 | mg | 500 mg | 70 | mg |
| 270 | 16 | mg | 500 mg | 200 | mg |
| 271 | 16 | mg | 500 mg | 350 | mg |
| 272 | 16 | mg | 500 mg | 700 | mg |
| 273 | 16 | mg | 500 mg | 1400 | mg |
| 274 | 16 | mg | 750 mg | 7 | mg |
| 275 | 16 | mg | 750 mg | 20 | mg |
| 276 | 16 | mg | 750 mg | 70 | mg |
| 277 | 16 | mg | 750 mg | 200 | mg |
| 278 | 16 | mg | 750 mg | 350 | mg |
| 279 | 16 | mg | 750 mg | 700 | mg |
| 280 | 16 | mg | 750 mg | 1400 | mg |
| 281 | 24 | mg | 200 mg | 7 | mg |
| 282 | 24 | mg | 200 mg | 20 | mg |
| 283 | 24 | mg | 200 mg | 70 | mg |
| 284 | 24 | mg | 200 mg | 200 | mg |
| 285 | 24 | mg | 200 mg | 350 | mg |
| 286 | 24 | mg | 200 mg | 700 | mg |
| 287 | 24 | mg | 200 mg | 1400 | mg |
| 288 | 24 | mg | 350 mg | 7 | mg |
| 289 | 24 | mg | 350 mg | 20 | mg |
| 290 | 24 | mg | 350 mg | 70 | mg |
| 291 | 24 | mg | 350 mg | 200 | mg |
| 292 | 24 | mg | 350 mg | 350 | mg |
| 293 | 24 | mg | 350 mg | 700 | mg |
| 294 | 24 | mg | 350 mg | 1400 | mg |
| 295 | 24 | mg | 375 mg | 7 | mg |
| 296 | 24 | mg | 375 mg | 20 | mg |
| 297 | 24 | mg | 375 mg | 70 | mg |
| 298 | 24 | mg | 375 mg | 200 | mg |
| 299 | 24 | mg | 375 mg | 350 | mg |
| 300 | 24 | mg | 375 mg | 700 | mg |
| 301 | 24 | mg | 375 mg | 1400 | mg |
| 302 | 24 | mg | 500 mg | 7 | mg |
| 303 | 24 | mg | 500 mg | 20 | mg |
| 304 | 24 | mg | 500 mg | 70 | mg |
| 305 | 24 | mg | 500 mg | 200 | mg |
| 306 | 24 | mg | 500 mg | 350 | mg |
| 307 | 24 | mg | 500 mg | 700 | mg |
| 308 | 24 | mg | 500 mg | 1400 | mg |
| 309 | 24 | mg | 750 mg | 7 | mg |
| 310 | 24 | mg | 750 mg | 20 | mg |
| 311 | 24 | mg | 750 mg | 70 | mg |
| 312 | 24 | mg | 750 mg | 200 | mg |
| 313 | 24 | mg | 750 mg | 350 | mg |
| 314 | 24 | mg | 750 mg | 700 | mg |
| 315 | 24 | mg | 750 mg | 1400 | mg |
| 316 | 32 | mg | 200 mg | 7 | mg |
| 317 | 32 | mg | 200 mg | 20 | mg |
| 318 | 32 | mg | 200 mg | 70 | mg |
| 319 | 32 | mg | 200 mg | 200 | mg |
| 320 | 32 | mg | 200 mg | 350 | mg |
| 321 | 32 | mg | 200 mg | 700 | mg |
| 322 | 32 | mg | 200 mg | 1400 | mg |
| 323 | 32 | mg | 350 mg | 7 | mg |
| 324 | 32 | mg | 350 mg | 20 | mg |
| 325 | 32 | mg | 350 mg | 70 | mg |
| 326 | 32 | mg | 350 mg | 200 | mg |
| 327 | 32 | mg | 350 mg | 350 | mg |
| 328 | 32 | mg | 350 mg | 700 | mg |
| 329 | 32 | mg | 350 mg | 1400 | mg |
| 330 | 32 | mg | 375 mg | 7 | mg |
| 331 | 32 | mg | 375 mg | 20 | mg |
| 332 | 32 | mg | 375 mg | 70 | mg |
| 333 | 32 | mg | 375 mg | 200 | mg |
| 334 | 32 | mg | 375 mg | 350 | mg |
| 335 | 32 | mg | 375 mg | 700 | mg |
| 336 | 32 | mg | 375 mg | 1400 | mg |
| 337 | 32 | mg | 500 mg | 7 | mg |
| 338 | 32 | mg | 500 mg | 20 | mg |
| 339 | 32 | mg | 500 mg | 70 | mg |
| 340 | 32 | mg | 500 mg | 200 | mg |
| 341 | 32 | mg | 500 mg | 350 | mg |
| 342 | 32 | mg | 500 mg | 700 | mg |
| 343 | 32 | mg | 500 mg | 1400 | mg |
| 344 | 32 | mg | 750 mg | 7 | mg |
| 345 | 32 | mg | 750 mg | 20 | mg |
| 346 | 32 | mg | 750 mg | 70 | mg |
| 347 | 32 | mg | 750 mg | 200 | mg |
| 348 | 32 | mg | 750 mg | 350 | mg |
| 349 | 32 | mg | 750 mg | 700 | mg |
| 350 | 32 | mg | 750 mg | 1400 | mg |

As disclosed herein, the terms “solid tumor”, “tumor lesion”, “lesion” or “target lesion” are used interchangeably. In some embodiments, a solid tumor, tumor lesion, lesion or target lesion is an accessible solid tumor, accessible tumor lesion, accessible lesion, or an accessible target lesion. In some embodiments, the total dose of an SNA disclosed herein and/or the total volume of an SNA disclosed herein is administered to one solid tumor or tumor lesion in a subject in methods for treating cancer (e.g., BCC, such as locally advanced BCC, metastatic BCC, and/or superficial lesions in BCC, melanoma or liver cancer, such as HCC). In some embodiments, the subject has more than one solid tumor or tumor lesion. If the subject has more than one solid tumor or tumor lesion, the total dose of an SNA disclosed herein and/or the total volume of an SNA disclosed herein is divided equally among the solid tumors or tumor lesions in the subject. In some embodiments, the total dose of SNA and/or total volume of SNA administered to each solid tumor or tumor lesion in the subject may depend on various factors, such as on the number of injectable and/or accessible solid tumors or tumor lesions in the subject, the size of the smallest injectable and/or accessible solid tumor or tumor lesion in the subject, the concentration of SNA in a solution or formulation, and/or a minimum concentration for which syringe stability data are available.

For instance, in a non-limiting example, if an SNA disclosed herein is available in a solution or formulation at a concentration of 20 mg/mL, the subject is to receive a total dose of SNA of 4 mg and the subject has four solid tumors or tumor lesions, then each solid tumor or tumor lesion is to be injected with 0.050 mL of the SNA solution or formulation (at the concentration of 20 mg/mL). In a further non-limiting example, if the subject is to receive a total dose of the SNA of 4 mg and the subject has three solid tumors or tumor lesions, then each solid tumor or tumor lesion is to be injected with 0.067 mL of SNA solution or formulation (at the concentration of 20 mg/mL). In these non-limiting examples and for clarity, the concentration 20 mg/mL refers to the 20 mg of CpG oligonucleotides per mL of solution or formulation. In some embodiments, less than 4 mL of an SNA solution is injected into each tumor lesion. In some embodiments, less than 3 mL of an SNA solution is injected into each tumor lesion. In some embodiments, less than 2 mL of an SNA solution is injected into each tumor lesion. In some embodiments, less than 1 mL of an SNA solution is injected into each tumor lesion. In some embodiments, less than 4 mL of an SNA solution is injected into the sum of all tumor lesions in a subject. In some embodiments, less than 3 mL of an SNA solution is injected into the sum of all tumor lesions in a subject. In some embodiments, less than 2 mL of an SNA solution is injected into the sum of all tumor lesions in a subject. In some embodiments, less than 1 mL of an SNA solution is injected into the sum of all tumor lesions in a subject.

Thus, the total volume of SNA solution or formulation for a subject and the volume of SNA for each solid tumor or tumor lesion in the subject depends on the concentration of SNA solution or formulation, the dose of SNA for the subject and the number of injectable and/or accessible solid tumors or tumor lesions in the subject.

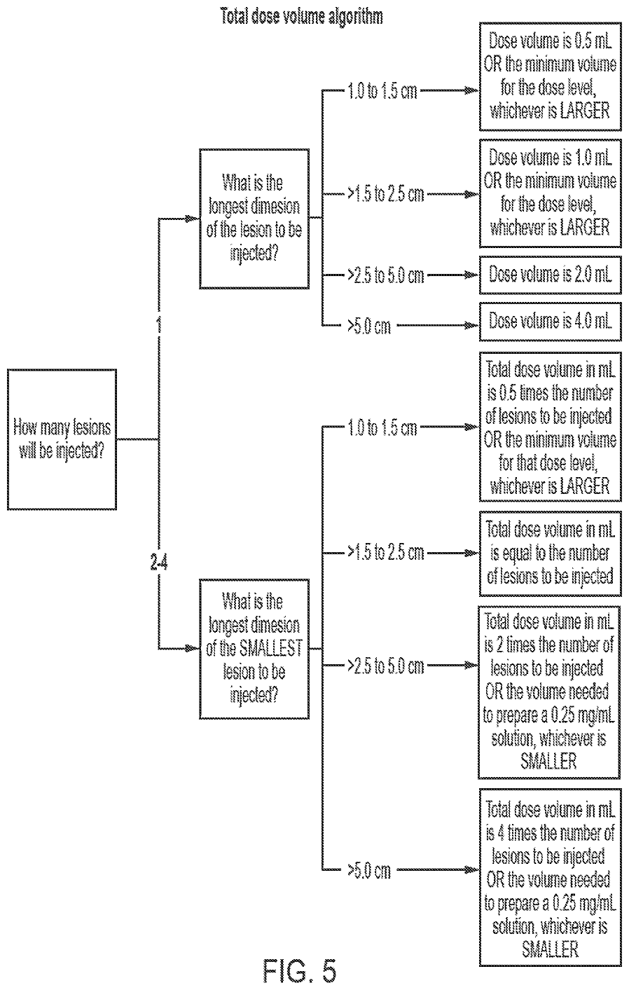

In some embodiments, the injectable and/or accessible solid tumors or tumor lesions are of different sizes. If the solid tumors or tumor lesions are of different sizes, the volume of SNA solution or formulation for each solid tumor or tumor lesion in the subject will depend on the longest dimension of the smallest lesion to be injected as described, for instance as a non-limiting example, in FIG. 5. In some embodiments, the total dose of SNA and/or the total volume of SNA is delivered or administered to one accessible tumor lesion or solid tumor, or divided among two accessible tumor lesions or solid tumors, three accessible tumor lesions or solid tumors, four accessible tumor lesions or solid tumors, five accessible tumor lesions or solid tumors, six accessible tumor lesions or solid tumors, seven accessible tumor lesions or solid tumors, eight accessible tumor lesions or solid tumors, nine accessible tumor lesions or solid tumors, or ten or more than 10 accessible tumor lesions or solid tumors. In some embodiments, the total dose of SNA and/or the total volume of SNA is delivered or administered into no more than four accessible tumor lesions or solid tumors. In some embodiments, the SNA is administered intratumorally (IT), cutaneously, subcutaneously, intravenously, by internal implantation, or into a lymph node.

In some embodiments, the total dose of SNA and/or the total volume of SNA is not divided equally among the solid tumors or tumor lesions in a subject that has more than one solid tumor or tumor lesion. In some embodiments, the SNA dose administered to one solid tumor or tumor lesion is greater than the SNA dose that would be administered to one solid tumor or tumor lesion if the SNA dose was divided equally among all the accessible and/or injectable solid tumors or tumor lesions in the subject. In some embodiments, administration of an SNA or administration of an SNA in combination with a checkpoint inhibitor to a subject with cancer (e.g., BCC, such as locally advanced BCC, metastatic BCC, and/or superficial lesions in BCC, melanoma or liver cancer, such as HCC) results in reduced diameter of one or more solid tumors or tumor lesions or complete disappearance of one or more solid tumors or tumor lesions which have not directly received a dose of the SNA (e.g., the SNA has not been injected into the solid tumor or tumor lesion).

For instance, in a non-limiting example, a subject having three solid tumors or tumor lesions is administered a total SNA dose of 4 mg, wherein one solid tumor or tumor lesion receives an SNA dose of 2 mg and each of the two remaining solid tumors or tumor lesions receive an SNA dose of 1 mg, for a total SNA dose of 4 mg administered to the subject. In some embodiments, the SNA dose administered to one solid tumor or tumor lesion is less than the SNA dose that would be administered to one solid tumor or tumor lesion if the SNA dose was divided equally among all the accessible and/or injectable solid tumors or tumor lesions in the subject. For instance, in a non-limiting example, a subject having three solid tumors or tumor lesions is administered a total SNA dose of 4 mg, wherein one solid tumor or tumor lesion receives an SNA dose of 0.5 mg and each of the two remaining solid tumors or tumor lesions receive an SNA dose of 1.75 mg, for a total SNA dose of 4 mg administered to the subject.

In some embodiments, administration of an SNA, an agonist of a TNFRSF member, an immune checkpoint inhibitor, or a combination of two or more of an SNA, an agonist of a TNFRSF member and an immune checkpoint inhibitor to a subject with cancer results in reduced diameter of one or more solid tumors or tumor lesions or complete disappearance of one or more solid tumors or tumor lesions which have not directly received a dose of a therapeutic (e.g., the SNA and/or the agonist of a TNFRSF member has not been injected into the solid tumor or tumor lesion).

In some embodiments, the term “multiple lesions” refers to two or more solid tumors, tumor lesions or target lesions. In some embodiments, the term multiple lesions refers to at least two, at least three, at least four, at least five, at least six, at least seven, at least eight, at least nine, at least 10, at least 11, or at least 12 solid tumors, tumor lesions or target lesions, or any range or combination thereof.

In some embodiments, a dose is a therapeutic dose. In some embodiments, a dose is a fixed dose. In some embodiments, the SNA is administered at a dose (e.g., therapeutic dose or fixed dose) that results in a measurable concentration of SNA in the plasma of a subject. In some embodiments, the SNA is administered at a dose (e.g., therapeutic dose or fixed dose) that results in a measurable concentration of SNA in the plasma of a subject of between 0.1 ng/mL and 1000 ng/mL. In some embodiments, the SNA is administered at a dose (e.g., therapeutic dose or fixed dose) that results in a measurable concentration of SNA in the plasma of a subject of between 0.2 ng/mL and 1000 ng/mL, between 1 ng/mL and 1000 ng/mL, between 2 ng/mL and 1000 ng/mL, between 3 ng/mL and 1000 ng/mL, between 4 ng/mL and 1000 ng/mL, between 5 ng/mL and 1000 ng/mL, between 6 ng/mL and 1000 ng/mL, between 7 ng/mL and 1000 ng/mL, between 8 ng/mL and 1000 ng/mL, between 9 ng/mL and 1000 ng/mL, or between 10 ng/mL and 1000 ng/mL.

In some embodiments, the SNA is administered at a dose (e.g., therapeutic dose or fixed dose) that results in concentrations of SNA in the plasma of a subject of between 1 ng/mL and 200 ng/mL, between 1 ng/mL and 100 ng/mL, between 1 ng/mL and 90 ng/mL, between 1 ng/mL and 80 ng/mL, between 1 ng/mL and 70 ng/mL, between 1 ng/mL and 60 ng/mL, between 1 ng/mL and 50 ng/mL, between 1 ng/mL and 40 ng/mL, between 1 ng/mL and 30 ng/mL, between 1 ng/mL and 20 ng/mL, between 1 ng/mL and 10 ng/mL, between 1 ng/mL and 9 ng/mL, between 1 ng/mL and 8 ng/mL, between 1 ng/mL and 7 ng/mL, between 1 ng/mL and 6 ng/mL, between 1 ng/mL and 5 ng/mL, between 1 ng/mL and 4 ng/mL, between 1 ng/mL and 3 ng/mL, or between 1 ng/mL and 2 ng/mL.

In some embodiments, the immune checkpoint inhibitor is administered at a dose (e.g., therapeutic dose or fixed dose) of between 50 mg and 1000 mg, between 50 mg and 750 mg, between 50 mg and 500 mg, between 50 mg and 400 mg, between 50 mg and 350 mg, between 50 mg and 300 mg, between 50 mg and 290 mg, between 50 mg and 280 mg, between 50 mg and 270 mg, between 50 mg and 260 mg, between 50 mg and 250 mg, between 50 mg and 240 mg, between 50 mg and 230 mg, between 50 mg and 220 mg, between 50 mg and 210 mg, between 50 mg and 200 mg, between 60 mg and 200 mg, between 70 mg and 200 mg, between 80 mg and 200 mg, between 90 mg and 200 mg, between 100 mg and 200 mg, between 110 mg and 200 mg, between 120 mg and 200 mg, between 130 mg and 200 mg, between 140 mg and 200 mg, between 150 mg and 200 mg, between 160 mg and 200 mg, between 170 mg and 200 mg, between 180 mg and 200 mg, or between 190 mg and 200 mg, between 200 mg and 500 mg, between 200 mg and 450 mg, between 200 mg and 440 mg, between 200 mg and 430 mg, between 200 mg and 420 mg, between 200 mg and 410 mg, between 200 mg and 400 mg, between 200 mg and 390 mg, between 200 mg and 380 mg, 200 mg and 370 mg, between 200 mg and 360 mg, between 200 mg and 350 mg, between 210 mg and 350 mg, between 220 mg and 350 mg, between 230 mg and 350 mg, between 240 mg and 350 mg, between 250 mg and 350 mg, between 260 mg and 350 mg, between 270 mg and 350 mg, between 280 mg and 350 mg, between 290 mg and 350 mg, between 300 mg and 350 mg, between 310 mg and 350 mg, between 320 mg and 350 mg, between 330 mg and 350 mg, or between 340 mg and 350 mg.

In some embodiments, the immune checkpoint inhibitor is administered at a dose (e.g., therapeutic dose or fixed dose) of or about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, or 2000 mg, or any range or combination thereof. In some embodiments, the immune checkpoint inhibitor is pembrolizumab administered at a dose of between 180 mg and 220 mg, avelumab administered at a dose of between 700 mg and 900 mg, or cemiplimab administered at a dose of between 330 mg and 370 mg. In some embodiments, the immune checkpoint inhibitor is retifanlimab administered at a dose of between 150 and 850 mg.

In some embodiments, the immune checkpoint inhibitor is administered at a dose (e.g., therapeutic dose or fixed dose) of at least or at least about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, or 2000 mg, or any range or combination thereof.

In some embodiments, the immune checkpoint inhibitor is administered at a dose (e.g., therapeutic dose or fixed dose) greater than or greater than about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, or 2000 mg, or any range or combination thereof.

In some embodiments, the immune checkpoint inhibitor is administered at a dose (e.g., therapeutic dose or fixed dose) of or about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, or 50 mg/kg, or any range or combination thereof.

In some embodiments, the agonist of a TNFRSF member is administered at a dose (e.g., therapeutic dose or fixed dose) of between 5 mg and 1500 mg, between 7 and 1400 mg, between 20 and 1000 mg, between 50 mg and 1000 mg, between 50 mg and 750 mg, between 50 mg and 500 mg, between 50 mg and 400 mg, between 50 mg and 350 mg, between 50 mg and 300 mg, between 50 mg and 290 mg, between 50 mg and 280 mg, between 50 mg and 270 mg, between 50 mg and 260 mg, between 50 mg and 250 mg, between 50 mg and 240 mg, between 50 mg and 230 mg, between 50 mg and 220 mg, between 50 mg and 210 mg, between 50 mg and 200 mg, between 60 mg and 200 mg, between 70 mg and 200 mg, between 80 mg and 200 mg, between 90 mg and 200 mg, between 100 mg and 200 mg, between 110 mg and 200 mg, between 120 mg and 200 mg, between 130 mg and 200 mg, between 140 mg and 200 mg, between 150 mg and 200 mg, between 160 mg and 200 mg, between 170 mg and 200 mg, between 180 mg and 200 mg, or between 190 mg and 200 mg, between 200 mg and 500 mg, between 200 mg and 450 mg, between 200 mg and 440 mg, between 200 mg and 430 mg, between 200 mg and 420 mg, between 200 mg and 410 mg, between 200 mg and 400 mg, between 200 mg and 390 mg, between 200 mg and 380 mg, 200 mg and 370 mg, between 200 mg and 360 mg, between 200 mg and 350 mg, between 210 mg and 350 mg, between 220 mg and 350 mg, between 230 mg and 350 mg, between 240 mg and 350 mg, between 250 mg and 350 mg, between 260 mg and 350 mg, between 270 mg and 350 mg, between 280 mg and 350 mg, between 290 mg and 350 mg, between 300 mg and 350 mg, between 310 mg and 350 mg, between 320 mg and 350 mg, between 330 mg and 350 mg, or between 340 mg and 350 mg.

In some embodiments, administration of an agonist of a TNFRSF member is at a dose (e.g., therapeutic dose or fixed dose) of or about 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, or 2000 mg, or any range or combination thereof. In some embodiments, the agonist of a TNFRSF member is INCAGN1949 administered at a dose of between 7 mg and 1400 mg.

In some embodiments, administration of an agonist of a TNFRSF member is at a dose (e.g., therapeutic dose or fixed dose) of at least or at least about 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, or 2000 mg, or any range or combination thereof.

In some embodiments, administration of an agonist of a TNFRSF member is at a dose (e.g., therapeutic dose or fixed dose) greater than or greater than about 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, or 2000 mg, or any range or combination thereof.

In some embodiments, administration of an agonist of a TNFRSF member is at a dose (e.g., therapeutic dose or fixed dose) of or about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, or 50 mg/kg, or any range or combination thereof.

In some embodiments, administration of an SNA disclosed herein is at a dose (e.g., a therapeutic dose) disclosed herein in combination with an agonist of a TNFRSF member disclosed herein at a dose (e.g., a therapeutic dose) disclosed herein in methods for treating a disease (e.g., cancer). In some embodiments, an SNA disclosed herein is administered at a dose (e.g., a therapeutic dose) disclosed herein in combination with an agonist of a TNFRSF member disclosed herein at a dose (e.g., a therapeutic dose) disclosed herein and an immune checkpoint inhibitor disclosed herein at a dose (e.g., a therapeutic dose) disclosed herein in methods for treating cancer. For instance, a non-limiting example includes the administration of an SNA disclosed herein to a subject at a dose of 2 mg, the administration of an agonist of a TNFRSF member disclosed herein at a dose of 200 mg, and the administration of an immune checkpoint inhibitor disclosed herein at a dose of 200 mg. The combination of any of the dose amounts or dose ranges disclosed herein are also contemplated herein in methods for treating a disease (e.g., cancer) in a subject.

In some embodiments, a dose disclosed herein is considered a fixed dose or a discrete dose. In some embodiments, a dose disclosed herein can be adjusted to depend on body weight or made dependent on body weight. A non-limiting example includes a dose of 2 mg of SNA, as disclosed herein, that can also be administered as 2 mg/kg body weight, which depends on kg body weight, or a dose of 200 mg of an agonist of a TNFRSF member that can also be administered as 200 mg/kg body weight.

In some embodiments, the cancer is melanoma, mucosal melanoma, cutaneous melanoma, metastatic malignant melanoma, renal cancer, clear cell carcinoma, prostate cancer, hormone refractory prostate adenocarcinoma, breast cancer, colon cancer, lung cancer, non-small cell lung cancer, bone cancer, pancreatic cancer, pancreatic adenocarcinoma, skin cancer, cancer of the head or neck, cutaneous or intraocular malignant melanoma, uterine cancer, ovarian cancer, rectal cancer, stomach cancer, testicular cancer, thyroid cancer, anaplastic thyroid cancer, uterine cancer, carcinoma of the fallopian tubes, carcinoma of the endometrium, carcinoma of the cervix, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, biliary tract cancer, brain cancer, breast cancer, cervical cancer, choriocarcinoma, esophageal cancer, gastric cancer, an intraepithelial neoplasm, lymphoma, liver cancer, neuroblastoma, oral cancer, sarcomas, hairy cell leukemia, chronic myelogenous leukemia, cutaneous T-cell leukemia, multiple myeloma, renal cell carcinoma, lymphoma, bladder cancer, non-small cell lung cancer (NSCLC), glioblastoma multiforme, Merkel cell carcinoma, basal cell carcinoma (BCC, such as locally advanced BCC, metastatic BCC, and/or superficial BCC), cutaneous squamous cell carcinoma, melanoma or squamous cell carcinoma of the head and neck, environmentally induced cancers including those induced by asbestos, or any combinations thereof. In some embodiments, the cancer is not melanoma. In some embodiments, the cancer is non-melanoma skin cancer. In some embodiments, the cancer is not breast cancer. In some embodiments, the cancer is BCC. In some embodiments, the cancer is superficial BCC. In some embodiments, the cancer is locally advanced BCC, metastatic BCC, or both locally advanced and metastatic BCC. In some embodiments, the cancer involves superficial and locally advanced or metastatic BCC lesions.

In some embodiments, the cancer is a sarcoma. In some embodiments, the sarcoma is pleomorphic sarcoma, gastrointestinal stromal tumor (GIST), liposarcoma, leiomyosarcoma, synovial sarcoma, malignant peripheral nerve sheath tumor, rhabdomyosarcoma, angiosarcoma, fibrosarcoma, dermatofibrosarcoma protuberans, epithelioid sarcoma, myxoma, mesenchymoma, vascular sarcoma, neurilemmoma, bone sarcoma, osteosarcoma, Ewing's sarcoma, chondrosarcoma, Kaposi sarcoma, solitary fibrous tumor, chordoma, desmoid-type fibromatosis, fibroblastic sarcoma, giant cell tumor of the bone, gynecological sarcoma, soft tissue sarcoma, angioleiomyoma, leiomyoma, smooth muscle sarcoma, or fibrohistiocytic sarcoma.

In some embodiments, a cancer is classified according to the type of cells involved in the cancer, according to the cell type from which the cancer originated, and/or according to the organ/tissue in which the cancer developed or is advancing.

In some embodiments, the cancer is an anal cancer. In some embodiments, the anal cancer is anal carcinoma. In some embodiments, the anal cancer is squamous cell carcinoma. In some embodiments, the anal cancer is basal cell carcinoma (e.g., Bowen disease). In some embodiments, the anal cancer is cloacogenic carcinoma. In some embodiments, the anal cancer is an anal melanoma. In some embodiments, the anal cancer is an anal lymphoma.

In some embodiments, the cancer is a bladder cancer. In some embodiments, the bladder cancer is a bladder carcinoma. In some embodiments, the bladder cancer is transitional cell carcinoma, such as papillary carcinoma or flat carcinoma. In some embodiments, the bladder cancer is squamous cell carcinoma. In some embodiments, the bladder cancer is adenocarcinoma. In some embodiments, the bladder cancer is small cell carcinoma. In some embodiments, the bladder cancer is a sarcoma.

In some embodiments, the cancer is a bone cancer. In some embodiments, the bone cancer is a myeloma. In some embodiments, the bone cancer is multiple myeloma. In some embodiments, the bone cancer is a sarcoma, such as fibrosarcoma or undifferentiated pleomorphic sarcoma. In some embodiments, the bone cancer is osteosarcoma. In some embodiments, the bone cancer malignant fibrous histiocytoma. In some embodiments, the bone cancer is adamantinoma. In some embodiments, the bone cancer is chondrosarcoma, such as dedifferentiated chondrosarcoma, mesenchymal chondrosarcoma, or clear cell chondrosarcoma. In some embodiments, the bone cancer is chordoma. In some embodiments, the bone cancer is Ewing sarcoma of the bone. In some embodiments, the bone cancer is a lymphoma. In some embodiments, the bone cancer is a primary non-Hodgkin lymphoma of bone. In some embodiments, the bone cancer arises from giant cell tumor of bone.

In some embodiments, the cancer is a breast cancer. In some embodiments, the breast cancer is a carcinoma. In some embodiments, the breast cancer is ductal carcinoma in situ. In some embodiments, the breast cancer is invasive ductal carcinoma. In some embodiments, the breast cancer is triple-negative breast cancer. In some embodiments, the breast cancer is inflammatory breast cancer. In some embodiments, the breast cancer is tubular carcinoma of the breast. In some embodiments, the breast cancer is cribriform carcinoma of the breast. In some embodiments, the breast cancer is medullary carcinoma of the breast. In some embodiments, the breast cancer is mucinous carcinoma of the breast. In some embodiments, the breast cancer is papillary carcinoma of the breast. In some embodiments, the breast cancer is lobular carcinoma in situ. In some embodiments, the breast cancer is Paget's disease of the nipple. In some embodiments, the breast cancer arises from phyllodes tumors of the breast. In some embodiments, the breast cancer is a sarcoma. In some embodiments, the breast cancer is angiosarcoma.

In some embodiments, the cancer is a heart cancer. In some embodiments, the heart cancer is a sarcoma. In some embodiments, the heart cancer is angiosarcoma.

In some embodiments, the cancer is a cervical cancer. In some embodiments, the cervical cancer is a carcinoma. In some embodiments, the cervical cancer is squamous cell carcinoma. In some embodiments, the cervical cancer is adenocarcinoma. In some embodiments, the cervical cancer arises from infection caused by the human papillomavirus.

In some embodiments, the cancer is a colorectal cancer. In some embodiments, the colorectal cancer is a carcinoma. In some embodiments, the colorectal cancer is adenocarcinoma. In some embodiments, the colorectal cancer is mucinous adenocarcinoma. In some embodiments, the colorectal cancer is Signet ring cell adenocarcinoma. In some embodiments, the colorectal cancer is a lymphoma. In some embodiments, the colorectal cancer arises from gastrointestinal carcinoid tumors. In some embodiments, the colorectal cancer arises from gastrointestinal stromal tumors. In some embodiments, the colorectal cancer is leiomyosarcoma.

In some embodiments, the cancer is a uterine cancer. In some embodiments, the uterine cancer is a sarcoma. In some embodiments, the uterine cancer is leiomyosarcoma. In some embodiments, the uterine cancer is endometrial stromal sarcoma. In some embodiments, the uterine cancer is undifferentiated sarcoma. In some embodiments, the uterine cancer is an endometrial cancer. In some embodiments, the endometrial cancer is a carcinoma. In some embodiments, the endometrial cancer is adenocarcinoma. In some embodiments, the endometrial cancer is uterine carcinosarcoma. In some embodiments, the endometrial cancer is squamous cell carcinoma. In some embodiments, the endometrial cancer is small cell carcinoma. In some embodiments, the endometrial cancer is transitional carcinoma. In some embodiments, the endometrial cancer is serous carcinoma. In some embodiments, the endometrial cancer is clear-cell carcinoma. In some embodiments, the endometrial cancer is mucinous adenocarcinoma. In some embodiments, the endometrial cancer is undifferentiated carcinoma. In some embodiments, the endometrial cancer is dedifferentiated carcinoma. In some embodiments, the endometrial cancer is serous adenocarcinoma. In some embodiments, the endometrial cancer is adenoacanthoma. In some embodiments, the endometrial cancer is adenosquamous. In some embodiments, the endometrial cancer is secretory carcinoma. In some embodiments, the endometrial cancer is ciliated carcinoma. In some embodiments, the endometrial cancer is villoglandular adenocarcinoma.

In some embodiments, the cancer is an esophageal cancer. In some embodiments, the esophageal cancer is a carcinoma. In some embodiments, the esophageal cancer is adenocarcinoma. In some embodiments, the esophageal cancer is squamous cell carcinoma. In some embodiments, the esophageal cancer is small cell carcinoma.

In some embodiments, the cancer is an ocular cancer. In some embodiments, the ocular cancer is a melanoma. In some embodiments, the ocular cancer is eye melanoma. In some embodiments, the ocular cancer is uveal melanoma. In some embodiments, the ocular cancer is a carcinoma. In some embodiments, the ocular cancer is a squamous cell carcinoma. In some embodiments, the ocular cancer is basal cell carcinoma. In some embodiments, the ocular cancer is sebaceous carcinoma. In some embodiments, the ocular cancer is adenoid cystic carcinoma. In some embodiments, the ocular cancer is pleomorphic adenoma. In some embodiments, the ocular cancer is transitional cell carcinoma. In some embodiments, the ocular cancer is Merkel cell carcinoma. In some embodiments, the ocular cancer is a lymphoma. In some embodiments, the ocular cancer is mucosa-associated lymphoid tissue lymphoma. In some embodiments, the ocular cancer is orbital lymphoma. In some embodiments, the ocular cancer is lacrimal sac lymphoma. In some embodiments, the ocular cancer is a sarcoma. In some embodiments, the ocular cancer is orbital sarcoma. In some embodiments, the ocular cancer is retinoblastoma.

In some embodiments, the cancer is a fallopian tube cancer. In some embodiments, the fallopian tube cancer is a carcinoma. In some embodiments, the fallopian tube cancer is serous adenocarcinoma. In some embodiments, the fallopian tube cancer is papillary serous adenocarcinoma. In some embodiments, the fallopian tube cancer is endometroid adenocarcinoma. In some embodiments, the fallopian tube cancer is a sarcoma. In some embodiments, the fallopian tube cancer is leiomyosarcoma. In some embodiments, the fallopian tube cancer is transitional cell carcinoma.

In some embodiments, the cancer is a gallbladder cancer. In some embodiments, the gallbladder cancer is a carcinoma. In some embodiments, the gallbladder cancer is nonpapillary adenocarcinoma. In some embodiments, the gallbladder cancer is papillary adenocarcinoma. In some embodiments, the gallbladder cancer is mucinous adenocarcinoma. In some embodiments, the gallbladder cancer is adenosquamous carcinoma. In some embodiments, the gallbladder cancer is squamous cell carcinoma. In some embodiments, the gallbladder cancer is carcinosarcoma.

In some embodiments, the cancer is a cancer of the blood and bone marrow. In some embodiments, the cancer of the blood and bone marrow is leukemia. In some embodiments, the cancer of the blood and bone marrow is non-Hodgkin lymphoma. In some embodiments, the cancer of the blood and bone marrow is Hodgkin lymphoma. In some embodiments, the cancer of the blood and bone marrow is multiple myeloma.

In some embodiments, the cancer is a leukemia. In some embodiments, the leukemia is acute lymphocytic leukemia. In some embodiments, the leukemia is acute myeloid leukemia. In some embodiments, the leukemia is chronic lymphocytic leukemia. In some embodiments, the leukemia is chronic myeloid leukemia. In some embodiments, the leukemia is hairy cell leukemia. In some embodiments, the leukemia arises from myelodysplastic syndromes. In some embodiments, the leukemia is adult T-cell leukemia.

In some embodiments, the cancer is a non-Hodgkin lymphoma. In some embodiments, the non-Hodgkin lymphoma is diffuse large B-cell lymphoma. In some embodiments, the non-Hodgkin lymphoma is anaplastic large-cell lymphoma. In some embodiments, the non-Hodgkin lymphoma is Burkitt lymphoma. In some embodiments, the non-Hodgkin lymphoma is lymphoblastic lymphoma. In some embodiments, the non-Hodgkin lymphoma is mantle cell lymphoma. In some embodiments, the non-Hodgkin lymphoma is peripheral T-cell lymphoma. In some embodiments, the non-Hodgkin lymphoma is follicular lymphoma. In some embodiments, the non-Hodgkin lymphoma is cutaneous T-cell lymphoma. In some embodiments, the non-Hodgkin lymphoma is lymphoplasmacytic lymphoma. In some embodiments, the non-Hodgkin lymphoma is marginal zone B-cell lymphoma. In some embodiments, the non-Hodgkin lymphoma is mucosa-associated lymphoid tissue lymphoma. In some embodiments, the non-Hodgkin lymphoma is small-cell lymphocytic lymphoma. In some embodiments, the non-Hodgkin lymphoma is small lymphocytic lymphoma. In some embodiments, the non-Hodgkin lymphoma is precursor T-lymphoblastic lymphoma. In some embodiments, the non-Hodgkin lymphoma is adult T-cell lymphoma.

In some embodiments, the cancer is a Hodgkin lymphoma. In some embodiments, the Hodgkin lymphoma is nodular sclerosis. In some embodiments, the Hodgkin lymphoma is mixed cellularity. In some embodiments, the Hodgkin lymphoma is lymphocyte-rich. In some embodiments, the Hodgkin lymphoma is lymphocyte-depleted. In some embodiments, the Hodgkin lymphoma is nodular lymphocyte-predominant Hodgkin lymphoma.

In some embodiments, the cancer is a brain cancer. In some embodiments, the brain cancer arises from glioblastomas. In some embodiments, the brain cancer arises from ependymomas. In some embodiments, the brain cancer arises from oligodendrogliomas. In some embodiments, the brain cancer arises from astrocytomas. In some embodiments, the brain cancer arises from glioblastoma multiforme. In some embodiments, the brain cancer arises from meningiomas. In some embodiments, the brain cancer arises from pituitary carcinomas. In some embodiments, the brain cancer arises from craniopharyngiomas. In some embodiments, the brain cancer arises from germ cell tumors. In some embodiments, the brain cancer arises from pineal region tumors. In some embodiments, the brain cancer arises from medulloblastomas. In some embodiments, the brain cancer arises from primary central nervous system lymphomas.

In some embodiments, the cancer is an oral cancer. In some embodiments, the oral cancer is a carcinoma. In some embodiments, the oral cancer is squamous cell carcinoma. In some embodiments, the oral cancer is verrucous carcinoma. In some embodiments, the oral cancer is minor salivary gland carcinoma. In some embodiments, the oral cancer is adenoid cystic carcinoma. In some embodiments, the oral cancer is mucoepidermoid carcinoma. In some embodiments, the oral cancer is polymorphous low-grade adenocarcinoma. In some embodiments, the oral cancer is a lymphoma. In some embodiments, the oral cancer is a lip cancer. In some embodiments, the lip cancer is a carcinoma. In some embodiments, the lip cancer is squamous cell carcinoma. In some embodiments, the lip cancer is basal cell carcinoma. In some embodiments, the oral cancer is a mouth cancer. In some embodiments, the oral cancer is a tongue cancer.

In some embodiments, the cancer is a kidney cancer. In some embodiments, the kidney cancer is a carcinoma. In some embodiments, the kidney cancer is renal cell carcinoma. In some embodiments, the kidney cancer is clear cell renal carcinoma. In some embodiments, the kidney cancer is papillary renal cell carcinoma. In some embodiments, the kidney cancer is chromophobe renal cell carcinoma. In some embodiments, the kidney cancer is collecting duct renal cell carcinoma. In some embodiments, the kidney cancer is unclassified renal cell carcinoma. In some embodiments, the kidney cancer is transitional cell carcinoma. In some embodiments, the kidney cancer is a sarcoma. In some embodiments, the kidney cancer is renal sarcoma. In some embodiments, the kidney cancer arises from Wilms tumor. In some embodiments, the kidney cancer is a lymphoma.

In some embodiments, the cancer is a liver cancer. In some embodiments, the liver cancer is a carcinoma. In some embodiments, the liver cancer is HCC. In some embodiments, the liver cancer is fibrolamellar HCC. In some embodiments, the liver cancer is cholangiocarcinoma. In some embodiments, the liver cancer is intrahepatic cholangiocarcinoma. In some embodiments, the liver cancer is a sarcoma. In some embodiments, the liver cancer is angiosarcoma. In some embodiments, the liver cancer is hemangiosarcoma. In some embodiments, the liver cancer arises from hepatoblastomas.

In some embodiments, the liver cancer is associated with a chronic infection, such as infection with the hepatitis B virus or infection with the hepatitis C virus. In some embodiments, the liver cancer is associated with a chronic liver disease, like cirrhosis. In some embodiments, the liver cancer is associated with nonalcoholic fatty liver disease, such as non-alcoholic steatohepatitis. In some embodiments, the liver cancer is associated with inherited metabolic disease, such as hereditary hemochromatosis. In some embodiments, the liver cancer is associated with an autoimmune disease, like primary biliary cirrhosis. In some embodiments, the liver cancer is associated with heavy alcohol use, tobacco use, obesity, or type 2 diabetes. In some embodiments, the liver cancer is associated with rare diseases, like tyrosinemia, alpha-1 antitrypsin deficiency, porphyria cutanea tarda, primary biliary cholangitis, glycogen storage diseases, or Wilson disease. In some embodiments, the liver cancer is associated with exposure to aflatoxins, vinyl chloride, thorium dioxide, or anabolic steroids. In some embodiments, the liver cancer is associated with liver lesion. In some embodiments, the liver cancer is associated with metabolic biomarkers, such as α-Fetoprotein, des-γ-carboxyprothrombin, glypican-3, Golgi protein-73, and squamous cell carcinoma antigen. In some embodiments, the liver cancer is associated with chromosomal instability. In some embodiments, the chromosomal instability is characterized by amplification or gain of chromosomal segments or arms, such as at chromosome 1q and 8q. In some embodiments, the chromosomal instability is characterized by deletion or loss of chromosomal segments or arms, such as at chromosome 1p, 4q, 6q, 9p, 16p, 16q, or 17p. In some embodiments, the liver cancer is associated with global DNA hypomethylation. In some embodiments, the liver cancer is associated with mutations in the Wnt pathway, such as in CTNNB1, AXIN1, or APC. In some embodiments, the liver cancer is associated with mutations in cell cycle control, like TP53, RBI, CCNA2, CCNE1, PTEN, ARID1A, ARID2, RPS6KA3, or NFE2L2. In some embodiments, the liver cancer is associated with molecular alterations caused by viral-mediated insertional mutagenesis.

In some embodiments, the cancer is an ovarian cancer. In some embodiments, the ovarian cancer is a carcinoma. In some embodiments, the ovarian cancer is epithelial ovarian carcinoma. In some embodiments, the ovarian cancer is serous carcinoma. In some embodiments, the ovarian cancer is endometrioid carcinoma. In some embodiments, the ovarian cancer is mucinous carcinoma. In some embodiments, the ovarian cancer is clear cell carcinoma. In some embodiments, the ovarian cancer arises from germ cell tumors. In some embodiments, the ovarian cancer arises from teratomas. In some embodiments, the ovarian cancer arises from dysgerminomas. In some embodiments, the ovarian cancer arises from endodermal sinus tumors. In some embodiments, the ovarian cancer arises from stromal cell tumors. In some embodiments, the ovarian cancer is a sarcoma. In some embodiments, the ovarian cancer is ovarian sarcoma. In some embodiments, the ovarian cancer arises from Krukenberg tumors. In some embodiments, the ovarian cancer arises from ovarian cysts.

In some embodiments, the cancer is a pancreatic cancer. In some embodiments, the pancreatic cancer is an exocrine pancreatic cancer. In some embodiments, the pancreatic cancer is a carcinoma. In some embodiments, the pancreatic cancer is adenocarcinoma. In some embodiments, the pancreatic cancer is squamous cell carcinoma. In some embodiments, the pancreatic cancer is adenosquamous carcinoma. In some embodiments, the pancreatic cancer is colloid carcinoma. In some embodiments, the pancreatic cancer is acinar cell carcinoma. In some embodiments, the pancreatic cancer arises from intraductal papillary-mucinous neoplasm. In some embodiments, the pancreatic cancer arises from mucinous cystic neoplasm with an invasive adenocarcinoma. In some embodiments, the pancreatic cancer is a neuroendocrine pancreatic cancer.

In some embodiments, the cancer is a penile cancer. In some embodiments, the penile cancer is a carcinoma. In some embodiments, the penile cancer is squamous cell carcinoma. In some embodiments, the penile cancer is basal cell carcinoma. In some embodiments, the penile cancer is adenocarcinoma. In some embodiments, the penile cancer is a sarcoma.

In some embodiments, the cancer is a prostate cancer. In some embodiments, the prostate cancer is a carcinoma. In some embodiments, the prostate cancer is adenocarcinoma. In some embodiments, the prostate cancer is acinar adenocarcinoma. In some embodiments, the prostate cancer is prostatic ductal adenocarcinoma. In some embodiments, the prostate cancer is transitional cell carcinoma. In some embodiments, the prostate cancer arises from neuroendocrine tumors. In some embodiments, the prostate cancer is small cell carcinoma. In some embodiments, the prostate cancer is squamous cell carcinoma. In some embodiments, the prostate cancer is prostate sarcoma.

In some embodiments, the cancer is a skin cancer. In some embodiments, the skin cancer is a carcinoma. In some embodiments, the skin cancer is basal cell carcinoma. In some embodiments, the skin cancer is squamous cell carcinoma. In some embodiments, the skin cancer is squamous cell carcinoma in situ (e.g., Bowen disease). In some embodiments, the skin cancer arises from keratoacanthomas. In some embodiments, the skin cancer is Merkel cell carcinoma. In some embodiments, the skin cancer is sebaceous carcinoma. In some embodiments, the skin cancer arises from actinic keratoses. In some embodiments, the skin cancer is a melanoma. In some embodiments, the skin cancer is a lymphoma. In some embodiments, the skin cancer is cutaneous T-cell lymphoma. In some embodiments, the skin cancer is dermatofibrosarcoma protuberans. In some embodiments, the skin cancer is a sarcoma. In some embodiments, the skin cancer is Kaposi sarcoma. In some embodiments, the skin cancer arises from skin adnexal tumors.

In some embodiments, the skin cancer is associated with sun exposure. In some embodiments, the skin cancer is associated with excessive exposure to ultraviolet radiation or ionizing radiation. In some embodiments, the skin cancer is associated with a weakened immune system, such as in those persons with AIDS, those taking immune suppressants, or those infected by the human papilloma virus. In some embodiments, the skin cancer is associated with inherited syndromes, such as familiar melanoma, Gorlin syndrome, and xeroderma pigmentosum. In some embodiments, the skin cancer is associated with unusual moles that may be characterized by asymmetrical shapes, irregular borders, changes in color, or growth. In some embodiments, the skin cancer is associated with mutations in BRAF, TP53, CDK4, CDKN2A or MC1R. In some embodiments, the skin cancer is characterized by chromosomal instability.

In some embodiments, the cancer is an intestinal cancer. In some embodiments, the intestinal cancer is a small bowel cancer. In some embodiments, the intestinal cancer is a gastric cancer. In some embodiments, the intestinal cancer is a stomach cancer. In some embodiments, the intestinal cancer is a carcinoma. In some embodiments, the intestinal cancer is adenocarcinoma. In some embodiments, the intestinal cancer is a sarcoma. In some embodiments, the intestinal cancer arises from gastrointestinal stromal tumors. In some embodiments, the intestinal cancer arises from carcinoid tumors. In some embodiments, the intestinal cancer is a lymphoma.

In some embodiments, the intestinal cancer is associated with gastroesophageal reflux disease. In some embodiments, the intestinal cancer is associated with obesity. In some embodiments, the intestinal cancer is associated with bacterial infections, such as infection with Helicobacter pylori. In some embodiments, the intestinal cancer is associated with viral infection, such as infection by Epstein-Barr virus (human herpesvirus 4). In some embodiments, the intestinal cancer is associated with inflammatory diseases, like gastritis. In some embodiments, the intestinal cancer is associated with smoking. In some embodiments, the intestinal cancer is associated with DNA hypermethylation. In some embodiments, the intestinal cancer is associated with microsatellite instability. In some embodiments, the intestinal cancer is associated with chromosomal instability. In some embodiments, the intestinal cancer is associated with mutations in PIK3CA, TP53, CDH1, or RHOA. In some embodiments, the intestinal cancer is associated with genomically stable tumors.

In some embodiments, the cancer is a testicular cancer. In some embodiments, the testicular cancer arises from germ cell tumors. In some embodiments, the testicular cancer arises from seminomas. In some embodiments, the testicular cancer arises from seminoma germ cell tumors. In some embodiments, the testicular cancer arises from spermatocytic seminomas. In some embodiments, the testicular cancer arises from nonseminomatous germ cell tumors. In some embodiments, the testicular cancer is embryonal carcinoma. In some embodiments, the testicular cancer is yolk sac carcinoma. In some embodiments, the testicular cancer choriocarcinoma. In some embodiments, the testicular cancer arises from teratomas. In some embodiments, the testicular cancer arises from stromal tumors. In some embodiments, the testicular cancer arises from Leydig cell tumors. In some embodiments, the testicular cancer arises from Sertoli cell tumors.

In some embodiments, the cancer is a thyroid cancer. In some embodiments, the thyroid cancer is a carcinoma. In some embodiments, the thyroid cancer is papillary carcinoma. In some embodiments, the thyroid cancer is follicular carcinoma. In some embodiments, the thyroid cancer is Hurthle cell carcinoma. In some embodiments, the thyroid cancer is medullary thyroid carcinoma. In some embodiments, the thyroid cancer is anaplastic carcinoma. In some embodiments, the thyroid cancer is a lymphoma.

In some embodiments, the cancer is a tracheal cancer. In some embodiments, the tracheal cancer is a carcinoma. In some embodiments, the tracheal cancer is squamous cell carcinoma. In some embodiments, the tracheal cancer is adenoid cystic carcinoma. In some embodiments, the tracheal cancer arises from carcinoid tumors.

In some embodiments, the cancer is a urethral cancer. In some embodiments, the urethral cancer is a carcinoma. In some embodiments, the urethral cancer is squamous cell carcinoma. In some embodiments, the urethral cancer is transitional cell carcinoma. In some embodiments, the urethral cancer is adenocarcinoma.

In some embodiments, the cancer is a vaginal cancer. In some embodiments, the vaginal cancer is a carcinoma. In some embodiments, the vaginal cancer is squamous cell carcinoma. In some embodiments, the vaginal cancer is adenocarcinoma. In some embodiments, the vaginal cancer is a melanoma. In some embodiments, the vaginal cancer is a sarcoma. In some embodiments, the vaginal cancer is rhabdomyosarcoma.

In some embodiments, the cancer is a vulvar cancer. In some embodiments, the vulvar cancer is a carcinoma. In some embodiments, the vulvar cancer is squamous cell carcinoma. In some embodiments, the vulvar cancer is verrucous carcinoma. In some embodiments, the vulvar cancer is adenocarcinoma. In some embodiments, the vulvar cancer is a melanoma. In some embodiments, the vulvar cancer is a sarcoma.

In some embodiments, the cancer is a rare cancer such as dermatofibroma protuberans, angiosarcoma of the skin, non-HIV-related Kaposi's sarcoma, sebaceous cell carcinoma, and eccrine carcinoma. In some embodiments, the cancer is an immunogenic cancer or a cancer associated with or that arises from a viral infection. In some embodiments, the viral infection is caused by the Merkel cell polyoma virus. Without wishing to be bound by theory, the Merkel cell polyoma virus may be associated, at least in some instances, with Merkel cell carcinoma. In some embodiments, the viral infection is caused by the Epstein-Barr virus (e.g., associated with Burkitt's lymphoma), the human papilloma virus (e.g., associated with cervical cancer), Hepatitis B virus or Hepatitis C virus (e.g., possibly associated with some forms of hepatocellular carcinoma).

In some embodiments, the cancer (e.g., BCC, such as locally advanced BCC, metastatic BCC, and/or superficial lesions in BCC, melanoma or liver cancer, such as HCC) is characterized as microsatellite instability (MSI) high, or MSI-H, or mismatch repair deficient, or dMMR. MSI-H or dMMR cancers are characterized by defects in DNA replication, particularly in the microsatellite regions. In some embodiments, the cancer (e.g., BCC, such as locally advanced BCC, metastatic BCC, and/or superficial lesions in BCC, melanoma or liver cancer, such as HCC) is microsatellite stable, or MSI-low. The presence of MSI-H and dMMR tumors has been reported in diverse cancer types, including colon, colorectal, endometrial, biliary, gastric, gastroesophageal junction, pancreatic, small intestinal, breast, triple negative breast, prostate, bladder, esophageal, sarcoma, thyroid, retroperitoneal adenocarcinoma, small cell lung, ovarian, pancreatic, prostate, central nervous system, and non-small cell lung cancers. In some embodiments, the cancer is characterized by high tumor mutational burden (TMB). TMB is the number of non-synonymous somatic coding mutations within tumor cells, often reported as number of mutations per megabase. In some embodiments, the cancer is BCC, such as locally advanced BCC, metastatic BCC, and/or superficial lesions in BCC, having high TMB.

In some embodiments, the cancer is a metastatic cancer that expresses programmed death-ligand 1 (PD-L1) or PD-1. In some embodiments, the cancer (e.g., BCC, such as locally advanced BCC, metastatic BCC, and/or superficial lesions in BCC, melanoma or liver cancer, such as HCC) is a cancer in which the cancer cells express programmed death-ligand 1 (PD-L1).

In some embodiments, immune cells (e.g., effector T cells) associated with the cancer express PD-1. In some embodiments, the PD-1 expressed by immune cells interacts with PD-L1 expressed by cancer cells.

In some embodiments, the cancer is refractory or resistant to treatment with an immune checkpoint inhibitor antibody, such as an anti-PD-1 antibody, an anti-PD-L1 antibody, or an anti-CTLA4 antibody. In some embodiments, the cancer is not responsive to treatment with an immune checkpoint inhibitor antibody. In some embodiments, the cancer is resistant to treatment with an immune checkpoint inhibitor antibody. In some embodiments, the cancer is not responsive to treatment with an anti-PD-1 antibody, an anti-PD-L1 antibody, and/or an anti-CTLA4 antibody. In some embodiments, the cancer is resistant to treatment with an anti-PD-1 antibody, an anti-PD-L1 antibody, and/or an anti-CTLA4 antibody.

In some embodiments, the cancer (e.g., BCC, such as locally advanced BCC, metastatic BCC, and/or superficial lesions in BCC, melanoma or liver cancer, such as HCC) is refractory or resistant to treatment with an immune checkpoint inhibitor antibody, such as a PD-1 antibody.

In some embodiments, a method for treating cancer (e.g., BCC, such as locally advanced BCC, metastatic BCC, and/or superficial lesions in BCC, melanoma or liver cancer, such as HCC) comprises administering an SNA and one or both of an agonist of a TNFRSF member and an immune checkpoint inhibitor disclosed herein to a subject that has a solid tumor. A solid tumor as used herein refers to an abnormal mass of tissue that usually does not contain cysts or liquid areas. Solid tumors may be benign (not cancer), or malignant (cancer). Different types of solid tumors are named for the type of cells that form them. Non-limiting examples of solid tumors include sarcomas, carcinomas, and lymphomas. Other solid tumors arising from other tissues, organs or areas of the body are also contemplated herein. In some embodiments, the solid tumor or tumor lesion is on or near the skin, on or near soft tissue and in or near a lymph node. In some embodiments, the solid tumor or tumor lesion is on or near cutaneous soft tissue or subcutaneous soft tissue.

In some embodiments, the method for treating cancer (e.g., BCC, such as locally advanced BCC, metastatic BCC, and/or superficial lesions in BCC, melanoma or liver cancer, such as HCC) further comprises administration of an antigen. In some embodiments, the antigen is a cancer antigen. In some embodiments, the antigen is positioned on the surface of an SNA disclosed herein. In some embodiments, the antigen is linked directly to the oligonucleotide shell of an SNA. In some embodiments, the antigen is linked indirectly to the oligonucleotide shell of the SNA through a molecule. In some embodiments, the antigen is linked directly to the liposomal core of the SNA. In some embodiments, the antigen is linked indirectly to the liposomal core of the SNA through a molecule. In some embodiments, the antigen is linked or attached to or linked to a spacer. In some embodiments, the antigen is linked or attached to a spacer, and the spacer is linked or attached to the molecule. In some embodiments, an oligonucleotide (e.g., a CpG oligonucleotide) is conjugated, linked or attached to an antigen to form an antigen-oligonucleotide. In some embodiments, an antigen-oligonucleotide conjugate is linked to the SNA through hybridization to an oligonucleotide (e.g., CpG oligonucleotide) on the SNA. In some embodiments, the antigen is encapsulated in the core of an SNA disclosed herein. In some embodiments, the antigen, the SNA and one or both of the agonist of a TNFRSF member and the immune checkpoint inhibitor are administered in the same formulation. In some embodiments, the antigen, the SNA and one or both of the agonist of a TNFRSF member and the immune checkpoint inhibitor are in administered in two or more separate formulations. In some embodiments, the antigen and SNA are administered in the same formulation and one or more of the agonist of a TNFRSF member and the immune checkpoint inhibitor are in a separate formulation.

In some embodiments, the molecule is selected from the group consisting of tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, saturated C8-C22 fatty acids, saturated C8-C22 ether derivatives of glycerol, saturated and unsaturated amide derivatives of C8-C22 fatty acids and mono- and 1,2- or 1,3-di-amino glycerols, and derivatives thereof.

In some embodiments, an SNA disclosed herein is administered in conjunction or in combination with one or both of an agonist of a TNFRSF member and an immune checkpoint inhibitor disclosed herein. The terms “in conjunction with,” “in combination with,” or “co-administered” refers to a therapy which involves the delivery of two compounds, such as an SNA and an immune checkpoint inhibitor, an SNA and an agonist of a TNFRSF member, or an SNA, an agonist of a TNFRSF member and an immune checkpoint inhibitor to a subject. The two or three compounds may be administered together in a single composition, at the same time, in two or three separate compositions using the same or different routes of administration, or in two or three separate compositions at different times using the same or different routes of administration. In some embodiments, the two compounds, such as an SNA disclosed herein and one of an agonist of a TNFRSF member and an immune checkpoint inhibitor disclosed herein, are administered in separate compositions using different routes of administration at the same time or substantially the same time. In some embodiments, an SNA disclosed herein and one or both of an agonist of a TNFRSF member and an immune checkpoint inhibitor disclosed herein are administered within five days, within four days, within 72 hours, within 48 hours, within 24 hours, within 12 hours, within 6 hours, within 4 hours, within 3 hours, within 2 hours, within 1 hour, within 30 minutes, within 10 minutes, within 5 minutes, within 1 minute of administration of each other. In some embodiments, the SNA and one or both of the agonist of a TNFRSF member and the immune checkpoint inhibitor are administered substantially simultaneously or substantially at the same time (e.g., during the time the subject is receiving the immune checkpoint inhibitor). In some embodiments, (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, as disclosed herein, is administered once a day, once every three days, once a week, once every two weeks, once every three weeks, once every four weeks, once every five weeks, once every six weeks, once every seven weeks, once every eight weeks, once every nice weeks, once every 10 weeks, once every 12 weeks, once every 18 weeks, once every 24 weeks, once a month, once every two months, once every three months, once every four months, once every five months, once every six months, once every seven months, once every eight months, once every nine months, once every 10 months, once every 11 months, once a year, once every two years, once every three years, once every four years. In some embodiments, the immune checkpoint inhibitor is pembrolizumab administered every three weeks, avelumab administered every two weeks, or cemiplimab administered every three weeks.

In some embodiments, (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, is administered once a week, twice a week, or three times per week, for four weeks, six weeks, eight weeks, 10 weeks, 12 weeks, 14 weeks, 16 weeks, 18 weeks, 20 weeks, 24 weeks, one month, two months, three months, four months, five months, six months, seven months, eight months, nine months, 10 months, 11 months, one year, two years, three years, four years, five years, or six years.

In some embodiments, (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, is administered once a week, twice a week, or three times per week, for four weeks, six weeks, eight weeks, 10 weeks, 12 weeks, 14 weeks, 16 weeks, 18 weeks, 20 weeks, 24 weeks, one month, two months, three months, four months, five months, six months, seven months, eight months, nine months, 10 months, 11 months, one year, two years, three years, four years, five years, six years, seven years, eight years, nine years, or 10 years. In some embodiments, an SNA disclosed herein is administered every three weeks for four weeks, six weeks, eight weeks, 10 weeks, 12 weeks, 14 weeks, 16 weeks, 18 weeks, 20 weeks, 24 weeks, one month, two months, three months, four months, five months, six months, seven months, eight months, nine months, 10 months, 11 months, one year, two years, three years, four years, five years, six years, seven years, eight years, nine years, or 10 years.

In some embodiments, (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, is administered once a week, twice a week, or three times per week, for four weeks, six weeks, eight weeks, 10 weeks, 12 weeks, 14 weeks, 16 weeks, 18 weeks, 20 weeks, 24 weeks, one month, two months, three months, four months, five months, six months, seven months, eight months, nine months, 10 months, 11 months, one year, two years, three years, four years, five years, six years, seven years, eight years, nine years, or 10 years.

In some embodiments, the duration of the method for treating cancer (e.g., BCC, such as locally advanced BCC, metastatic BCC, and/or superficial lesions in BCC, melanoma or liver cancer, such as HCC) with (i) an SNA, (ii) an immune checkpoint inhibitor, (iii) an agonist of a TNFRSF member, (iv) an SNA and an immune checkpoint inhibitor, (v) an SNA and an agonist of a TNFRSF member; (vi) an immune checkpoint inhibitor and an agonist of a TNFRSF member, or (vii) an SNA, an immune checkpoint inhibitor, and an agonist of a TNFRSF member, as disclosed herein, is for 3 months, for six months, for nine months, for one year, for 1.5 years, for two years, for 2.5 years, for 3 years, for 3.5 years, for 4 years, for 4.5 years, for 5 years, for 5.5 years, for 6 years, for 6.5 years, for 7 years, for 7.5 years, for 8 years, for 8.5 years, for 9 years, for 9.5 years, for 10 years, for 15 years, for 20 years or more than 20 years.

In some embodiments, the SNA is administered every three weeks. In some embodiments, the SNA is administered about or at least about every four weeks, five weeks, six weeks, 2 months, three months, six months, nine months, one year, 1.5 years, two years, 2.5 years, three years, 3.5 years, four years, 4.5 years, five years, 5.5 years, or six years. In some embodiments, the immune checkpoint inhibitor disclosed herein is administered every three weeks for or at least about three months, six months, nine months, one year, 1.5 years, two years, 2.5 years, three years, 3.5 years, four years, 4.5 years, five years, 5.5 years, or six years. In a preferred embodiment, the immune checkpoint inhibitor disclosed herein is administered every three weeks for or at least about three months, six months, nine months, one year, 1.5 years, two years, 2.5 years, three years, 3.5 years, four years, 4.5 years, five years, 5.5 years, or six years.

In some embodiments, the first dose of the SNA disclosed herein is administered one day, two days, three days, four days, five days, six days, seven days, eight days, nine days, 10 days, 11 days, 12 days, 30 days, 14 days, 15 days, 16 days, one week, 1.5 weeks, two weeks, 2.5 weeks, three weeks, 3.5 weeks, four weeks, 4.5 weeks, five weeks, 5.5 weeks, six weeks, 6.5 weeks, seven weeks, 7.5 weeks, eight weeks, 8.5 weeks, nine weeks, 9.5 weeks, the weeks, 10.5 weeks, 11 weeks, 11.5 weeks, 12 weeks, three months, four months, five months, six months, seven months, eight months, nine months, 10 months, 11 months, or one year, 1.5 years, two years, 2.5 years, three years, 3.5 years, four years, 4.5 years, five years, 5.5 years, or six years, after the first dose of an immune checkpoint inhibitor disclosed herein is administered to the subject.

In some embodiments, an immune checkpoint inhibitor disclosed herein and the SNA disclosed herein are administered every three weeks for at least or about two years.

In some embodiments, the treatment of a disease (e.g., cancer) disclosed herein comprises administration of the SNA once a week for up to two weeks, followed by administration of the SNA once every three weeks, administration of the immune checkpoint inhibitor once every three weeks and administration of the agonist of a TNFRSF member once a week. In some embodiments, the treatment of a disease (e.g., cancer) disclosed herein comprises administration of the SNA once a week for up to three weeks, followed by administration of the SNA once every three weeks, administration of the immune checkpoint inhibitor once every three weeks and administration of the agonist of a TNFRSF member once a week. In some embodiments, the treatment of a disease (e.g., cancer) disclosed herein comprises administration of the SNA once a week for up to four weeks, followed by administration of the SNA once every three weeks, administration of the immune checkpoint inhibitor once every three weeks and administration of the agonist of a TNFRSF member once a week. In some embodiments, the treatment of a disease (e.g., cancer) disclosed herein comprises administration of the SNA once a week for up to five weeks, followed by administration of the SNA once every three weeks, administration of the immune checkpoint inhibitor once every three weeks and administration of the agonist of a TNFRSF member once a week. In some embodiments, the treatment of a disease (e.g., cancer) disclosed herein comprises administration of the SNA once a week for up to six weeks, followed by administration of the SNA once every three weeks, administration of the immune checkpoint inhibitor once every three weeks and administration of the agonist of a TNFRSF member once a week. In some embodiments, the treatment of a disease (e.g., cancer) disclosed herein comprises administration of the SNA once a week for up to seven weeks, followed by administration of the SNA once every three weeks, administration of the immune checkpoint inhibitor once every three weeks and administration of the agonist of a TNFRSF member once a week. In some embodiments, the treatment of a disease (e.g., cancer) disclosed herein comprises administration of the SNA once a week for up to eight weeks, followed by administration of the SNA once every three weeks, administration of the immune checkpoint inhibitor once every three weeks and administration of the agonist of a TNFRSF member once a week. In some embodiments, the treatment of a disease (e.g., cancer) disclosed herein comprises administration of the SNA once a week for up to nine weeks, followed by administration of the SNA once every three weeks, administration of the immune checkpoint inhibitor once every three weeks and administration of the agonist of a TNFRSF member once a week. In some embodiments, the treatment of a disease (e.g., cancer) disclosed herein comprises administration of the agonist of a TNFRSF member once a week in addition to treatment with the SNA on a regular schedule and/or treatment with the immune checkpoint inhibitor on a regular schedule. In some embodiments, the treatment of a disease (e.g., cancer) disclosed herein comprises administration of the agonist of a TNFRSF member once every two weeks in addition to treatment with the SNA on a regular schedule and/or treatment with the immune checkpoint inhibitor on a regular schedule. In some embodiments, the treatment of a disease (e.g., cancer) disclosed herein comprises administration of the agonist of a TNFRSF member once every three weeks in addition to treatment with the SNA on a regular schedule and/or treatment with the immune checkpoint inhibitor on a regular schedule. In some embodiments, the treatment of a disease (e.g., cancer) disclosed herein comprises administration of the agonist of a TNFRSF member once every four weeks in addition to treatment with the SNA on a regular schedule and/or treatment with the immune checkpoint inhibitor on a regular schedule.

In some embodiments, the treatment of a disease (e.g., cancer) disclosed herein comprises administration of the SNA once a week for up to two weeks, followed by administration of the SNA once every three weeks, administration of the immune checkpoint inhibitor once every three weeks and administration of the agonist of a TNFRSF member once every two weeks. In some embodiments, the treatment of a disease (e.g., cancer) disclosed herein comprises administration of the SNA once a week for up to three weeks, followed by administration of the SNA once every three weeks, administration of the immune checkpoint inhibitor once every three weeks and administration of the agonist of a TNFRSF member once every two weeks. In some embodiments, the treatment of a disease (e.g., cancer) disclosed herein comprises administration of the SNA once a week for up to four weeks, followed by administration of the SNA once every three weeks, administration of the immune checkpoint inhibitor once every three weeks and administration of the agonist of a TNFRSF member once every two weeks. In some embodiments, the treatment of a disease (e.g., cancer) disclosed herein comprises administration of the SNA once a week for up to five weeks, followed by administration of the SNA once every three weeks, administration of the immune checkpoint inhibitor once every three weeks and administration of the agonist of a TNFRSF member once every two weeks. In some embodiments, the treatment of a disease (e.g., cancer) disclosed herein comprises administration of the SNA once a week for up to six weeks, followed by administration of the SNA once every three weeks, administration of the immune checkpoint inhibitor once every three weeks and administration of the agonist of a TNFRSF member once every two weeks. In some embodiments, the treatment of a disease (e.g., cancer) disclosed herein comprises administration of the SNA once a week for up to seven weeks, followed by administration of the SNA once every three weeks, administration of the immune checkpoint inhibitor once every three weeks and administration of the agonist of a TNFRSF member once every two weeks. In some embodiments, the treatment of a disease (e.g., cancer) disclosed herein comprises administration of the SNA once a week for up to eight weeks, followed by administration of the SNA once every three weeks, administration of the immune checkpoint inhibitor once every three weeks and administration of the agonist of a TNFRSF member once every two weeks. In some embodiments, the treatment of a disease (e.g., cancer) disclosed herein comprises administration of the SNA once a week for up to nine weeks, followed by administration of the SNA once every three weeks, administration of the immune checkpoint inhibitor once every three weeks and administration of the agonist of a TNFRSF member once every two weeks.

In some embodiments, the treatment of a disease (e.g., cancer) disclosed herein comprises administration of the SNA once a week for up to two weeks, followed by administration of the SNA once every three weeks, administration of the immune checkpoint inhibitor once every three weeks and administration of the agonist of a TNFRSF member once every three weeks. In some embodiments, the treatment of a disease (e.g., cancer) disclosed herein comprises administration of the SNA once a week for up to three weeks, followed by administration of the SNA once every three weeks, administration of the immune checkpoint inhibitor once every three weeks and administration of the agonist of a TNFRSF member once every three weeks. In some embodiments, the treatment of a disease (e.g., cancer) disclosed herein comprises administration of the SNA once a week for up to four weeks, followed by administration of the SNA once every three weeks, administration of the immune checkpoint inhibitor once every three weeks and administration of the agonist of a TNFRSF member once every three weeks. In some embodiments, the treatment of a disease (e.g., cancer) disclosed herein comprises administration of the SNA once a week for up to five weeks, followed by administration of the SNA once every three weeks, administration of the immune checkpoint inhibitor once every three weeks and administration of the agonist of a TNFRSF member once every three weeks. In some embodiments, the treatment of a disease (e.g., cancer) disclosed herein comprises administration of the SNA once a week for up to six weeks, followed by administration of the SNA once every three weeks, administration of the immune checkpoint inhibitor once every three weeks and administration of the agonist of a TNFRSF member once every three weeks. In some embodiments, the treatment of a disease (e.g., cancer) disclosed herein comprises administration of the SNA once a week for up to seven weeks, followed by administration of the SNA once every three weeks, administration of the immune checkpoint inhibitor once every three weeks and administration of the agonist of a TNFRSF member once every three weeks. In some embodiments, the treatment of a disease (e.g., cancer) disclosed herein comprises administration of the SNA once a week for up to eight weeks, followed by administration of the SNA once every three weeks, administration of the immune checkpoint inhibitor once every three weeks and administration of the agonist of a TNFRSF member once every three weeks. In some embodiments, the treatment of a disease (e.g., cancer) disclosed herein comprises administration of the SNA once a week for up to nine weeks, followed by administration of the SNA once every three weeks, administration of the immune checkpoint inhibitor once every three weeks and administration of the agonist of a TNFRSF member once every three weeks.

In some embodiments, an SNA and an agonist of a TNFRSF member disclosed herein are administered to a subject with a cancer (e.g., BCC, such as locally advanced BCC, metastatic BCC, and/or superficial lesions in BCC, melanoma or liver cancer, such as HCC) that is refractory, resistant or non-responsive to therapy with an immune checkpoint inhibitor, such as an inhibitor of PD-1 (e.g., an anti-PD-1 antibody) or an inhibitor of PD-L1 (e.g., an anti-PD-L1 antibody), or both an inhibitor of PD-1 and an inhibitor of PD-L1. In some embodiments, an SNA and an agonist of a TNFRSF member disclosed herein are administered to a subject with a cancer (e.g., BCC, such as locally advanced BCC, metastatic BCC, and/or superficial lesions in BCC, melanoma or liver cancer, such as HCC) that is refractory, resistant or non-responsive to therapy with avelumab, pembrolizumab or cemiplimab. A subject with a cancer (e.g., BCC, such as locally advanced BCC, metastatic BCC, and/or superficial lesions in BCC, melanoma or liver cancer, such as HCC) that is refractory, resistant or non-responsive to therapy with an immune checkpoint inhibitor is a subject with progressive disease.

In some embodiments, an SNA disclosed herein, alone or in combination with an immune checkpoint inhibitor disclosed herein, is administered to a subject with metastatic Merkel cell carcinoma, or metastatic cutaneous squamous cell carcinoma or locally advanced cutaneous squamous cell carcinoma who is not a candidate for curative surgery or curative radiation. In some embodiments, an SNA disclosed herein, alone or in combination with an immune checkpoint inhibitor disclosed herein, is administered to a subject with locally advanced or metastatic BCC, melanoma or liver cancer (e.g., HCC). In some embodiments, the subject is not a candidate for curative surgery or curative radiation.

In some embodiments, an SNA and an agonist of a TNFRSF member disclosed herein, by themselves or in combination with an immune checkpoint inhibitor disclosed herein, are administered to a subject with a cancer (e.g., BCC, such as locally advanced BCC, metastatic BCC, and/or superficial lesions in BCC, melanoma or liver cancer, such as HCC) that is progressive (or shows the characteristic(s) of Progressive Disease (PD), as measured by the RECIST criteria and disclosed herein). PD is defined by the RECIST criteria as having at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression). In some embodiments, an SNA and an agonist of a TNFRSF member disclosed herein, by themselves or in combination with an immune checkpoint inhibitor disclosed herein, are administered to a subject with a cancer (e.g., BCC, such as locally advanced BCC, metastatic BCC, and/or superficial lesions in BCC, melanoma or liver cancer, such as HCC) that is stable (or shows the characteristic(s) of Stable Disease (SD) as measured by the RECIST criteria and disclosed herein).

In some embodiments, administration of an SNA and an agonist of a TNFRSF member disclosed herein, by themselves or in combination with an immune checkpoint inhibitor disclosed herein, to a subject with a cancer (e.g., sarcoma, melanoma, Merkel cell carcinoma, BCC (e.g., locally advanced and/or metastatic BCC), cutaneous squamous cell carcinoma, head and neck squamous cell carcinoma, mucosal melanoma) that is progressive or shows one or more characteristics of PD causes the cancer to become stable or show characteristics of SD.

In some embodiments, the cancer (e.g., BCC, such as locally advanced BCC, metastatic BCC, and/or superficial lesions in BCC, melanoma or liver cancer, such as HCC) is stable or SD for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, at least 40 weeks, at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, at least 40 months, at least one year, at least two years, at least three years, at least four years, at least five years, at least six years, at least seven years, at least eight years, at least nine years, at least 10 years, at least 11 years or at least 12 years in the subject.

In some embodiments, provided herein are methods of treatment involving administering a combination therapy of an SNA and an agonist of a TNFRSF member, by themselves or with an immune checkpoint inhibitor to an identified subject or patient having a tumor, where the subject or patient is identified by at least one of: (a) having an intact functioning immune system; (b) identifying the presence of a threshold amount of PD-L1 expression in the tumor (i.e. greater than or equal to 50% of cells being PD-L1 positive); (c) identifying the presence of a threshold amount of one or more of a T-effector associated gene and/or one or more of an interferon-γ associated gene (e.g., level of gene expression as measured in the subject by methods known to one of ordinary skill in the art is higher than a pre-determined value); and (d) meeting a threshold level of tumor mutational burden (TMB).

In some embodiments, a cancer (e.g., BCC, such as locally advanced BCC, metastatic BCC, and/or superficial lesions in BCC, melanoma or liver cancer, such as HCC) is characterized or analyzed using tumor tissue (e.g., a biopsy sample) obtained from a cancer patient or subject with cancer, image(s) of tissue sections (e.g., immunohistochemical staining), a blood sample, and gene expression. These methods can identify which subjects or patients have the greatest potential to respond to immune-mediated cancer therapy (e.g., INCAGN1949, retifanlimab, atezolizumab, pembrolizumab, ABBV-386, ABBV-181, or ipilimumab) or can be indicative of desired immune activation in a subject with cancer.

For instance, a certain level of one or more of cytokine expression, chemokine expression and immune cell activation measured in a blood sample or in a biopsy sample from a subject with cancer (e.g., sarcoma, melanoma, Merkel cell carcinoma, BCC (e.g., locally advanced and/or metastatic BCC), cutaneous squamous cell carcinoma, head and neck squamous cell carcinoma, mucosal melanoma) after administration of an SNA and an agonist of a TNFRSF member disclosed, by themselves or in combination with an immune checkpoint inhibitor disclosed, can be indicative of desired immune activation in the subject with cancer.

In some embodiments, administration of the SNA at a dose (e.g., a therapeutic dose, such as 2 mg, 4 mg, 8 mg, 16 mg or 32 mg) and the agonist of a TNFRSF member at a dose (e.g., a therapeutic dose, such as 7 mg, 20 mg, 70 mg, 200 mg, 350 mg, 700 mg, or 1400 mg), by themselves or in combination with an immune checkpoint inhibitor at a dose (e.g., therapeutic dose, such as 200 mg, 350 mg or 800 mg) results in one or more of increased cytokine expression, increased chemokine expression, increased immune cell quantity in or infiltration into one or more tumors or tumor lesions, and increased immune cell activation (e.g., CD4⁺ T cells, CD8⁺ T cells, monocytes, natural killer cells and lymphocytes) by at least or about 5%, at least or about 10%, at least or about 15%, at least or about 16%, at least or about 17%, at least or about 18%, at least or about 19%, at least or about 20%, at least or about 21%, at least or about 22%, at least or about 23%, at least or about 24%, at least or about 25%, at least or about 26%, at least or about 27%, at least or about 28%, at least or about 29%, at least or about 30%, at least or about 31%, at least or about 32%, at least or about 33%, at least or about 34%, at least or about 35%, at least or about 36%, at least or about 37%, at least or about 38%, at least or about 39%, at least or about 40%, at least or about 41%, at least or about 42%, at least or about 43%, at least or about 44%, at least or about 45%, at least or about 46%, at least or about 47%, at least or about 48%, at least or about 49%, at least or about 50%, at least or about 55%, at least or about 60%, at least or about 65%, at least or about 70%, at least or about 75%, at least or about 80%, at least or about 85%, at least or about 90%, at least or about 95%, at least or about 99%, at least about 2-fold, 3-fold, 4-fold, 5-fold, 6-fold, 7-fold, 8-fold, 9-fold, 10-fold, 15-fold, 20-fold, 25-fold, 30-fold, 40-fold, 50-fold or more, relative to a reference level.

In some embodiments, administration of the SNA at a dose (e.g., a therapeutic dose, such as 2 mg, 4 mg, 8 mg, 16 mg or 32 mg) and the agonist of a TNFRSF member at a dose (e.g., a therapeutic dose, such as 7 mg, 20 mg, 70 mg, 200 mg, 350 mg, 700 mg, or 1400 mg), by themselves or in combination with an immune checkpoint inhibitor at a dose (e.g., therapeutic dose, such as 200 mg, 350 mg, or 800 mg) results in one or more of increased cytokine expression, increased chemokine expression, increased immune cell quantity in or infiltration into one or more tumors or tumor lesions, and increased immune cell activation (e.g., CD4⁺ T cells, CD8⁺ T cells, monocytes, natural killer cells, and lymphocytes) by at least or about 100%, at least or about 150%, at least or about 2-fold, at least or about 3-fold, at least or about 4-fold, at least or about 5-fold, at least or about 6-fold, at least or about 7-fold, at least or about 8-fold, at least or about 9-fold, at least or about 10-fold, at least or about 11-fold, at least or about 12-fold, at least or about 13-fold, at least or about 14-fold, at least or about 15-fold, at least or about 16-fold, at least or about 17-fold, at least or about 18-fold, at least or about 19-fold, at least or about 20-fold, at least or about 21-fold, at least or about 22-fold, at least or about 23-fold, at least or about 24-fold, at least or about 25-fold, at least or about 26-fold, at least or about 27-fold, at least or about 28-fold, at least or about 29-fold, at least or about 30-fold, at least or about 40-fold, at least or about 50-fold, at least or about 60-fold, at least or about 70-fold, at least or about 80-fold, at least or about 90-fold, at least or about 100-fold or more, relative to a reference level.

In some embodiments, administration of the SNA at a dose (e.g., a therapeutic dose, such as 2 mg, 4 mg, 8 mg, 16 mg or 32 mg) and the agonist of a TNFRSF member at a dose (e.g., a therapeutic dose, such as 7 mg, 20 mg, 70 mg, 200 mg, 350 mg, 700 mg, or 1400 mg), by themselves or in combination with an immune checkpoint inhibitor at a dose (e.g., therapeutic dose, such as 200 mg) results in one or more of increased cytokine expression, increased chemokine expression, increased immune cell quantity in or infiltration into one or more tumors or tumor lesions, and increased immune cell activation (e.g., CD4⁺ T cells, CD8⁺ T cells, monocytes, natural killer cells, and lymphocytes) at or within the solid tumor or tumor lesion that was intratumorally injected or both at the solid tumor or tumor lesion that was intratumorally injected and a witness solid tumor or tumor lesion that was not intratumorally injected in the subject.

In some embodiments, increased immune cell quantity in or infiltration into one or more tumors or tumor lesions refers to an increased number of immune cells within the one or more tumors or tumor lesions. In some embodiments, increased immune cell quantity or immune cell infiltration is determined by measuring immune cell RNA within the one or more tumors or tumor lesions. Methods of measuring immune cell RNA within a given sample are known to those of ordinary skill in the art, and include qPCR, RNA-seq, Northern blot, and other methods. In some embodiments, increased immune cell quantity or immune cell infiltration is determined by measuring the number of immune cells within the one or more tumors or tumor lesions. Methods of measuring the number of immune cells within a given sample are known to those of ordinary skill in the art, and include flow cytometry, immunohistochemical staining, immunofluorescence staining, and other methods. In some embodiments, increased immune cell quantity or immune cell infiltration is determined by measuring immune cell protein within the one or more tumors or tumor lesions. Methods of measuring immune cell protein within a given sample are known to those of ordinary skill in the art, and include Western blot, immunohistochemical staining, immunofluorescence staining, mass spectrometry, and other methods.

In some embodiments, the cytokine or chemokine is interferon (IFN)-α, IFN-γ, interleukin (IL)-10, IL-12p40, IL-1β, IL-1RA, IL-2, IL-6, IL-8, interferon gamma-induced protein (IP)-10, monocyte chemoattractant protein (MCP)-1, or tumor necrosis factor alpha (TNF)-α. In some embodiments, the immune cell is a lymphocyte.

In some embodiments, administration of the SNA at a dose (e.g., a therapeutic dose, such as 2 mg, 4 mg, 8 mg, 16 mg or 32 mg) and the agonist of a TNFRSF member at a dose (e.g., a therapeutic dose, such as 7 mg, 20 mg, 70 mg, 200 mg, 350 mg, 700 mg, or 1400 mg), by themselves or in combination with an immune checkpoint inhibitor at a dose (e.g., therapeutic dose, such as 200 mg, 350 mg, or 800 mg) results in dose-dependent lymphocyte activation. In some embodiments, administration of the SNA at a dose (e.g., a therapeutic dose, such as 2 mg, 4 mg, 8 mg, 16 mg or 32 mg) and the agonist of a TNFRSF member at a dose (e.g., a therapeutic dose, such as 7 mg, 20 mg, 70 mg, 200 mg, 350 mg, 700 mg, or 1400 mg), by themselves or in combination with an immune checkpoint inhibitor at a dose (e.g., therapeutic dose, such as 200 mg) results in dose-dependent IL-12p40, IL-1RA, IP-10, and MCP-1 induction.

In some embodiments, the immune cell is a B-lymphocyte (e.g., All: CD3−/CD19+/CD45+; Activated B cells: CD3−/CD19+/CD45+/CD86+), a T-lymphocyte (e.g., All: CD3+/CD45+; Activated T cells: CD3+/CD45+/CD69+), a natural killer (NK) cell (e.g., All: CD3−/CD16+/CD19−/CD45+/CD56+; Activated NK cell: CD3−/CD16+/CD19−/CD45+/CD56+/CD69+), a monocyte (e.g., All: CD14+/CD45+; Activated monocytes: CD14+/CD45+/CD86+; CD14+/CD45+/CD169+), a plasmacytoid dendritic cell (e.g., All: CD14−/CD11c−/CD16−/CD19−/CD45+/CD56−/CD123+/HLA-DR+; Activated pDCs: CD11c−/CD14−/CD16−/CD19−/CD45+/CD56−/CD86+/CD123+/HLA-DR+), a myeloid dendritic cell, a classic dendritic cell, a cytotoxic T cell, or a T helper cell.

In some embodiments, the threshold amount of PD-L1 expression in the tumor is when greater than or equal to 55% of cells are PD-L1 positive, greater than or equal to 60% of cells are PD-L1 positive, greater than or equal to 65% of cells are PD-L1 positive, greater than or equal to 70% of cells are PD-L1 positive, greater than or equal to 75% of cells are PD-L1 positive, greater than or equal to 80% of cells are PD-L1 positive, greater than or equal to 85% of cells are PD-L1 positive, greater than or equal to 90% of cells are PD-L1 positive, greater than or equal to 95% of cells are PD-L1 positive, or greater than or equal to 99% of cells are PD-L1 positive. In some embodiments, the cell that is PD-L1 positive is a tumor cell. In some embodiments, the cell that is PD-L1 positive is a tumor-infiltrating immune cell. In some embodiments, the cell that is a PD-L1 positive is a tumor-infiltrating immune cell and a tumor cell.

In some embodiments, the subject is a mammal. In some embodiments, the subject is a primate. In some embodiments, the subject is a human. In some embodiments, the mammal is a vertebrate animal including, but not limited to, a mouse, rat, dog, cat, horse, cow, pig, sheep, goat, turkey, chicken, monkey, fish (e.g., aquaculture species, salmon, etc.). Thus, the disclosure herein can also be used to treat diseases or disorders, such as cancer, in human or non-human subjects.

For use in therapy, a dose of an SNA, an agonist of a TNFRSF member, or an immune checkpoint inhibitor disclosed herein is administered to a subject by any mode that delivers the SNA, TNFRSF member agonist, and/or immune checkpoint inhibitor to the desired surface (e.g., intratumoral, cutaneous, subcutaneous, nodal, systemic, etc.). The SNA, TNFRSF member agonist, and/or immune checkpoint inhibitor can be administered in a pharmaceutical composition or pharmaceutical compositions that are prepared by any means known one of ordinary skill in the art. Routes of administration include but are not limited to oral, parenteral, intramuscular, intranasal, sublingual, intratracheal, inhalation, ocular, vaginal, and rectal. In some embodiments, preferred routes of administration of an SNA, TNFRSF member agonist, or immune checkpoint inhibitor disclosed herein include intravenous (IV) injection, IV infusion, intratumoral injection, cutaneous injection, nodal injection, internal implantation, and subcutaneous injection. In some embodiments, the SNA, the TNFRSF member agonist, and the immune checkpoint inhibitor are each independently administered through at least one route of administration chosen from intratumoral administration to a solid tumor or tumor lesion, subcutaneous administration, internal implantation, and intravenous administration.

In some embodiments, the SNA is administered through at least one route of administration chosen from IT administration to a solid tumor or tumor lesion, subcutaneous administration, internal implantation, and IV administration (e.g., IV injection, IV infusion). In some embodiments, the SNA is administered through IT administration to a solid tumor or tumor lesion. In some embodiments, the SNA is administered through SC administration. In some embodiments, the SNA is administered through IV administration (e.g., IV injection, IV infusion). In some embodiments, the SNA is administered through IT administration and SC administration. In some embodiments, the SNA is administered through IT administration and IV administration (e.g., IV injection, IV infusion). In some embodiments, the SNA is administered through SC administration and IV administration (e.g., IV injection, IV infusion). In instances in which the SNA is administered through more than one route of administration, the SNA may be administered through the more than one route of administration concurrently (e.g., on the same day), or may be administered through the more than one route of administration at different times (e.g., on different days).

In some embodiments, the TNFRSF member agonist is administered through at least one route of administration chosen from IT administration to a solid tumor or tumor lesion, SC administration and IV administration (e.g., IV injection, IV infusion). In some embodiments, the TNFRSF member agonist is administered through IT administration to a solid tumor or tumor lesion. In some embodiments, the TNFRSF member agonist is administered through SC administration. In some embodiments, the TNFRSF member agonist is administered through IV administration (e.g., IV injection, IV infusion). In some embodiments, the TNFRSF member agonist is administered through IT administration and SC administration. In some embodiments, the TNFRSF member agonist is administered through IT administration and IV administration (e.g., IV injection, IV infusion). In some embodiments, the TNFRSF member agonist is administered through SC administration and IV administration (e.g., IV injection, IV infusion). In instances in which the TNFRSF member agonist is administered through more than one route of administration, the TNFRSF member agonist may be administered through the more than one route of administration concurrently (e.g., on the same day), or may be administered through the more than one route of administration at different times (e.g., on different days).

In some embodiments, the immune checkpoint inhibitor is administered through at least one route of administration chosen from IT administration to a solid tumor or tumor lesion, SC administration and IV administration (e.g., IV injection, IV infusion). In some embodiments, the immune checkpoint inhibitor is administered through IT administration to a solid tumor or tumor lesion. In some embodiments, the immune checkpoint inhibitor is administered through SC administration. In some embodiments, the immune checkpoint inhibitor is administered through IV administration (e.g., IV injection, IV infusion). In some embodiments, the immune checkpoint inhibitor is administered through IT administration and SC administration. In some embodiments, the immune checkpoint inhibitor is administered through IT administration and IV administration. In some embodiments, the immune checkpoint inhibitor is administered through SC administration and IV administration (e.g., IV injection, IV infusion). In instances in which the immune checkpoint inhibitor is administered through more than one route of administration, the immune checkpoint inhibitor may be administered through the more than one route of administration concurrently (e.g., on the same day), or may be administered through the more than one route of administration at different times (e.g., on different days).

In some embodiments, administration of an SNA is through IV administration, administration of an agonist of a TNFRSF member is through IV administration (e.g., IV injection, IV infusion), and administration of an immune checkpoint inhibitor is through IV administration (e.g., IV injection, IV infusion). In some embodiments, administration of an SNA is through IT administration, administration of an agonist of a TNFRSF member is through IV administration (e.g., IV injection, IV infusion), and administration of an immune checkpoint inhibitor is through IV administration (e.g., IV injection, IV infusion). In some embodiments, administration of an SNA is through SC administration, administration of an agonist of a TNFRSF member is through IV administration (e.g., IV injection, IV infusion), and administration of an immune checkpoint inhibitor is through IV administration (e.g., IV injection, IV infusion). In some embodiments, administration of an SNA is through IT administration and SC administration, administration of an agonist of a TNFRSF member is through IV administration (e.g., IV injection, IV infusion), and administration of an immune checkpoint inhibitor is through IV administration (e.g., IV injection, IV infusion).

In some embodiments, administration of an SNA is through IV administration, administration of an agonist of a TNFRSF member is through IT administration, and administration of an immune checkpoint inhibitor is through IV administration (e.g., IV injection, IV infusion). In some embodiments, administration of an SNA is through IT administration, administration of an agonist of a TNFRSF member is through IT administration, and administration of an immune checkpoint inhibitor is through IV administration (e.g., IV injection, IV infusion). In some embodiments, administration of an SNA is through SC administration, administration of an agonist of a TNFRSF member is through IT administration, and administration of an immune checkpoint inhibitor is through IV administration (e.g., IV injection, IV infusion). In some embodiments, administration of an SNA is through IT administration and SC administration, administration of an agonist of a TNFRSF member is through IT administration, and administration of an immune checkpoint inhibitor is through IV administration (e.g., IV injection, IV infusion).

In some embodiments, one or more of an SNA, an agonist of a TNFRSF member and an immune checkpoint inhibitor disclosed herein are administered to a deep visceral lesion (e.g., liver lesion or lung metastases). In some embodiments, the administration to a deep visceral lesion requires radiological control via computed tomography (CT) or magnetic resonance imaging (MRI). Other methods of radiological control known to one of ordinary skill in the art are also contemplated herein. In some embodiments, the administration to a deep visceral lesion requires ultrasound-guided or endoscope-guided injection and delivery. In some embodiments, the deep visceral lesion is in an internal organ of the body of the subject. In some embodiments, the deep visceral lesion is in the liver, heart, pancreas, kidney, stomach, lung, or intestines.

In some embodiments, one or more of an SNA, an agonist of a TNFRSF member and an immune checkpoint inhibitor disclosed herein are administered to a solid tumor or tumor lesion which requires detection through a method other than visualization. In some embodiments, the administration involves palpation, ultrasound imaging, computed tomography (CT), X-ray imaging, magnetic resonance imaging (MRI), or use of other imaging or detection modalities. In some embodiments, the administration involves IT injection guided by palpation, ultrasound imaging, CT, X-ray imaging, MRI, or other imaging or detection modalities. In some embodiments, the administration involves or requires other methods of radiological control known to one of ordinary skill in the art, and such methods are also contemplated herein.

In some embodiments, the immune checkpoint inhibitor and/or the agonist of a TNFRSF member is administered by IV infusion for an amount of time that is between 5 minutes and 12 hours. In some embodiments, the immune checkpoint inhibitor and/or the agonist of a TNFRSF member is administered by IV infusion for or about 5 minutes, 10 minutes, 15 minutes, 20 minutes, 31 minutes, 22 minutes, 23 minutes, 24 minutes, 25 minutes, 26 minutes, 27 minutes, 28 minutes, 29 minutes, 30 minutes, 31 minutes, 32 minutes, 33 minutes, for 34 minutes, 35 minutes, 36 minutes, 37 minutes, 38 minutes, 39 minutes, 40 minutes, 45 minutes, 50 minutes, 55 minutes, 60 minutes, 1.5 hours, two hours, 2.5 hours, three hours, 3.5 hours, four hours, 4.5 hours, five hours, 5.5 hours, six hours, eight hours, 10 hours, or 12 hours. In some embodiments, the immune checkpoint inhibitor is pembrolizumab administered by IV infusion for 30 minutes, avelumab administered by IV infusion for 60 minutes, or cemiplimab administered by IV infusion for 30 minutes. In some embodiments, the immune checkpoint inhibitor is ipilimumab administered by IV infusion for 30 minutes or 60 minutes. In some embodiments, the immune checkpoint inhibitor is nivolumab administered by IV infusion for 30 minutes or 60 minutes. In some embodiments, the immune checkpoint inhibitor and/or the agonist of a TNFRSF member is administered until disease progression or unacceptable toxicity.

In some embodiments, the immune checkpoint inhibitor is in a solution at a concentration of between 0.1 mg/mL and 50 mg/mL. In some embodiments, the immune checkpoint inhibitor is in a solution at a concentration of between 1 mg/mL and 50 mg/mL, between 5 mg/mL and 50 mg/mL, between 10 mg/mL and 50 mg/mL, between 20 mg/mL and 50 mg/mL, between 30 mg/mL and 50 mg/mL, or between 40 mg/mL and 50 mg/mL. In some embodiments, the immune checkpoint inhibitor is in a solution at a concentration of between 1 mg/mL and 45 mg/mL, between 1 mg/mL and 40 mg/mL, between 1 mg/mL and 35 mg/mL, between 1 mg/mL and 30 mg/mL, between 1 mg/mL and 25 mg/mL, between 1 mg/mL and 20 mg/mL, between 1 mg/mL and 15 mg/mL, between 1 mg/mL and 10 mg/mL, or between 1 mg/mL and 5 mg/mL. In some embodiments, the immune checkpoint inhibitor is in a solution at a concentration of between 1 mg/mL and 10 mg/mL.

In some embodiments, the immune checkpoint inhibitor is administered by IT administration (e.g., IT injection). In some embodiments, the immune checkpoint inhibitor is administered by SC administration (e.g., SC injection). In some embodiments, an SNA disclosed herein is administered IT to a cutaneous solid tumor, IT to a cutaneous tumor lesion, IT to a cutaneous target lesion, IT to a subcutaneous solid tumor or subcutaneous tumor lesion, IT to a subcutaneous target lesion, IT to a nodal solid tumor, IT to a nodal tumor lesion, or IT to a nodal target lesion. In some embodiments, an agonist of a TNFRSF member disclosed herein is administered IT to a cutaneous solid tumor, IT to a cutaneous tumor lesion, IT to a cutaneous target lesion, IT to a subcutaneous solid tumor or subcutaneous tumor lesion, IT to a subcutaneous target lesion, IT to a nodal solid tumor, IT to a nodal tumor lesion, or IT to a nodal target lesion. In some embodiments, an agonist of a TNFRSF member disclosed herein is administered IV.

In some embodiments, an SNA disclosed herein has a diameter, or a population of SNAs disclosed herein has a mean diameter of about 10 nanometers (nm) to about 150 nm. In some embodiments, the diameter or mean diameter of the SNA or population of SNAs is from about 15 nm to about 100 nm, about 20 nm to about 100 nm, about 25 nm to about 100 nm, about 15 nm to about 50 nm, about 20 nm to about 50 nm, about 10 nm to about 70 nm, about 15 nm to about 70 nm about 20 nm to about 70 nm, about 10 nm to about 30 nm, about nm to about 30 nm, about 20 nm to about 30 nm, about 10 nm to about 40 nm, about 15 nm to about 40 nm, about 20 nm to about 40 nm, about 10 nm to about 80 nm, about 15 nm to about 80 nm, or about 20 nm to about 80 nm. In some embodiments, an SNA disclosed herein has a diameter of or about 30 nm. In some embodiments, a population of SNAs disclosed herein has a mean diameter of or about 30 nm.

In some embodiments, an SNA has a diameter, or a population of SNAs has a mean diameter of about 10 nm to about 150 nm. In some embodiments, the diameter of the SNA or the mean diameter of the population of SNAs is from about 15 nm to about 100 nm, about 20 nm to about 100 nm, about 25 nm to about 100 nm, about 15 nm to about 50 nm, about 20 nm to about 50 nm, about 10 nm to about 70 nm, about 15 nm to about 70 nm about 20 nm to about 70 nm, about 10 nm to about 30 nm, about 15 nm to about 30 nm, about 20 nm to about nm, about 10 nm to about 40 nm, about 15 nm to about 40 nm, about 20 nm to about 40 nm, about 10 nm to about 80 nm, about 15 nm to about 80 nm, or about 20 nm to about 80 nm.

A “liposome core” or “liposomal core” are used interchangeably and refer to the core of an SNA disclosed herein, which is a liposome or which is liposomal in nature. In some embodiments, the core (e.g., liposome core) of an SNA disclosed herein has a diameter, or the cores (e.g., liposomal cores or liposome cores) of a population of SNAs disclosed herein, has a mean diameter of about 10 nm to about 150 nm. In some embodiments, the diameter of the core (e.g., liposomal core or liposome core) of an SNA disclosed herein or mean diameter of the cores (e.g., liposomal cores or liposome cores) of a population of SNAs disclosed herein is from about 15 nm to about 100 nm, about 20 nm to about 100 nm, about 25 nm to about 100 nm, about 15 nm to about 50 nm, about 20 nm to about 50 nm, about 10 nm to about 70 nm, about 15 nm to about 70 nm, about 20 nm to about 70 nm, about 10 nm to about nm, about 15 nm to about 30 nm, about 20 nm to about 30 nm, about 10 nm to about 40 nm, about 15 nm to about 40 nm, about 20 nm to about 40 nm, about 10 nm to about 80 nm, about 15 nm to about 80 nm, or about 20 nm to about 80 nm. In some embodiments, an SNA disclosed herein has a diameter of or about 20 nm to about 40 nm. In some embodiments, a population of SNAs disclosed herein have a mean diameter of or about 20 nm to about 40 nm.

In some embodiments, the core (e.g., a liposomal core or liposome core) of an SNA disclosed herein has a diameter, or the cores (e.g., liposomal cores or liposome cores) of a population of SNAs disclosed herein have a mean diameter of about or less than about 10 nm, nm, 20 nm, 25 nm, 30 nm, 35 nm, and/or 40 nm, or any range or combination thereof.

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

In order that the invention described herein may be more fully understood, the following examples are set forth. The examples described in this application are offered to illustrate the compounds, pharmaceutical compositions, and methods provided herein and are not to be construed in any way as limiting their scope.

### EXAMPLES

### Example 1. Synopsis

CpG-7909-SNA is a spherical nucleic acid (SNA) configuration of a toll-like receptor (TLR) 9 agonist oligonucleotide comprising the nucleotide sequence: 5′-T*C*G*T*C*G*T*T*T*T*G*T*C*G*T*T*T*T*G*T*C*G*T*T-3′/HEG/HEG/TEG Cholesteryl Ester/ (SEQ ID NO: 1), wherein the C is 2′-deoxy-P-cytidylyl, G is 2′-deoxy-P-guanylyl, T is 2′-deoxy-P-thymidylyl, HEG is Hexa(ethylene glycol)phosphodiester, TEG Cholesteryl Ester is (N-cholesteryl-3-aminopropyl)-triethyleneglycol-glyceryl-1-O-phosphodiester and * represents a phosphorothioate internucleotide linkage.

CpG-7909-SNA is a medicinal product for the treatment of advanced solid tumors. SNA of CpG oligonucleotides exhibits a suite of mechanistic and functional properties that make it ideal for agonizing TLR9. First, SNAs are more efficiently taken into and concentrated in endosomes compared to linear oligonucleotides (i.e., oligonucleotides not in the SNA configuration). Second, the oligonucleotides delivered as a part of SNAs cause an enhanced cytokine response, both in magnitude and duration, compared to linear oligonucleotides. Third, the SNA projects its oligonucleotides outward, allowing it to act upon TLRs directly. This arrangement of nucleic acids in the SNA is in contrast to other oligonucleotide delivery systems where the oligonucleotides are held internally, for example, inside virus-like particles.

In preclinical mouse models of anti-PD-1-resistant tumors, such as the breast EMT-6 model, anti-PD-1 antibodies showed no activity, whereas the combination of SNA of CpG oligonucleotides and anti-PD-1 antibodies demonstrated significant tumor growth inhibition and prolonged mouse survival. Further, SNA of CpG oligonucleotides outperformed linear oligonucleotides at the same dose.

Four dose levels of CpG-7909-SNA have been explored in a phase 1a first-in-human healthy volunteer study. Single doses were administered at 5-18.8 μg/kg subcutaneously (SC). There were no dose limiting toxicities (DLTs) and no serious adverse events (SAEs) in the study. SNA of CpG oligonucleotides was undetectable in serum at all dose levels tested.

### Dosage and Administration

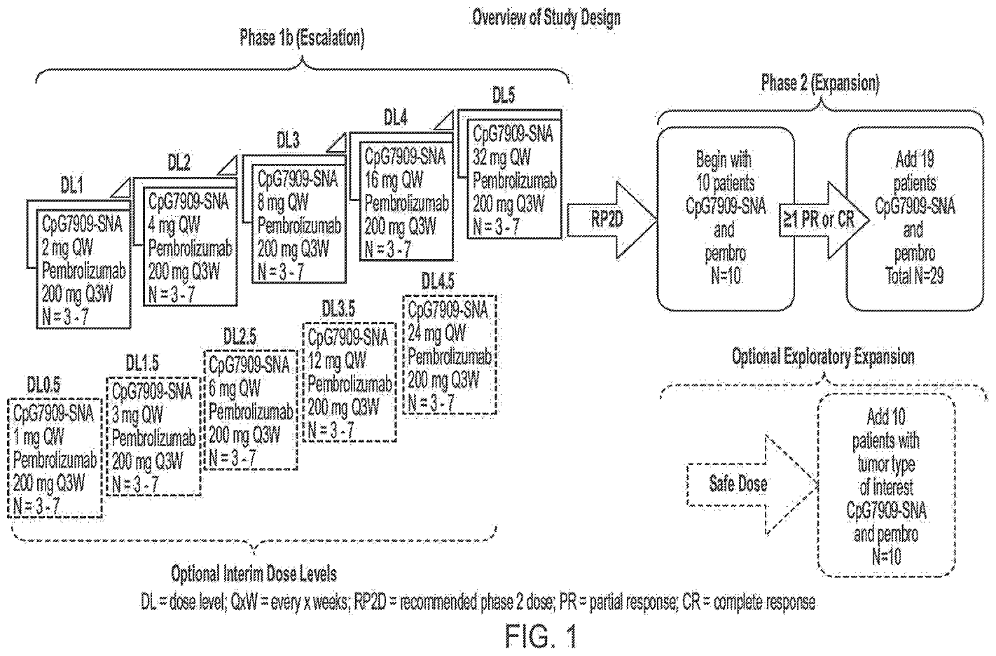

CpG-7909-SNA will be administered intratumorally (IT) into a maximum of 4 accessible lesions per administration, which are amenable to repeat administration, in cohorts of escalating flat doses of 2, 4, 8, 16, and 32 mg starting on day 1 of cycle 1. One witness lesion must remain un-injected throughout the study. Injection of deep visceral lesions is not permitted, but these may be considered target lesions for efficacy evaluation. Intermediate/de-escalation dose levels such as 1, 3, 6, 12, and 24 mg may also be explored (see FIG. 1).

Cycle 1 (CpG-7909-SNA monotherapy) will be 2 weeks long and comprise IT injections on days 1 and 8. All other cycles will be 3 weeks long. For cycles 2 and 3, CpG-7909-SNA will be administered on a weekly basis, on days 1, 8, and 15 of each 3-week cycle. Starting at cycle 4, CpG-7909-SNA will be administered every three weeks starting on day 1 until lack of clinical benefit or disease progression.

Pembrolizumab will be administered at a dose of 200 mg as an intravenous (IV) infusion over 30 minutes, on the same working day as the CpG-7909-SNA injection, every 3 weeks starting at cycle 2 day 1, as per the USPI [Keytruda® USPI].

### Study Design

The study is a classical 3+3 design, ascending dose, phase 1b study of CpG-7909-SNA combined with pembrolizumab in cancer patients. Patients will be dosed twice with CpG-7909-SNA as a monotherapy before adding pembrolizumab, which will be added starting at the second cycle. FIG. 1 shows the overall study design.

### Example 2. Dose, Route, Dose Increments, and Flat Dosing

The starting dose of CpG-7909-SNA for this trial in advanced cancer patients is 2 mg/week (equivalent to 0.031 mg/kg/week for a 65-kg patient). The systemic and local no-observed-adverse-effect levels (NOAEL(s)) defined in the 5-week monkey study using SC administration with weekly Cp7909G-SNA dosing were 3.6 and 1.2 mg/kg/week, respectively, which are 116-fold and 39-fold higher, respectively, than the proposed starting dose level. Similarly, for the 5-week rat study with weekly CpG-7909-SNA dosing, the overall NOAEL for SC was 4.5 mg/kg/week, which is 145-fold higher than the proposed starting dose level.

Importantly, it is appropriate to extrapolate the NOAEL dose levels in animals on a mg/kg basis rather than applying body surface area-based (BSA-based) scaling, because the latter paradigm is suitable only to cytotoxic anticancer agents that are metabolized much more quickly than CpG-7909-SNA, mainly by the cytochrome P450 system, in rodents vs. higher species. In contrast to the species differences in NOAELs observed for cytotoxics, which give much higher NOAELs in rodents, oligonucleotides typically exhibit similar or lower NOAELs in rodents vs. higher species. For the CpG-7909-SNA program, the NOAEL in rats was only slightly higher than in monkeys. Essentially, there are no substantial differences in the toxicity between rats and monkeys administered CpG-7909-SNA, which undermines support for BSA-based scaling and points towards the use of body weight-based extrapolation to humans. Furthermore, the area under the plasma concentration-time curve (AUC) values for rats given 1 mg/kg/week were approximately 2-fold greater than the AUC for monkeys given a similar dose level of 0.75 mg/kg/week, which again is inconsistent with the pattern observed for cytotoxic anticancer agents where the drug is cleared much more quickly by rodents, thereby further strengthening the view that BSA-based scaling is irrelevant for determining the safety margin for CpG-7909-SNA. Application of BSA-based scaling to define the human equivalent doses would falsely reflect a lesser margin of safety for the proposed starting dose level and/or dictate a starting dose level that is below the pharmacologic range for CpG-7909-SNA, which is considered suboptimal in cancer patients.

The proposed design uses IT dosing of CpG-7909-SNA to achieve high drug levels at the target site. There are no drug disposition data from animals given IT injections of CpG-7909SNA. However, in the proposed clinical study, the CpG-7909-SNA will be injected into palpable or superficial, mainly subcutaneous tumors, so the absorption and distribution of drug is expected to conform reasonably closely to what was characterized with SC dosing in animals and humans. In addition, several TLR9 agonists have been administered IT with no unexpected toxicity increases compared to prior SC administration, apart from local AEs caused by the procedure itself [Diab 2017, Milhem 2018, Ribas 2018].

The only clinical study conducted to date with SNA of CpG oligonucleotides has been a first-in-human phase 1a study (CpG-7909-SNA) in healthy volunteers. Four dose levels of CpG-7909-SNA, at 5, 10, 12.5, and 18.8 μg/kg have been evaluated. Each completed cohort included four subjects, and all received a single dose of CpG-7909-SNA SC, for a total of 16 subjects exposed. The highest total single dose of CpG-7909-SNA to any subject has been 1.4 mg. Adverse events (AEs) observed were generally mild to moderate, and no DLTs or SAEs have been reported. All AEs resolved. The expected PD effects were seen at all dose levels, while PK was below the lower limit of detection, again as expected for a TLR9 agonist given SC at low dose levels.

The pharmacophore of CpG-7909-SNA drug substance is identical to CpG-7909, which is an immunostimulatory oligonucleotide that was developed for non-small cell lung cancer and other indications through phase 3 trials. At least 12 clinical studies have been conducted with CpG-7909 (not in the SNA format) [Kreig 2012]. The highest dose of CpG-7909 reported in the literature is 0.81 mg/kg, corresponding to a dose of 53 mg in a 65 kg patient [Thompson 2009]. In this study, 40 patients were dosed and an MTD was not reached. One patient who received 0.54 mg/kg had DLTs of G3 non-hematologic AEs, including anorexia. The most commonly reported AEs were flu-like symptoms and local injection-site reactions of mild-to-moderate severity. The most commonly reported serious AE was G3 fatigue in 4 patients (10%). Grade 4 AEs included anemia, exacerbated dyspnea, and polyarthralgia in 1 patient each. CpG-7909 development was discontinued for lack of efficacy. Indeed, simply activating the innate immune system is insufficient to produce an anti-tumor response. Immune checkpoint inhibition is also needed to prevent the tumor from evading the immune response.

A flat dosing schedule is described in the current study, which represents a change from the phase 1a study. In the phase 1a study of CpG-7909-SNA in healthy volunteers (CpG-7909-SNA), the concentration of 12 circulating cytokines was measured at multiple timepoints after the administration of CpG-7909-SNA. An analysis of expression of a number of the individual cytokines vs. the dose denominated in mg/kg or the flat dose in mg revealed, quite surprisingly, that the flat dose was more predictive of the peak cytokine concentration observed in the subject plasma. Further, flat doses more accurately predicted the peak NK and pDC cell activation when compared to body mass denominated doses.

### Monotherapy DLT Period

A 2-week DLT period for the monotherapy portion of the study is justified based on the AE and PD data from the CpG-7909-SNA-101 study, which show that AEs and PD effects of CpG-7909-SNA resolve by day 5, suggesting that weekly dosing is acceptable. In addition, two administrations of CpG-7909-SNA can be performed in a 2-week period, to allow for detection of potential cumulative effects, although these have not been seen with either the pharmacophore or with other TLR9 agonists. The PK of other TLR9 agonists (short maximum observed plasma concentration [Cmax] and terminal elimination half-life [t %] with recovery to baseline levels by 24 hours) also supports a 2-week DLT period for the monotherapy.

Good tolerability was seen in the phase 1a healthy volunteer study, with no DLTs or SAEs (see Table 1 below).

TABLE 1
Percent of related AEs in a healthy volunteer phase 1a single SC dose study

| CTCAE | CpG-7909-SNA Dose Level | | | | | | | | | | | |
|---|---|---|---|---|---|---|---|---|---|---|---|---|
| V4.03 worst | 18.8 μg/kg | | | | 12.5 μg/kg | | | 10 μg/kg | | 5 μg/kg | | |
| grade, | (n = 4) | | | | (n = 4) | | | (n = 4) | | (n = 4) | | All |
| % subjects | G1 | G2 | G3 | G4 | G1 | G2 | G3 | G1 | G2 | G1 | G2 | (n = 16) |
| Any AE | 100 | 25 | 25 | 25 | 100 | 50 | 25 | 100 | 50 | 100 | 50 | 100 |
| Flu-like symptoms | 100 | — | 2 | — | 5 | 25 | — | 75 | 25 | — | — | 56 |
| Temperature* | 100 | — | — | — | 2 | 25 | — | 50 | 25 | — | — | 50 |
| Sinus tachycardia* | — | — | — | — | 5 | — | — | 25 | — | — | — | 6 |
| Injection site reaction | 100 | — | — | — | 100 | — | — | 100 | 5 | 100 | — | 100 |
| Lymphadenopathy | 100 | — | — | — | 75 | — | — | 25 | 2 | 100 | — | 75 |
| Neutropenia | — | 25 | 5 | 25 | — | 50 | 5 | 25 | 25 | 50 | 50 | 63 |
| Lymphopenia | 75 | — | 25 | — | 50 | 25 | 25 | 25 | 50 | 5 | 50 | 81 |
| Muscle twitching | — | — | — | — | — | — | — | — | — | 25 | — | 6 |
| Eye pain | — | — | — | — | — | — | — | — | — | 25 | — | 6 |
| Headache | — | — | — | — | — | — | — | — | — | 25 | — | 6 |
| Hyperesthesia | 25 | — | — | — | — | — | — | — | — | — | — | 6 |

### Example 3. Description of Overall Method

An open-label, two-part, phase 1 b/2 dose-finding study designed to determine the safety, tolerability, pharmacokinetics (PK), pharmacodynamics (PD), and proof-of-concept efficacy of IT SNA of CpG oligonucleotides alone and in combination with pembrolizumab in patients with advanced solid tumors considered amenable to anti-PD-1 therapy is provided. The study consists of a dose-escalation phase followed by dose expansion.

FIG. 1 shows the overall study design, including planned dose escalation cohorts. The study uses a classical 3+3 dose escalation design, with ascending doses of CpG-7909-SNA and enrollment of 3 patients per cohort and expansion to 6 patients in the event of a DLT. There will be two DLT periods for each patient: a monotherapy DLT period (15 days) and a combination DLT period of 22 days. AEs will be assessed per the Common Terminology Criteria for Adverse Events (CTCAE) v5.0, except for cytokine release syndrome, which will have its own grading system per Lee 2014.

Patients must have at least two target lesions evaluable per Response Evaluation Criteria in Solid Tumors (RECIST) 1.1. Patients must agree to provide a newly obtained biopsy of injected and witness lesions (that can be biopsied based on the investigator's assessment) prior to starting study treatment, and to repeat biopsies twice during study treatment, and to providing the acquired tissue for biomarker analysis. One witness lesion must remain un-injected throughout the study.

Patients will be dosed twice with CpG-7909-SNA as a monotherapy before adding pembrolizumab, which will be added starting at the second cycle. Once the MTD or highest escalation cohort has been reached, or notable efficacy has been observed at a given dose level, and a decision as to recommended phase 2 dose (RP2D) has been taken, a two-stage expansion cohort will be initiated in patients with advanced solid tumors.

The study is a 3+3 design, ascending dose, phase 1b study of IT CpG-7909-SNA combined with pembrolizumab in cancer patients. There will be two DLT periods for each patient: a monotherapy DLT period of 15 days (Cycle 1) and a combination DLT period of 22 days (Cycle 2).

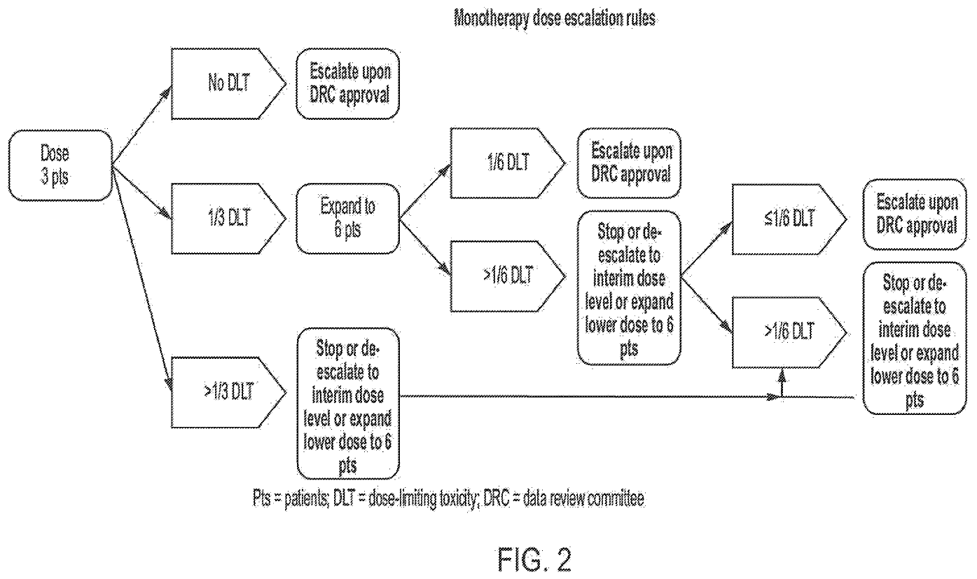

Each escalation cohort will first recruit one patient to receive CpG-7909-SNA. After 7 days, the patient's AEs and lab values will be assessed and, in the absence of DLT, that patient will continue with another weekly dose of CpG-7909-SNA to complete the 15-day monotherapy DLT period. At the time the first patient progresses to their second IT injection of CpG-7909-SNA, two additional patients can be recruited to the cohort. Therefore, patients 2 and 3 can be dosed starting on day 8 of the first treated patient. In addition, if 6 patients are required in a cohort there is no delay required for those patients to enter the study. These patients will also be assessed for early toxicity at day 7, prior to their second doses of SNA of CpG oligonucleotides. Once a patient has received 15 days of therapy, in the absence of DLT, combination therapy may begin for that patient. Once enough patients receive SNA of CpG oligonucleotides monotherapy (i.e., three patients in the absence of any DLT and six in the event of one DLT), dose escalation may proceed to a new cohort upon agreement of the data review committee (DRC). See FIG. 2 for the monotherapy dose escalation rules.

For consideration of the MTD, any potential autoimmune AE≥G2 or any chronic G≥2 toxicity thought to be related to study drug(s) will also be considered.

During the monotherapy portion of the escalation, if 1/3 patients experience DLT, the cohort will expand to six patients. If the cohort expansion results indicate that 1/6 patients have DLT, escalation may proceed. If >2/6 patients have DLT, and if only three patients were evaluated in the dose cohort below, the cohort below will be expanded to six patients. Alternatively, if >1 DLT is seen at any dose level, the DRC may decide to deescalate to an intermediate dose level, such as 24, 12, 6, 3, or 1 mg in an optional interim dose level if deemed appropriate. Planned and optional dose levels are in presented in Table 2 and Table 3.

TABLE 2
Planned dose levels

| Planned | Planned |
|---|---|
| CpG-7909-SNA Dose Level | CpG-7909-SNA Dose (mg) |
| 1 | 2 |
| 2 | 4 |
| 3 | 8 |
| 4 | 16 |
| 5 | 32 |

TABLE 3
Optional interim dose levels

| Optional Interim | |
|---|---|
| CpG-7909-SNA Dose level | CpG-7909-SNA Dose (mg) ^{a} |
| 0.5 | 1 |
| 1.5 | 3 |
| 2.5 | 6 |
| 3.5 | 12 |
| 4.5 | 24 |

^{a} The interim CpG-7909-SNA dose level may be evaluated if CpG-7909-SNA is not tolerated at a planned dose. The interim CpG-7909-SNA dose will be between the intolerable dose and the previous planned dose level.

If the maximum tolerated dose (MTD) is identified with inclusion of only 3 patients (e.g., the first dose level has >1 DLT and the de-escalation dose level has 0 DLTs), a further 3 patients should be enrolled to confirm safety prior to starting the expansion cohort. Once all feasible cohorts have been explored, AE and lab data from further cycles of treatment outside the DLT period will be considered in the selection of the RP2D/MTD, with particular attention paid to potential autoimmune effects, late toxicities seen in the 90-day follow-up period, and the relative dose intensity of CpG-7909-SNA and pembrolizumab (meaning actual/planned dose per cycle).

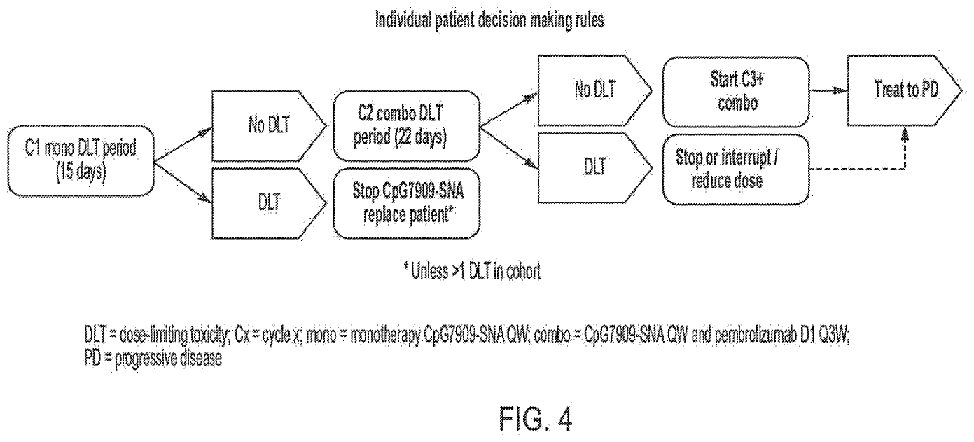

Any patients who experience DLT on CpG-7909-SNA alone will discontinue therapy and not receive pembrolizumab. Because six patients will be required for the assessment of combination therapy DLTs, if one patient experiences monotherapy DLT, another patient will be added and the cohort will be expanded to a total of 7 patients (see FIG. 4). After day 15, patients will receive weekly CpG-7909-SNA doses for the next two cycles, and pembrolizumab every three weeks until disease progression/lack of clinical benefit or discontinuation for AEs. For cycles four and beyond, both CpG-7909-SNA and pembrolizumab will be administered on day 1 of each cycle.

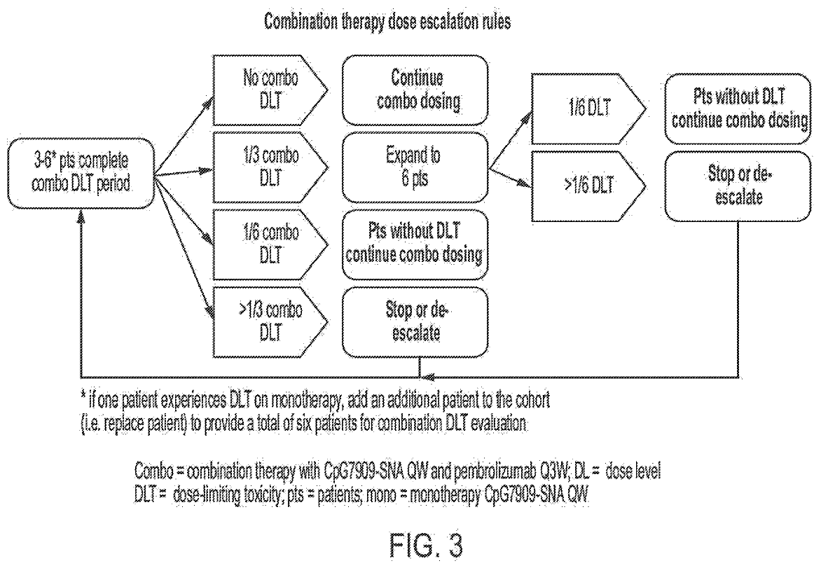

Patients will be assessed for DLTs during their first cycle of combination therapy and the same rules will be applied for de-escalation (see FIG. 3). If an MTD is identified with inclusion of only 3 patients, a further 3 patients should be enrolled to confirm safety prior to starting the expansion cohort.

If a suspected DLT occurs, a DRC meeting will be held as quickly as possible to make a judgement on the occurrence of the DLT. In the meantime, dosing of the ongoing patients in that cohort, including the dosing of pembrolizumab, will continue unless there is reason to suspect there is an unacceptable safety risk based on the nature and/or severity of the observed DLT.

If an individual patient experiences DLT in combination with pembrolizumab (Cycle 2), that patient will discontinue the study drug, unless that DLT can be managed per the Keytruda® label and the benefit is thought to outweigh the risk for that individual (see FIG. 4).

In this study, an SNA of CpG oligonucleotides DLT is defined and occurring within 15 days of starting treatment together with a reasonable chance the AEs are related to the study drug based upon the determination of the investigator (and subsequently the DRC).

A combination DLT is defined as above, but the AE relationship may be to either study drug. The combination DLT period is from days 1 to 22 of cycle two.

### Example 4. Progress in Patients with Solid Tumors and Initial Efficacy in Patients with Merkel Cell Carcinoma

Phase 1b/2 dose escalation shows safety and tolerability. There are preliminary signs of efficacy observed in Merkel Cell Carcinoma (MCC) patients previously refractory to immune checkpoint inhibitor therapy. Phase 1b/2 clinical trial in patients with solid tumors evaluates AST-008, which is an SNA consisting of TLR9 agonist designed for immuno-oncology application, in combination with pembrolizumab in patients previously refractory to immune checkpoint inhibitors. The primary objective of the dose escalation portion of the study is to evaluate the safety, tolerability, PK, and PD of AST-008 alone and in combination with pembrolizumab, and to produce a recommended Phase 2 dose. No treatment-related serious adverse events (SAEs) or dose limiting toxicities (DLTs) have been observed. Fourteen patients have been enrolled and dosed with AST-008. The final planned dose cohort is being enrolled.

The study has enrolled five melanoma patients, four MCC patients, two cutaneous squamous cell carcinoma (CSCC) patients, two head and neck squamous patients, and one mucosal melanoma patient. Most patients had progressive disease on anti-PD-(L)1 antibodies prior to enrolling.

The data demonstrate that AST-008 administration alone or in combination with pembrolizumab produces cytokine and chemokine expression and immune cell activation in patient blood indicative of desired immune activation. Furthermore, one MCC patient that previously progressed on anti-PD-1 antibody therapy has confirmed stable disease in excess of twelve weeks with decreased target lesion diameters, while a second MCC patient experienced a target lesion complete response and a confirmed overall partial response with over 24 weeks duration. Among the additional patients already enrolled, two have yet to be evaluated for efficacy, one was not evaluable, and the remaining patients had progressive disease.

The initial responses observed in patients is highly encouraging, given that no second line therapies are approved for patients with MCC. Any responses seen in these patient populations is significant. Based on these early results showing positive biomarker data and initial responses, patients resistant to anti PD-1/PD-L1 therapy are to be investigated in a Phase 2 study in MCC and in cutaneous squamous cell carcinoma (CSCC).

### Example 5. Studies of PD-1/OX40 Combination Therap in EMT6 and S180 Tumor Models

To evaluate the effects of combination therapy using anti-PD-1 and anti-OX40 antibodies in mouse models of cancer, studies were conducted using mouse models of breast cancer and sarcoma.

Tumor cells were injected in the right flank on approximately day −7 (D−7) and in the left flank on D-4. When mean tumor volume of the first-inoculated tumor was ˜90 mm³ animals were randomized into groups and treatment was initiated. The day of treatment initiation was designated day 0 or D-0.

Tumor size was monitored twice weekly until combined tumor volume reached >2000 mm³, when animals were euthanized.

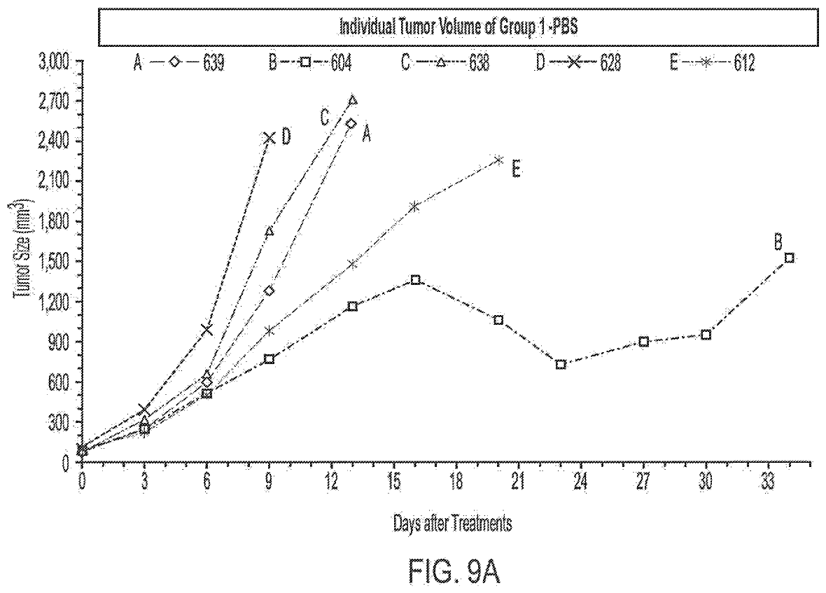

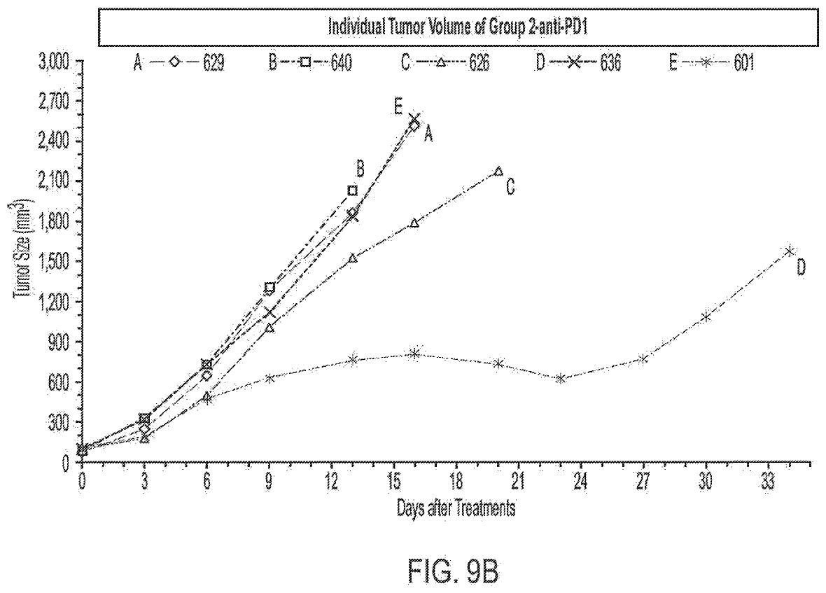

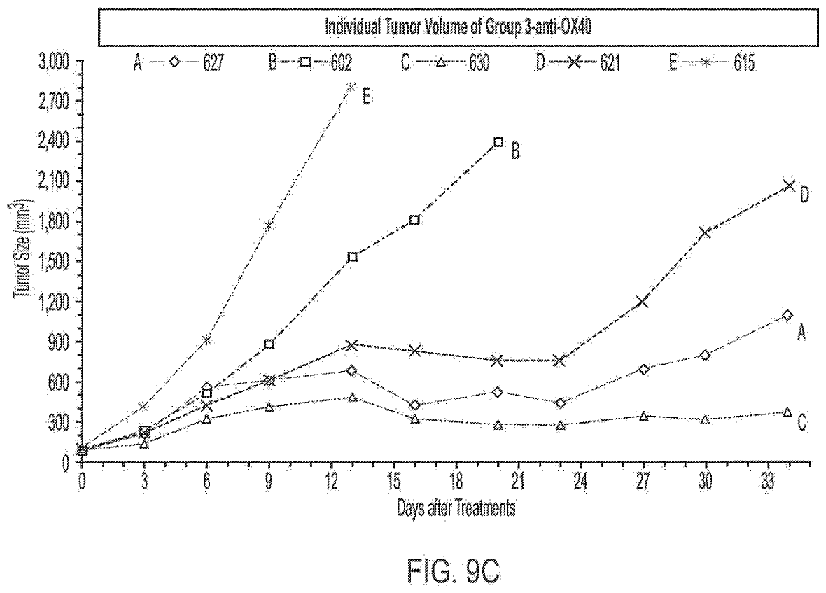

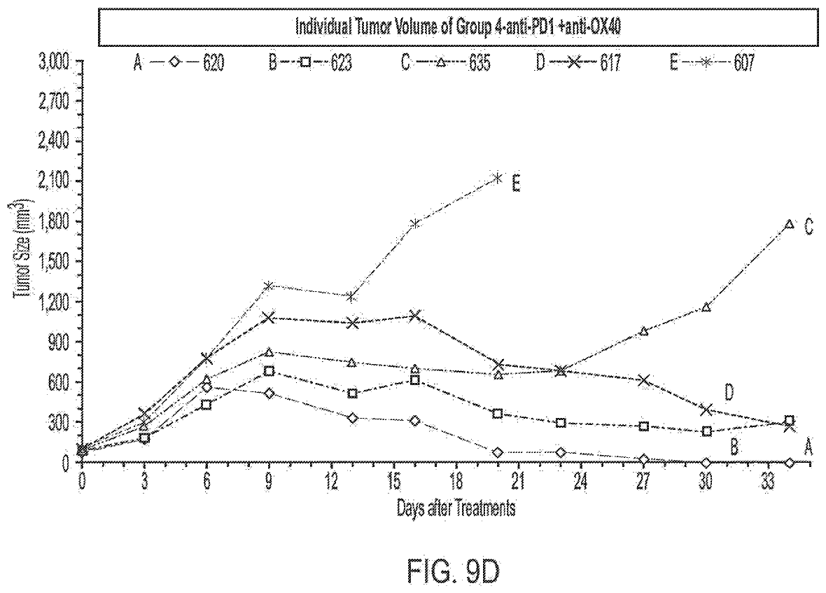

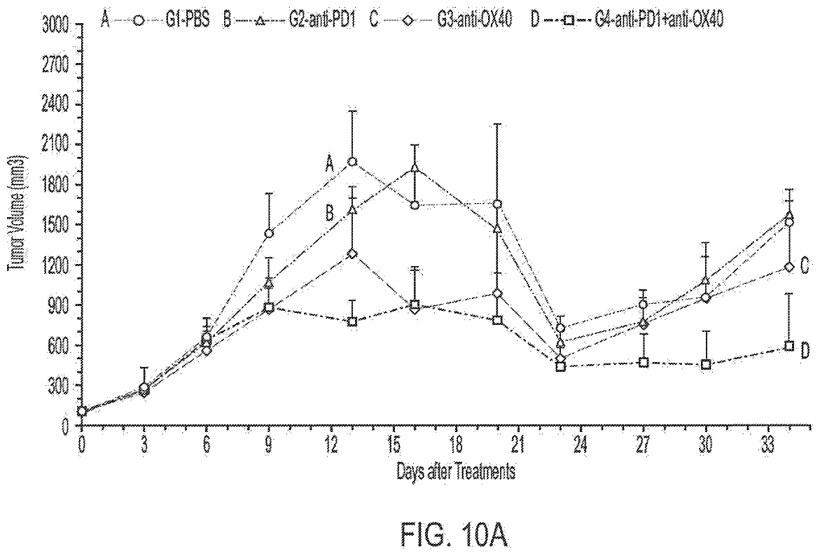

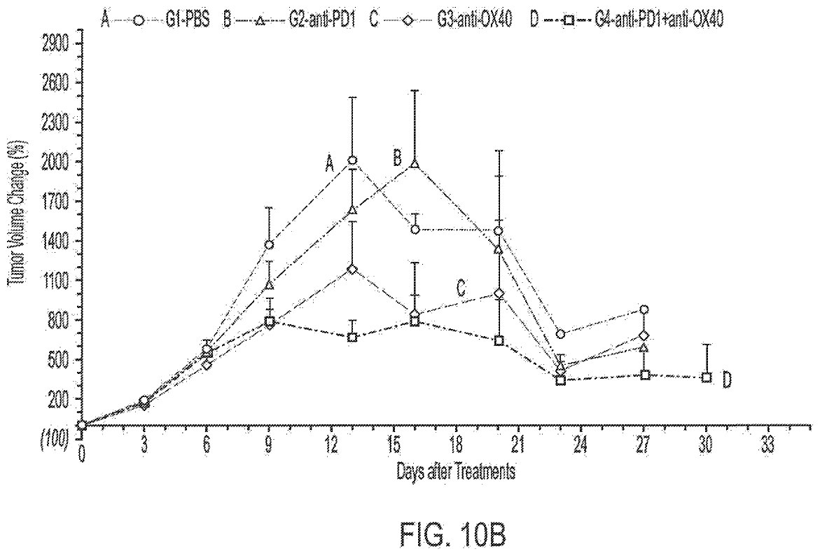

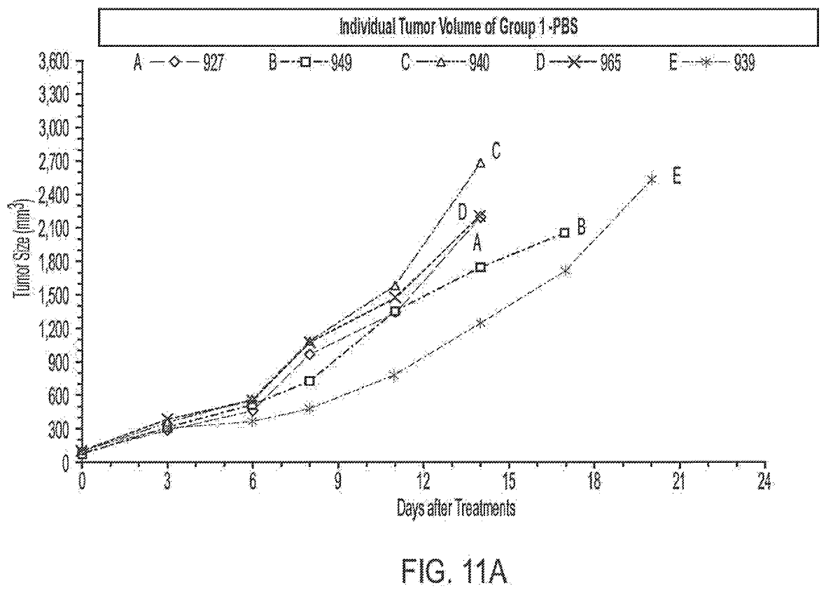

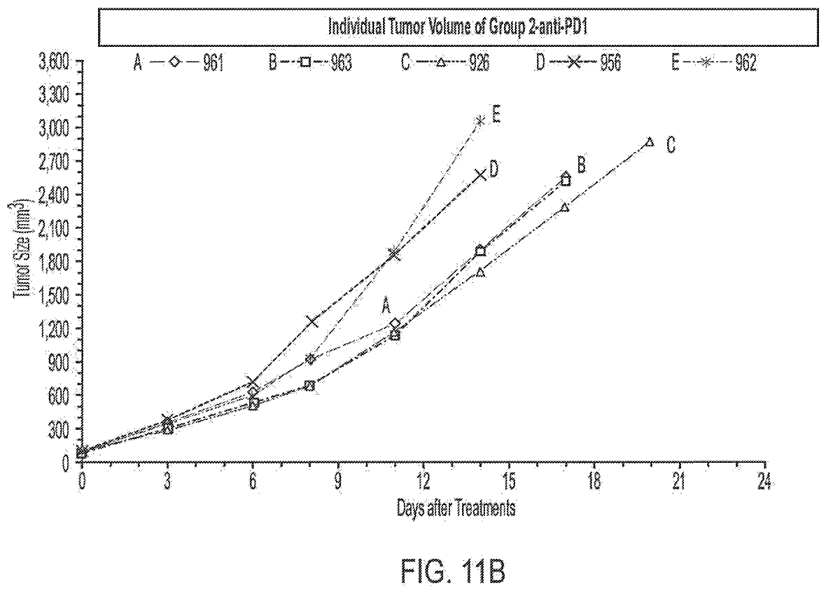

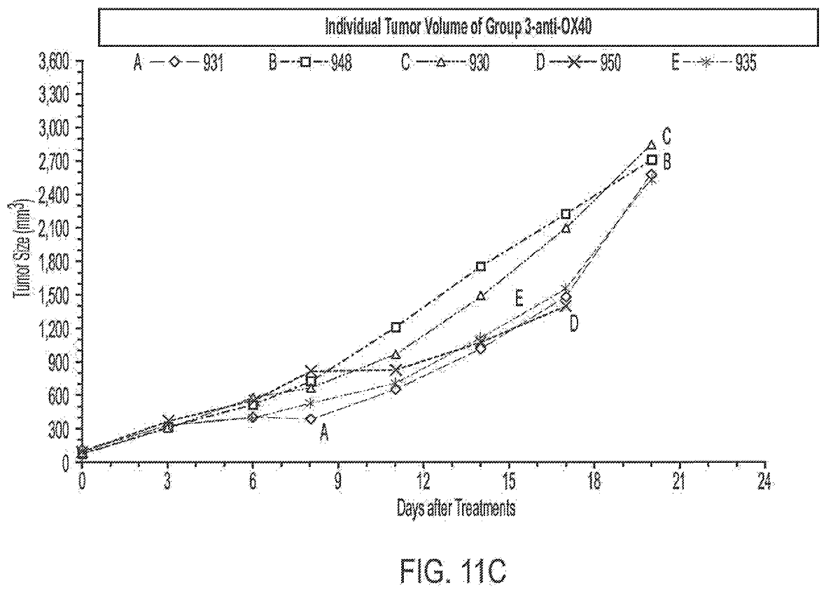

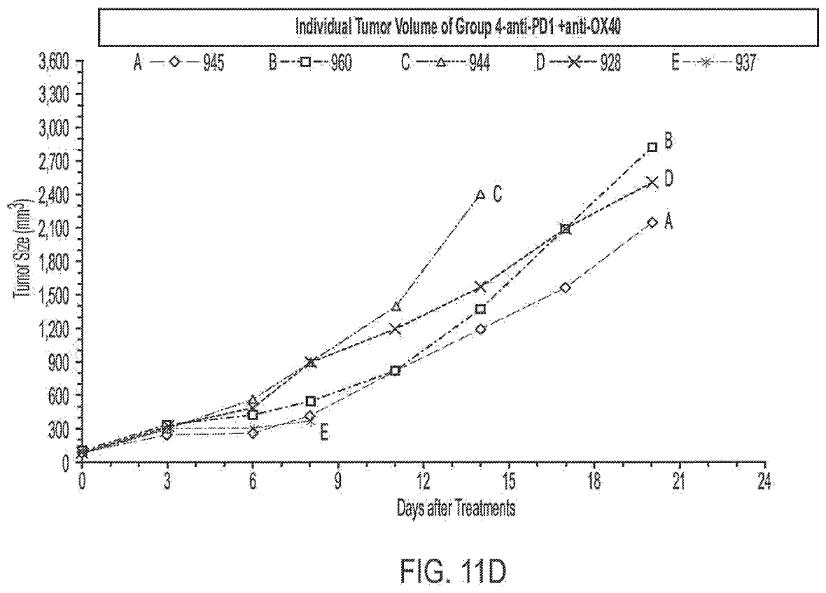

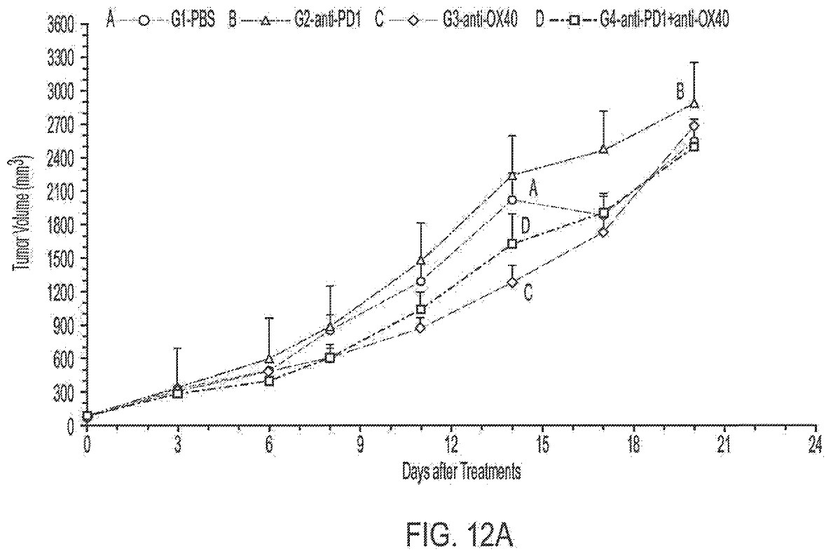

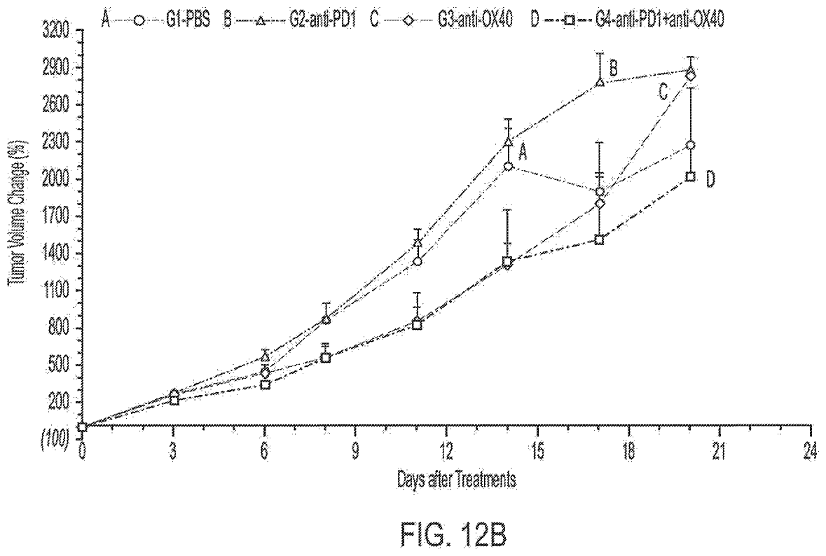

Tumor-bearing mice were injected with PBS, anti-OX40 (5 mg/kg intraperitoneally on days 0, 2, and 6), anti-PD-1 (5 mg/kg intraperitoneally on days 6, 8, and 10), or anti-OX40 and anti-PD-1. FIGS. 9A-9D show combined EMT6 (breast cancer) tumor volume of right and left flank tumors in individual animals. FIG. 10A shows the group-wise mean tumor volume of the 5 animals per group shown in FIGS. 9A-9D, and FIG. 10B shows the mean group-wise percent change in tumor volume from day 0. FIGS. 11A-11D show combined S180 (sarcoma) tumor volume of right and left flank tumors in individual animals. FIG. 12A shows the group-wise mean tumor volume of the 5 animals per group shown in FIGS. 11A-11D, and FIG. 12B shows the mean group-wise percent change in tumor volume from day 0. Combination therapy using anti-PD-1 and anti-OX40 was more effective than either single agent alone in the S180 tumor model.

### Example 6

CpG-7909-SNA is a spherical nucleic acid (SNA) configuration of a toll-like receptor (TLR) 9 agonist oligonucleotide comprising the nucleotide sequence: 5′-T*C*G*T*C*G*T*T*T*T*G*T*C*G*T*T*T*T*G*T*C*G*T*T-3′/HEG/HEG/TEG Cholesteryl Ester/ (SEQ ID NO: 1), wherein the C is 2′-deoxy-P-cytidylyl, G is 2′-deoxy-P-guanylyl, T is 2′-deoxy-P-thymidylyl, HEG is Hexa(ethylene glycol)phosphodiester, TEG Cholesteryl Ester is (N-cholesteryl-3-aminopropyl)-triethyleneglycol-glyceryl-1-O-phosphodiester and * represents a phosphorothioate internucleotide linkage.

CpG-7909-SNA is a medicinal product for the treatment of advanced solid tumors. CpG-SNA exhibits a suite of mechanistic and functional properties that make it ideal for agonizing TLR9. First, SNAs are more efficiently taken into and concentrated in endosomes compared to linear oligonucleotides (i.e., oligonucleotides not in the SNA configuration). Second, the oligonucleotides delivered as a part of SNAs cause an enhanced cytokine response, both in magnitude and duration, compared to linear oligonucleotides. Third, the SNA projects its oligonucleotides outward, allowing it to act upon TLRs directly. This arrangement of nucleic acids in the SNA contrasts with other oligonucleotide delivery systems where the oligonucleotides are held internally, for example, inside virus-like particles.

In preclinical mouse models of anti-PD-1-resistant tumors, such as the breast EMT-6 model, anti-PD-1 antibodies showed no activity, whereas the combination of CpG-SNA and anti-PD-1 antibodies demonstrated significant tumor growth inhibition and prolonged mouse survival. Further, CpG-SNA outperformed linear oligonucleotides at the same dose.

Four dose levels of CpG-7909-SNA have been explored in a phase 1a first-in-human healthy volunteer study. Single doses were administered at 5-18.8 μg/kg subcutaneously (SC). There were no dose limiting toxicities (DLTs) and no serious adverse events (SAEs) in the study. CpG-SNA was undetectable in serum at all dose levels tested.

### Dosage and Administration

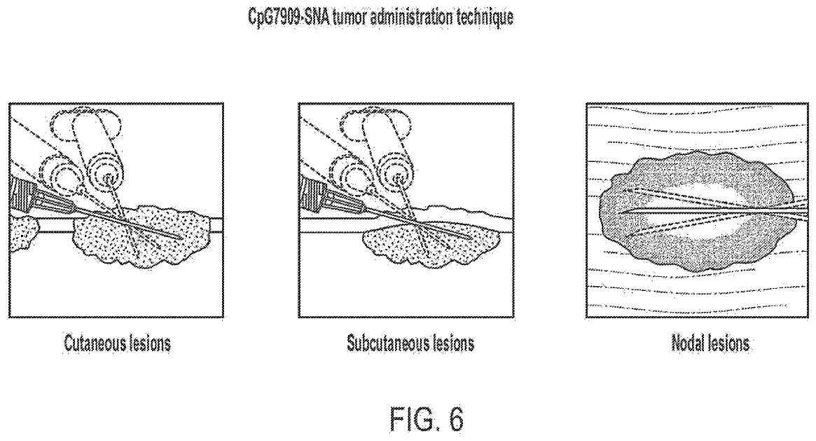

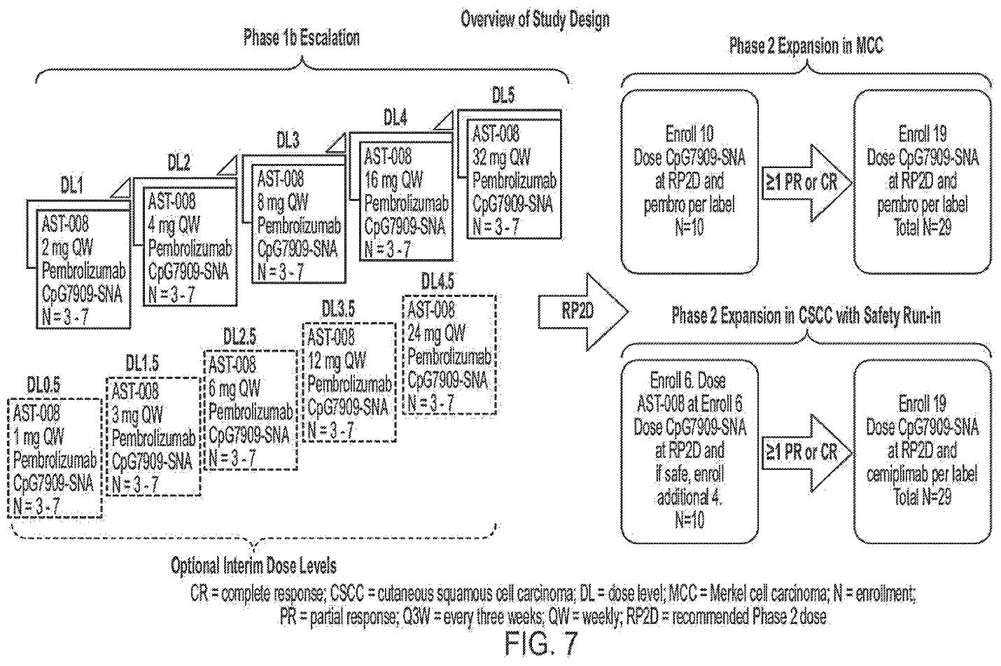

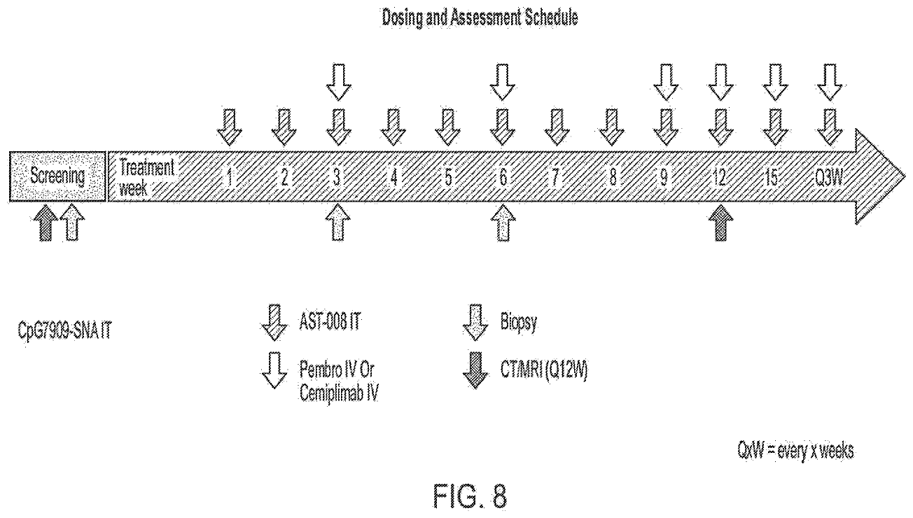

CpG-7909-SNA can be administered intratumorally (IT) into accessible basal cell carcinoma lesions, which are amenable to repeat administrations in cohorts of escalating doses of 2, 4, 8, 16, and 32 mg starting on day 1 of cycle 1. CpG-7909-SNA can be administered IT into cutaneous lesions, subcutaneous lesions, and/or nodal lesions, as shown in FIG. 6. One witness lesion would remain un-injected to allow for the observation of abscopal effects. Intermediate/de-escalation dose levels such as 1, 3, 6, 12, and 24 mg may also be explored.

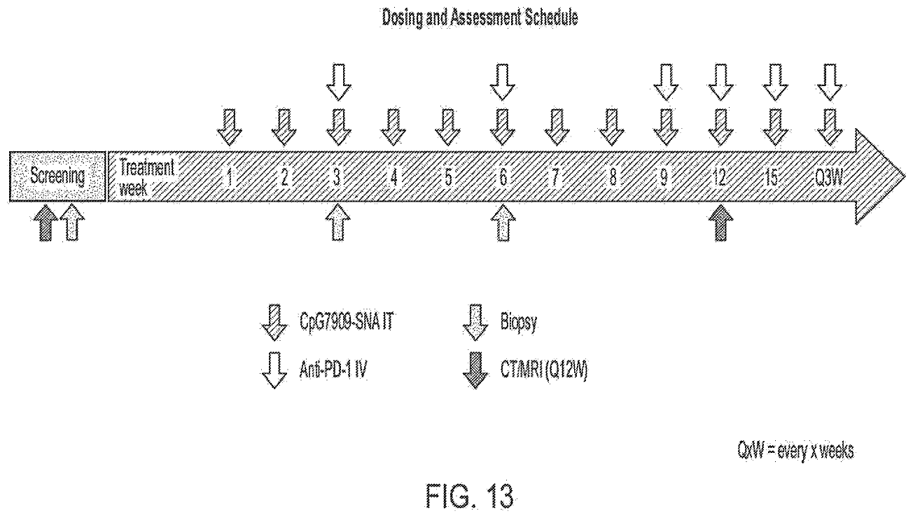

Cycle 1 (CpG-7909-SNA monotherapy) can be 2 weeks long and comprise IT injections on day 1 and 8. All other cycles can be 3 weeks long. For cycles 2 and 3, CpG-7909-SNA can be administered on a weekly basis, on days 1, 8, and 15 of each 3-week cycle. Starting at cycle 4, CpG-7909-SNA can be administered every three weeks starting on day 1 until lack of clinical benefit or disease progression. A corresponding representative dosing and assessment schedule is shown in FIG. 13.

Cemiplimab can be administered at a dose of 350 mg as an intravenous (IV) infusion over 30 minutes, on the same working day as the CpG-7909-SNA injection, every 3 weeks starting at cycle 2 day 1. Alternatively, another checkpoint inhibitor, such as another anti-PD-1 antibody or an anti-PD-L1 antibody, as disclosed herein, can be administered at a therapeutically effective dose as an IV infusion over 30 minutes, on the same working day as the CpG7909-SNA injection. Non-limiting examples of other checkpoint inhibitors that can be administered include pembrolizumab and avelumab.

### Study Design

A clinical study can be a classical 3+3 design, ascending dose, phase 1b study of CpG-7909-SNA combined with pembrolizumab or cemiplimab in BCC patients. Patients can be dosed twice with CpG-7909-SNA as a monotherapy before adding pembrolizumab or cemiplimab, which can be added starting at the second cycle.

### Example 7

Patients with locally advanced or metastatic basal cell carcinoma (BCC) can be treated with CpG-7909-SNA alone or in combination with anti-PD-1 antibody (e.g., cemiplimab). First line hedgehog pathway inhibitors are typically not well-tolerated in such patients, and BCC tumors typically exhibit low response durability after such treatments.

Patients with BCC who can be treated with CpG-7909-SNA alone or in combination with anti-PD-1 antibody (e.g., cemiplimab) may fall into one or more of the following categories: patients who are not responsive to or show progressive disease following treatment with imiquimod; patients who are not responsive to or show progressive disease following treatment with anti-PD-1 antibody (e.g., cemiplimab); and patients who are not responsive to or show progressive disease following treatment with TLR9 agonist (e.g., linear CpG-7909 oligonucleotide). These patient populations represent non-limiting examples of patients with BCC that may benefit from treatment with CpG-7909-SNA and a checkpoint inhibitor, such as cemiplimab or pembrolizumab.

Treatment of patients with BCC (e.g., locally advanced BCC, metastatic BCC, and/or superficial BCC lesions) using CpG-7909-SNA alone or in combination with anti-PD-1 antibody (e.g., cemiplimab, pembrolizumab) may result in suppression of tumor growth, regression of a tumor lesion or tumor lesions, or complete elimination of one or more tumor lesions in patient with BCC (e.g., locally advanced BCC, metastatic BCC, and/or superficial BCC lesions).

### REFERENCES

Aryan Z, Holgate S T, Radzioch D, et al. A new era of targeting the ancient gatekeepers of the immune system: toll-like agonists in the treatment of allergic rhinitis and asthma. International Archives of Allergy and Immunology. 2014; 164, 46-63.
Cohen E, Milhem M, Ribas A, et al. Phase 1b/2, Open-Label, Multicenter Study of Intratumoral SD-101 in Combination with Pembrolizumab in Anti-PD-1 Treatment-Naïve Patients with Recurrent or Metastatic Head and Neck Squamous Cell Carcinoma. Presented at: 2018 American Association for Cancer Research (AACR) Annual Meeting; Apr. 14-18, 2018; Chicago, IL. Abstract CT098.
Diab A, Haymaker C, Uemura M, et al. A Phase 1/2 trial of intratumoral (i.t.) IMO-2125 (IMO) in combination with checkpoint inhibitors (CPI) in PD-(L)1-refractory melanoma. Ann Oncol 2017; 28(suppl_5):v403-v427.
Eisenhauer E A, Therasse P, Bogaerts J, et al. 2009. New response evaluation criteria in solid tumours: Revised RECIST guideline (version 1.1). Eur J Cancer. 45(2):228-247.
Keytruda® United States Prescribing Information 2018
Krieg A M. CpG still rocks! Update on an accidental drug. Nucleic Acid Ther. 2012; 22, 77-89.
Libtayo® United States Prescribing Information 2019.
Lee D W, Gardner R, Porter D L, et al. Current concepts in the diagnosis and management of cytokine release syndrome. Blood 2014; 124(2):188-195.
Lentz H J. 2007. Management and preparedness for infusion and hypersensitivity reactions. Oncologist. 12:601-609.
Levey A S, Stevens L A, Schmid C H, et al. A new equation to estimate glomerular filtration rate. Ann Intern Med. 2009; 150(9):604-612.
Milhem M, Gonzales R, Medina T, et al. Intratumoral toll-like receptor 9 (TLR9) agonist, CMP-001, in combination with pembrolizumab can reverse resistance to PD-1 inhibition in a phase Ib trial in subjects with advanced melanoma. Presented at: AACR Annual Meeting 2018; Apr. 14-18, 2018; Chicago, Illinois. Abstract CT0101.
Nallagatla S R, Anderson B R, Kang R, et al. Abstract 3758: TLR9 agonist SNA-induced innate and adaptive immune responses in tumor microenvironment enhance checkpoint inhibitor antitumor activity in mouse tumor models. AACR Annual Meeting 2018; Apr. 14-18, 2018; Chicago, IL.
Opdivo® United States Prescribing Information 2018
Poh A. Warming “cold” melanoma with TLR9 agonists. Cancer Discov. 2018a June; 8(6):670. doi: 10.1158/2159-8290.CD-ND2018-004. Epub 2018 Apr. 18.
Ribas A, Medina T, Kummar S, et al. Durability of Responses to SD-101 in Combination with Pembrolizumab in Advanced Metastatic Melanoma: Results of a Phase 1b, Multicenter Study. Presented at: 2018 American Association for Cancer Research (AACR) Annual Meeting; Apr. 14-18, 2018b; Chicago, IL. Abstract CT139.
Sampson H A, Muñoz-Furlong A, Campbell R L, et al. Second symposium on the definition and management of anaphylaxis: summary report—Second National Institute of Allergy and Infectious Disease/Food Allergy and Anaphylaxis Network symposium. J Allergy Clin Immunol. 2006; 117(2):391-7.
Sato-Kaneko F, Yao S, Ahmadi A, et al. Combination immunotherapy with TLR agonists and checkpoint inhibitors suppresses head and neck cancer. JCI Insight. 2017:2(18):e93397. https://doi.org/10.1172/jci.insight.93397.
Schmidt M, Kapp K, Volz B et al. Combination of TLR9 agonist lefitolimod/MGN1703 with checkpoint inhibitors for cancer immunotherapy. J Clin Oncol 2017; 35 (4_suppl):a634.
Simon R, Designs for efficient clinical trials. Oncology (Williston Park) 1989; 3(7):43-9; discussion 51-3.
Thompson J A, Kuzel T, Drucker B J, et al. Safety and efficacy of PF-3512676 for the treatment of stage IV renal cell carcinoma: an open-label, multicenter phase I/II study. Clinical Genitourinary Cancer. 2009; 7, E58-65.
Wang S, Campos J, Naik E, et al. Intratumoral Treatment With A Highly Interferogenic TLR9 Agonist Reverts Tumor Escape From PD-1 Blockade. [Abstract]. In: Proceedings Of The 107th Annual Meeting Of The American Association For Cancer Research; 2016 Apr. 16-20; New Orleans, LA. Philadelphia (PA): AACR; Cancer Res 2016; 76(14 Suppl):Abstract Nr 2322.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A method for treating a disease comprising:
administration of an agonist of a tumor necrosis factor receptor superfamily (TNFRSF) member and a spherical nucleic acid (SNA) to a subject to treat a disease in the subject,
wherein the SNA comprises a core and an oligonucleotide shell comprised of CpG oligonucleotides positioned on the exterior of the core, and
wherein the administration of the SNA comprises administration once a week, once every three weeks, or once a week followed by once every three weeks.

2. The method of claim 1, wherein at least one CpG oligonucleotide is linked to the exterior surface of the core through a spacer or wherein each CpG oligonucleotide is linked to the exterior surface of the core through a spacer, and wherein the core has a diameter of less than 40 nm.

3. The method of claim 1 or claim 2, wherein the core is a liposome core.

4. The method of any one of claims 1-3, wherein the disease is cancer.

5. The method of any one of claims 1-4, wherein administration of the SNA comprises administration once a week for one week to 12 weeks, followed by once every three weeks.

6. The method of any one of claims 1-5, wherein administration of the SNA comprises administration once a week for nine weeks, followed by once every three weeks.

7. The method of any one of claims 1-6, wherein administration of the agonist of a TNFRSF member comprises administration once a week, once every two weeks, once every three weeks, once every four weeks, or a combination thereof.

8. The method of claim 7, wherein administration of the agonist of a TNFRSF member comprises administration once every three weeks.

9. The method of claim 7, wherein administration of the agonist of a TNFRSF member comprises administration once a week.

10. The method of any one of claims 1-9, wherein administration of the SNA is via at least one route of administration chosen from intratumoral administration to a solid tumor or tumor lesion, subcutaneous administration, internal implantation and intravenous administration.

11. The method of any one of claims 1-10, wherein administration of the agonist of a TNFRSF member is via at least one route of administration chosen from intratumoral administration to a solid tumor or tumor lesion, subcutaneous administration and intravenous administration.

12. The method of any one of claims 1-11, wherein administration of the SNA is via at least one route of administration chosen from intratumoral administration to a solid tumor or tumor lesion, subcutaneous administration, internal implantation and intravenous administration, and administration of the agonist of a TNFRSF member is via intravenous administration.

13. The method of any one of claims 1-11, wherein administration of the SNA is via at least one route of administration chosen from intratumoral administration to a solid tumor or tumor lesion, subcutaneous administration, internal implantation and intravenous administration, and administration of the agonist of a TNFRSF member is via at least one route of administration chosen from intratumoral administration to a solid tumor or tumor lesion and subcutaneous administration.

14. The method of any one of claims 1-13, further comprising administering an immune checkpoint inhibitor to the subject.

15. The method of any one of claims 1-14, wherein administration of the SNA is within 24 hours of administration of the agonist of a TNFRSF member.

16. The method of claim 14 or claim 15, wherein administration of the SNA is within 24 hours of administration of the immune checkpoint inhibitor.

17. The method of any one of claims 14-16, wherein administration of the SNA is within 24 hours of administration of the immune checkpoint inhibitor and within 24 hours of administration of the agonist of a TNFRSF member.

18. The method of any one of claims 4-17, wherein the cancer in the subject is not responsive to treatment with an immune checkpoint inhibitor alone or wherein the cancer in the subject is resistant to treatment with an immune checkpoint inhibitor alone.

19. The method of any one of claims 1-18, wherein administration of the SNA is at a dose of or about between 1 mg and 50 mg.

20. The method of any one of claims 1-19, wherein administration of the SNA is at a dose of or about between 2 mg and 32 mg.

21. The method of any one of claims 14-20, wherein administration of the immune checkpoint inhibitor is at a dose of or about between 150 mg and 850 mg.

22. The method of any one of claims 1-21, wherein administration of the agonist of a TNFRSF member is at a dose of or about between 7 mg and 1400 mg.

23. The method of any one of claims 4-22, wherein the cancer is skin cancer, biliary tract cancer, brain cancer, breast cancer, cervical cancer, choriocarcinoma, colon cancer, endometrial cancer, esophageal cancer, gastric cancer, an intraepithelial neoplasm, leukemia, lymphoma, liver cancer, lung cancer, oral cancer, ovarian cancer, pancreatic cancer, prostate cancer, rectal cancer, testicular cancer, thyroid cancer, renal cancer, multiple myeloma, or bladder cancer.

24. The method of claim 23, wherein the cancer is cutaneous squamous cell carcinoma, Merkel cell carcinoma, basal cell carcinoma, melanoma, hepatocellular carcinoma, neuroblastoma, glioma, glioblastoma multiforme, pancreatic adenocarcinoma, hormone refractory prostate adenocarcinoma, anaplastic thyroid cancer, hairy cell leukemia, chronic myelogenous leukemia, cutaneous T-cell leukemia, renal cell carcinoma, clear cell renal cell carcinoma, or non-small cell lung cancer (NSCLC).

25. The method of any one of claims 4-22, wherein the cancer is cutaneous squamous cell carcinoma, Merkel cell carcinoma, melanoma, or basal cell carcinoma (BCC).

26. The method of any one of claims 4-22, wherein the cancer is liver cancer, optionally wherein the liver cancer is hepatocellular carcinoma (HCC).

27. The method of any one of claims 4-22, wherein the cancer is not breast cancer.

28. The method of any one of claims 4-22, wherein the cancer is a sarcoma.

29. The method of claim 28, wherein the sarcoma is pleomorphic sarcoma, gastrointestinal stromal tumor (GIST), liposarcoma, leiomyosarcoma, synovial sarcoma, malignant peripheral nerve sheath tumor, rhabdomyosarcoma, angiosarcoma, fibrosarcoma, dermatofibrosarcoma protuberans, epithelioid sarcoma, myxoma, mesenchymoma, vascular sarcoma, neurilemmoma, bone sarcoma, osteosarcoma, Ewing's sarcoma, chondrosarcoma, Kaposi sarcoma, solitary fibrous tumor, chordoma, desmoid-type fibromatosis, fibroblastic sarcoma, giant cell tumor of the bone, gynecological sarcoma, soft tissue sarcoma, angioleiomyoma, leiomyoma, smooth muscle sarcoma, or fibrohistiocytic sarcoma.

30. The method of any one of claims 1-29, wherein the subject has a solid tumor or a tumor lesion that can be injected intratumorally via one or more of palpation and ultrasound.

31. The method of claim 30, wherein the solid tumor or tumor lesion is on or near the skin, on or near cutaneous soft tissue, on or near subcutaneous soft tissue, and/or in or near a lymph node.

32. The method of any one of claims 1-31, wherein administration of the SNA is to at least one lesion chosen from a cutaneous tumor lesion, a subcutaneous tumor lesion and a nodal lesion.

33. The method of any one of claims 2-32, wherein the spacer is or comprises an oligoethylene glycol.

34. The method of claim 33, wherein the oligoethylene glycol is a hexaethylene glycol.

35. The method of any one of claims 1-34, wherein the SNA includes between about 25 and about 35 CpG oligonucleotides.

36. The method of any one of claims 1-35, wherein at least one CpG oligonucleotide comprises or consists of the nucleic acid sequence of CpG-7909 or wherein each CpG oligonucleotide comprises or consists of the nucleic acid sequence of CpG-7909.

37. The method of any one of claims 1-36, wherein at least one CpG oligonucleotide comprises or consists of the nucleic acid sequence 5′-TCGTCGTTTTGTCGTTTTGTCGTT-3′ (SEQ ID NO: 1) or wherein each CpG oligonucleotide comprises or consists of the nucleic acid sequence 5′-TCGTCGTTTTGTCGTTTTGTCGTT-3′ (SEQ ID NO: 1).

38. The method of any one of claims 1-37, wherein at least one CpG oligonucleotide comprises or consists of the sequence 5′-T*C*G*T*C*G*T*T*T*T*G*T*C*G*T*T*T*T*G*T*C*G*T*T-3′/HEG/HEG/TEG Cholesteryl Ester/ (SEQ ID NO: 1) or wherein each CpG oligonucleotide comprises or consists of the sequence 5′-T*C*G*T*C*G*T*T*T*T*G*T*C*G*T*T*T*T*G*T*C*G*T*T-3′/HEG/HEG/TEG Cholesteryl Ester/ (SEQ ID NO: 1).

39. The method of any one of claims 14-38, wherein the immune checkpoint inhibitor is an anti-PD-1 antibody, an anti-PD-L1 antibody, or an anti-CTLA4 antibody.

40. The method of claim 39, wherein the immune checkpoint inhibitor is an anti-PD-1 antibody.

41. The method of claim 40, wherein the anti-PD-1 antibody is retifanlimab.

42. The method of any one of claims 1-41, wherein the TNFRSF member is OX40.

43. The method of any one of claims 1-42, wherein the agonist of TNFRSF member is an agonist of OX40.

44. The method of claim 43, wherein the agonist of OX40 is an anti-OX40 antibody.

45. The method of claim 44, wherein the anti-OX40 antibody is INCAGN1949.

46. The method of any one of claims 1-45, wherein administration of the SNA is within 12 hours of administration of the agonist of a TNFRSF member.

47. The method of any one of claims 14-46, wherein administration of the SNA is within 12 hours of administration of the immune checkpoint inhibitor.

48. The method of any one of claims 14-47, wherein administration of the SNA is within 12 hours of administration of the immune checkpoint inhibitor and within 12 hours of administration of the agonist of a TNFRSF member.

49. The method of any one of claims 1-48, wherein the core has a diameter that is less than about 30 nm.

50. The method of any one of claims 1-48, wherein the core has a diameter that is between about 15 nm and about 40 nm.

51. The method of any one of claims 1-48, wherein the core has a diameter that is between about 15 nm and about 30 nm.

52. The method of any one of claims 14-51, wherein administration of the SNA and the immune checkpoint inhibitor is substantially at the same time.

53. The method of any one of claims 1-51, wherein administration of the SNA is prior to administration of the agonist of a TNFRSF member.

54. The method of any one of claims 14-51, wherein administration of the SNA is prior to administration of the immune checkpoint inhibitor or prior to administration of the immune checkpoint inhibitor and after administration of the agonist of a TNFRSF member.

55. The method of any one of claims 1-51, wherein administration of the SNA is after administration of the agonist of a TNFRSF member.

56. The method of any one of claims 14-51, wherein administration of the SNA is after administration of the immune checkpoint inhibitor or after administration of the immune checkpoint inhibitor and after administration of the agonist of a TNFRSF member.

57. The method of any one of claims 1-56, wherein administration of the SNA and the agonist of a TNFRSF member, or administration of the SNA, the agonist of a TNFRSF member and the immune checkpoint inhibitor results in one or more of increased cytokine expression, increased chemokine expression, or increased immune cell activation by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99%, at least or about 100%, at least or about 150%, at least or about 2-fold, at least or about 3-fold, at least or about 4-fold, at least or about 5-fold, at least or about 6-fold, at least or about 7-fold, at least or about 8-fold, at least or about 9-fold, at least or about 10-fold, at least or about 15-fold, at least or about 20-fold, at least or about 30-fold, at least or about 40-fold, at least or about 50-fold or more, relative to a reference level.

58. The method of any one of claims 1-57, wherein administration of the SNA and the agonist of a TNFRSF member, or administration of the SNA, the agonist of a TNFRSF member and the immune checkpoint inhibitor increases an immune cell population in a solid tumor or tumor lesion in the subject.

59. The method of any one of claims 4-58, wherein the cancer in the subject is progressive disease and administration of the SNA and the agonist of a TNFRSF member, or the administration of the SNA, the immune checkpoint inhibitor and the agonist of a TNFRSF member renders the cancer stable disease.

60. The method of any one of claims 1-59, wherein each CpG oligonucleotide comprises at least one phosphorothioate internucleotide linkage.

61. The method of any one of claims 1-60, wherein each internucleotide linkage of each CpG oligonucleotide is a phosphorothioate internucleotide linkage.

62. The method of any one of claims 1-61, wherein the CpG oligonucleotides do not comprise a nucleotide in L-conformation.

63. The method of any one of claims 1-62, wherein the CpG oligonucleotides consist of nucleotides in D-conformation.

64. A method for treating cancer comprising:
administering to a subject a spherical nucleic acid (SNA) and a checkpoint inhibitor to treat a cancer in the subject,
wherein the SNA comprises a core and an oligonucleotide shell comprised of CpG oligonucleotides positioned on the exterior of the core, wherein the SNA is administered to the subject at a dose of at least about 2 mg to one solid tumor or tumor lesion or divided among two or more solid tumors or tumor lesions,
wherein the SNA is administered within 24 hours of administration of the checkpoint inhibitor.

65. A method for treating cancer comprising:
administering to a subject a spherical nucleic acid (SNA) and a checkpoint inhibitor to treat cancer in the subject,
wherein the SNA comprises a core and an oligonucleotide shell comprised of CpG oligonucleotides positioned on the exterior of the core, wherein the SNA is administered at a dose of between 2 mg and 32 mg once a week or every three weeks,
wherein the checkpoint inhibitor is administered at a dose of between 180 and 370 mg every three weeks, or at a dose of between 700 mg and 900 mg every two weeks,
wherein the SNA is administered within 24 hours of administration of the checkpoint inhibitor, and
wherein the SNA and the checkpoint inhibitor are administered through different routes of administration, wherein the cancer is basal cell carcinoma (BCC).

66. A method for treating cancer comprising:
administering a therapeutic dose of a checkpoint inhibitor and a therapeutic dose of a spherical nucleic acid (SNA) to treat cancer in the subject, wherein the SNA comprises a CpG oligonucleotide linked through a spacer to an exterior surface of a liposome core having a diameter of less than 40 nm, wherein the SNA is administered by intratumoral (IT) administration into one solid tumor or tumor lesion or divided among two or more solid tumors or tumor lesions at a dose of between 2 mg and 32 mg and wherein the checkpoint inhibitor is administered by intravenous (IV) administration at a dose of between 180 and 800 mg.

67. The method of claim 64 or claim 66, wherein the cancer is BCC.

68. The method of claim 64 or claim 66, wherein the cancer is melanoma.

69. The method of claim 64 or claim 66, wherein the cancer is liver cancer.

70. The method of claim 69, wherein the liver cancer is hepatocellular carcinoma (HCC).

71. The method of claim 64 or claim 65, wherein the SNA is administered by subcutaneous administration or intratumoral administration to a solid tumor and wherein the checkpoint inhibitor is administered by IV infusion.

72. The method of any one of claims 64-71, wherein the cancer in the subject is not responsive to treatment with the checkpoint inhibitor alone or wherein the cancer in the subject is resistant to treatment with the checkpoint inhibitor alone.

73. The method of any one of claims 64-72, wherein the subject has not received a small molecule or tyrosine kinase inhibitor within 2 weeks or 5 half-lives (whichever is longer) prior to the first dose of the SNA, has not received chemotherapy within 3 weeks prior to the first dose of the SNA, has not received biological cancer therapy within 3 weeks prior to the first dose of the SNA, has not received nitrosourea or radioisotope within 6 weeks prior to first dose of the SNA, has not recovered from an adverse event (G1) or has not been identified as experiencing an adverse event due to cancer therapeutics administered more than 4 weeks prior to the first dose of the SNA.

74. The method of any one of claims 64-73, wherein the SNA is administered at a dose of or about 16 mg.

75. The method of any one of claims 64-73, wherein the SNA is administered at a dose of or about 32 mg.

76. The method of any one of claims 64-75, wherein the checkpoint inhibitor is administered at a dose between 100 mg and 1000 mg.

77. The method of any one of claims 1-59, wherein the checkpoint inhibitor is administered at a dose of or about 200 mg, 350 mg, or 800 mg.

78. The method of any one of claims 1-77, wherein the subject has a solid tumor or a tumor lesion that can be injected intratumorally via one or more of palpation or ultrasound.

79. The method of claim 78, wherein the solid tumor or tumor lesion is on or near the skin, on or near cutaneous soft tissue, on or near subcutaneous soft tissue, and/or in or near a lymph node.

80. The method of claim 78 or 79, wherein the SNA is administered to one or more of a cutaneous tumor lesion, a subcutaneous tumor lesion or a nodal lesion.

81. The method of any one of claims 1, 2 and 4-80, wherein the core is a liposome core.

82. The method of any one of claims 3-81, wherein the liposome core is about 15 nanometers (nm) to about 30 nm in diameter.

83. The method of any one of claims 1, 2, and 4-82, wherein each CpG oligonucleotide comprises a spacer.

84. The method of claim 83, wherein the spacer consists of or comprises an oligoethylene glycol.

85. The method of claim 84, wherein the oligoethylene glycol is a hexaethylene glycol.

86. The method of any one of claims 1-85, wherein the SNA has 25 to 35 CpG oligonucleotides positioned on the exterior surface of the core.

87. The method of any one of claims 1-86, wherein the CpG oligonucleotides comprise or consist of the nucleotide sequence 5′-TCGTCGTTTTGTCGTTTTGTCGTT-3′ (SEQ ID NO: 1).

88. The method of any one of claims 1-87, wherein the CpG oligonucleotides comprise or consist of the sequence 5′-T*C*G*T*C*G*T*T*T*T*G*T*C*G*T*T*T*T*G*T*C*G*T*T-3′/HEG/HEG/TEG Cholesteryl Ester/ (SEQ ID NO: 1).

89. The method of any one of claims 1-88, wherein the checkpoint inhibitor is a programmed death (PD) 1 antibody or a programmed death-ligand (PD-L) 1 antibody.

90. The method of any one of claims 1-89, wherein the checkpoint inhibitor is pembrolizumab, avelumab, or cemiplimab.

91. The method of any one of claims 3-90, wherein the SNA is administered within 24 hours of administration of the checkpoint inhibitor.

92. The method of any one of claims 1-91, wherein the SNA is administered within 12 hours of administration of the checkpoint inhibitor.

93. The method of any one of claims 3-92, wherein the liposome core is less than 30 nm in diameter.

94. The method of any one of claims 3-92, wherein the liposome core is about 15 nm to 40 nm in diameter.

95. The method of any one of claims 1-90 or 93, wherein the SNA and the checkpoint inhibitor are administered substantially at the same time.

96. The method of any one of claims 1-90, 93, or 94, wherein the SNA is administered prior to administration of the checkpoint inhibitor.

97. The method of any one of claims 1-90, 93, or 94, wherein the SNA is administered after the administration of the checkpoint inhibitor.

98. A method for treating cancer, comprising:
administering to a subject a spherical nucleic acid (SNA) and a checkpoint inhibitor to treat the cancer in the subject,
wherein the SNA comprises a core and an oligonucleotide shell comprised of CpG oligonucleotides positioned on the exterior of the core, wherein the SNA is administered to the subject at a dose of between about 16 mg to about 32 mg to one solid tumor or tumor lesion or divided among two or more solid tumors or tumor lesions, wherein the SNA is administered within 24 hours of administration of the checkpoint inhibitor, and wherein the cancer is basal cell carcinoma (BCC), melanoma, or liver cancer.

99. A method for treating cancer, comprising:
administering to a subject a spherical nucleic acid (SNA) and a checkpoint inhibitor to treat the cancer in the subject,
wherein the SNA comprises a core and an oligonucleotide shell comprised of CpG oligonucleotides positioned on the exterior of the core, wherein the SNA is administered at a dose of between about 16 mg to about 32 mg once a week or once every three weeks,
wherein the checkpoint inhibitor is administered at a dose of between 180 mg and 370 mg every three weeks or at a dose of between 700 mg and 900 mg every two weeks,
wherein the SNA is administered within 24 hours of the administration of the checkpoint inhibitor,
wherein the SNA and the checkpoint inhibitor are administered through different routes of administration, and wherein the cancer is basal cell carcinoma (BCC), melanoma or liver cancer.

100. A method for treating cancer, comprising:
administering a therapeutic dose of a spherical nucleic acid (SNA) comprising a CpG oligonucleotide linked through a spacer to an exterior surface of a liposome core having a diameter of less than about 40 nanometers (nm) and a checkpoint inhibitor, wherein the SNA is administered by intratumoral (IT) administration into two or more solid tumors or tumor lesions at a dose of between about 16 mg and about 32 mg and the checkpoint inhibitor is administered by intravenous (IV) administration at a dose of between 180 and 370 mg, between 700 mg and 900 mg, or between 800 mg and 1700 mg, and wherein the cancer is basal cell carcinoma (BCC), melanoma, or liver cancer.

101.112A. The method of any one of claims 98-100, wherein the cancer is BCC.

102.112B. The method of any one of claims 98-100, wherein the cancer is melanoma.

103.112C. The method of any one of claims 98-100, wherein the cancer is liver cancer, optionally wherein the liver cancer is hepatocellular carcinoma (HCC).

104.112D. The method of any one of claims 98-103, wherein the checkpoint inhibitor is a PD-1 inhibitor or a PD-L1 inhibitor.

105.112E. The method of claim 104, wherein the PD-L1 inhibitor is atezolizumab (Tecentriq).

106. The method of claim 105, wherein the PD-L1 inhibitor is administered at a dose of or about 1680 mg, 1200 mg, or 840 mg.

107. The method of claim 106, further comprising administration of a VEGF antibody.

108. The method of claim 107, wherein the VEGF antibody is bevacizumab (Avastin).

109. The method of claim 108, wherein the VEGF antibody is administered at a dose of or about 15 mg/kg.

110. The method of claim 104, wherein the PD-1 inhibitor is pembrolizumab (Keytruda).

111. The method of claim 104, wherein the PD-1 inhibitor is nivolumab (Opdivo).

112. The method of claim 111, wherein the PD-1 inhibitor is administered at a dose of 1 mg/kg, 240 mg, or 480 mg.

113. The method of claim 112, further comprising administration of a CTLA-4 inhibitor.

114. The method of claim 113, wherein the CTLA-4 inhibitor is ipilimumab (Yervoy).

115. The method of claim 114, wherein the CTLA-4 inhibitor is administered at a dose of 3 mg/kg or 10 mg/kg.

116. The method of any one of claims 104-115, wherein the checkpoint inhibitor is administered via intravenous infusion.

117. The method of any one of claims 104-116, wherein the checkpoint inhibitor is administered once every two weeks, once every three weeks, once every four weeks or once every six weeks.

118. The method of any one of 114-117, wherein ipilimumab is administered once every three weeks, four times or at least four times.

119. The method of any one of claims 98-118, wherein the SNA is administered at a dose of or about 16 mg.

120. The method of any one of claims 98-118, wherein the SNA is administered at a dose of or about 32 mg.

121. The method of any one of claims 98-120, wherein the SNA is administered at a dose of or about 16 mg and wherein the checkpoint inhibitor is administered at a dose of or about 200 mg.

122. The method of any one of claims 98-120, wherein the SNA is administered at a dose of or about 32 mg and wherein the checkpoint inhibitor is administered at a dose of or about 200 mg.

123. The method of any one of claims 98-120, wherein the SNA is administered at a dose of or about 16 mg and wherein the checkpoint inhibitor is administered at a dose of or about 350 mg.

124. The method of any one of claims 98-120, wherein the SNA is administered at a dose of or about 32 mg and wherein the checkpoint inhibitor is administered at a dose of or about 350 mg.

125. The method of any one of claims 98-120 wherein the SNA is administered at a dose of or about 16 mg and wherein the checkpoint inhibitor is administered at a dose of or about 800 mg.

126. The method of any one of claims 98-120, wherein the SNA is administered at a dose of or about 32 mg and wherein the checkpoint inhibitor is administered at a dose of or about 800 mg.

127. The method of any one of claims 98-126, wherein administration of the SNA or the SNA in combination with the checkpoint inhibitor results in one or more of increased cytokine expression, increased chemokine expression, or increased immune cell activation by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99%, at least or about 100%, at least or about 150%, at least or about 2-fold, at least or about 3-fold, at least or about 4-fold, at least or about 5-fold, at least or about 6-fold, at least or about 7-fold, at least or about 8-fold, at least or about 9-fold, at least or about 10-fold, at least or about 15-fold, at least or about 20-fold, at least or about 30-fold, at least or about 40-fold, at least or about 50-fold or more, relative to a reference level.

128. The method of any one of claims 1-127, wherein the cancer in the subject is progressive disease and administration of the SNA or administration of the SNA in combination with the checkpoint inhibitor for the treatment of the cancer in the subject renders the cancer stable disease (SD).

129. The method of any one of claims 1-128, wherein the cancer is SD for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks.

130. The method of any one of claims 1-128, wherein the cancer is SD for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months.

131. The method of any one of claims 1-128, wherein the cancer is SD for at least one year, at least two years, at least three years, at least four years, at least five years, at least six years, at least seven years, at least eight years, at least nine years, at least 10 years, at least 11 years or at least 12 years.

132. The method of any one of claims 1-131, wherein the subject has at least one target lesion, at least two target lesions, at least three target lesions or at least four target lesions and wherein administration of the SNA or the SNA in combination with the checkpoint inhibitor decreases the diameter of at least one target lesion in the subject or decreases the sum of the diameters of two or more target lesions in the subject by at least or about 5%, at least or about 10%, at least or about 15%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 99% relative to a reference level.

133. The method of any one of claims 1-132, wherein the subject has at least one target lesion, at least two target lesions, at least three target lesions or at least four target lesions and wherein administration of the SNA or the SNA in combination with the checkpoint inhibitor results in partial response or results in complete response in at least one target lesion, at least two target lesions, at least three target lesions, or at least four target lesions in the subject, wherein the target lesion is a solid tumor or tumor lesion.

134. The method of any one of claims 1-133, wherein the treatment results in partial response or complete response in the subject for at least two weeks, at least four weeks, at least six weeks, at least eight weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, or at least 40 weeks.

135. The method of any one of claims 1-133, wherein the treatment results in partial response or complete response in the subject for at least two months, at least four months, at least six months, at least eight months, at least 10 months, at least 12 months, at least 14 months, at least 16 months, at least 18 months, at least 20 months, at least 22 months, at least 24 months, at least 26 months, at least 28 months, at least 30 months, at least 32 months, at least 34 months, at least 36 months, at least 38 months, or at least 40 months.

136. The method of any one of claims 1-133, wherein the treatment results in partial response or complete response in the subject for at least one year, at least two years, at least three years, at least four years, at least five years, at least six years, at least seven years, at least eight years, at least nine years, at least 10 years, at least 11 years or at least 12 years.


SEQUENCE LISTING (1 sequence)

SEQ ID NO: 1 - DNA, 24 bp, Artificial Sequence
  Synthetic
  misc_feature (1)..(24): may be modified by phosphorothioate internucleotide linkage
tcgtcgttttgtcgttttgtcgtt